SEQUENCE LISTING (419 sequences)

SEQ ID NO: 1 - protein, 22 aa, Artificial Sequence
  Polypeptide for minigene HBV.1 with epitope identity core 18
TLKAAAFLPSDFFPSVFLLSLG

SEQ ID NO: 2 - protein, 22 aa, Artificial Sequence
  Polypeptide for minigene pMIn1 with epitope identity core 18
TLKAAAFLPSDFFPSVKLTPLC

SEQ ID NO: 3 - protein, 21 aa, Artificial Sequence
  Polypeptide for minigene HCV1 with epitope identity core 132
ILGGWVDLMGYIPLVYLVAYQ

SEQ ID NO: 4 - protein, 20 aa, Artificial Sequence
  Polypeptide for minigene HCV2 with epitope identity core 132
VPGSRGDLMGYIPLVAKFVA

SEQ ID NO: 5 - protein, 9 aa, Artificial Sequence
  Oligopeptide
VLAEAMSQV

SEQ ID NO: 6 - protein, 9 aa, Artificial Sequence
  Oligopeptide
ILKEPVHGV

SEQ ID NO: 7 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXFXXL

SEQ ID NO: 8 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXFXXI

SEQ ID NO: 9 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXFXXM

SEQ ID NO: 10 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXFXXV

SEQ ID NO: 11 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXYXXL

SEQ ID NO: 12 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXYXXI

SEQ ID NO: 13 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXYXXM

SEQ ID NO: 14 - protein, 8 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXYXXV

SEQ ID NO: 15 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXFXXXL

SEQ ID NO: 16 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXFXXXI

SEQ ID NO: 17 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXFXXXM

SEQ ID NO: 18 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXFXXXV

SEQ ID NO: 19 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXYXXXL

SEQ ID NO: 20 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXYXXXI

SEQ ID NO: 21 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXYXXXM

SEQ ID NO: 22 - protein, 9 aa, Artificial Sequence
  Motif utilized for junctional minimization (murine Kb)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXYXXXV

SEQ ID NO: 23 - protein, 8 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXNXXL

SEQ ID NO: 24 - protein, 8 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXNXXI

SEQ ID NO: 25 - protein, 8 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXNXXM

SEQ ID NO: 26 - protein, 8 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
XXXXNXXV

SEQ ID NO: 27 - protein, 9 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXNXXXL

SEQ ID NO: 28 - protein, 9 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXNXXXI

SEQ ID NO: 29 - protein, 9 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXNXXXM

SEQ ID NO: 30 - protein, 9 aa, Artificial Sequence
  Motif utilized for junctional minimization (Db)
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
XXXXNXXXV

SEQ ID NO: 31 - protein, 9 aa, Artificial Sequence
  Motif utilized for junctional minimization (human A2)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXV

SEQ ID NO: 32 - protein, 9 aa, Artificial Sequence
  Motif utilized for junctional minimization (human A2)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXV

SEQ ID NO: 33 - protein, 10 aa, Artificial Sequence
  Motif utilized for junctional minimization (human A2)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXXV

SEQ ID NO: 34 - protein, 10 aa, Artificial sequence
  Motif utilized for junctional minimization (human A2)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXXV

SEQ ID NO: 35 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXK

SEQ ID NO: 36 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXR

SEQ ID NO: 37 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXY

SEQ ID NO: 38 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XIXXXXXXK

SEQ ID NO: 39 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XIXXXXXXR

SEQ ID NO: 40 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XIXXXXXXY

SEQ ID NO: 41 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXK

SEQ ID NO: 42 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXR

SEQ ID NO: 43 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXY

SEQ ID NO: 44 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XVXXXXXXK

SEQ ID NO: 45 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XVXXXXXXR

SEQ ID NO: 46 - protein, 9 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XVXXXXXXY

SEQ ID NO: 47 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XLXXXXXXXK

SEQ ID NO: 48 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XIXXXXXXXK

SEQ ID NO: 49 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XMXXXXXXXK

SEQ ID NO: 50 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XVXXXXXXXK

SEQ ID NO: 51 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XLXXXXXXXR

SEQ ID NO: 52 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XIXXXXXXXR

SEQ ID NO: 53 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XMXXXXXXXR

SEQ ID NO: 54 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XVXXXXXXXR

SEQ ID NO: 55 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XLXXXXXXXY

SEQ ID NO: 56 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XIXXXXXXXY

SEQ ID NO: 57 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XMXXXXXXXY

SEQ ID NO: 58 - protein, 10 aa, Artificial Sequence
  Motif utilized for junctional minimization (human A3/A11)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XVXXXXXXXY

SEQ ID NO: 59 - protein, 9 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XPXXXXXXL

SEQ ID NO: 60 - protein, 9 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XPXXXXXXI

SEQ ID NO: 61 - protein, 9 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XPXXXXXXM

SEQ ID NO: 62 - protein, 9 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XPXXXXXXV

SEQ ID NO: 63 - protein, 9 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XPXXXXXXF

SEQ ID NO: 64 - protein, 10 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XPXXXXXXXL

SEQ ID NO: 65 - protein, 10 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XPXXXXXXXI

SEQ ID NO: 66 - protein, 10 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XPXXXXXXXM

SEQ ID NO: 67 - protein, 10 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XPXXXXXXXV

SEQ ID NO: 68 - protein, 10 aa, Artificial Sequence
  Motif utilized for junctional minimization (human B7)
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XPXXXXXXXF

SEQ ID NO: 69 - protein, 13 aa, Artificial Sequence
  PADRE sequence
AKFVAAWTLKAAA

SEQ ID NO: 70 - protein, 9 aa, Artificial Sequence
  Oligopeptide
VLAEAMSQV

SEQ ID NO: 71 - protein, 9 aa, Artificial Sequence
  Oligopeptide
ILKEPVHGV

SEQ ID NO: 72 - protein, 9 aa, Artificial Sequence
  Oligopeptide
TLNFPISPI

SEQ ID NO: 73 - protein, 10 aa, Artificial Sequence
  Oligopeptide
SLLNATDIAV

SEQ ID NO: 74 - protein, 10 aa, Artificial Sequence
  Oligopeptide
QMAVFIHNFK

SEQ ID NO: 75 - protein, 11 aa, Artificial Sequence
  Oligopeptide
VTVYYGVPVWK

SEQ ID NO: 76 - protein, 8 aa, Artificial Sequence
  Oligopeptide
FPVRPQPL

SEQ ID NO: 77 - protein, 10 aa, Artificial Sequence
  Oligopeptide
YPLASLRSLF

SEQ ID NO: 78 - protein, 10 aa, Artificial Sequence
  Oligopeptide
VIYQYMDDLY

SEQ ID NO: 79 - protein, 9 aa, Artificial Sequence
  Oligopeptide
IYQEPFKNL

SEQ ID NO: 80 - protein, 9 aa, Artificial Sequence
  Oligopeptide
IWGCSGKLI

SEQ ID NO: 81 - protein, 277 aa, Artificial Sequence
  HIV-1090
MGMQVQIQSLFLLLLWVPGSRGKLVGKLNWAGAAILKEPVHGVNAACPKVSFEPIKIPIHYCAPAKAKFVAAWTLKAAAKAFPVRPQVPLGAAKLTPLCVTLGAAAVLAEAMSQVKVYLAWVPAHKGAAAAIFQSSMTKKTTLFCASDAKNIPYNPQSQGVVKHPVHAGPIANVTVYYGVPVWKKAAAQMAVFIHNFKNAAAYPLASLRSLFNLTFGWCFKLNRILQQLLFINAKIQNFRVYYRKAAVTIKIGGQLKKVPLQLPPLKAMTNNPPIPV

SEQ ID NO: 82 - DNA, 834 bp, Artificial Sequence
  Encoding sequence for HIV-1090
atgggaatgcaggtgcagatccagagcctgtttctgctcctcctgtgggtgcccggatccagaggaaagctggtgggcaaactcaactgggccggagctgcaatcctgaaggagcccgtccacggggtgaatgccgcttgccctaaagtcagcttcgaaccaattaagatccccattcattactgtgcacctgccaaagctaagtttgtggccgcttggaccctcaaggccgctgcaaaagccttcccagtgaggccccaggtgcctctgggcgccgctaaactcacaccactgtgcgtcactctgggagccgctgcagtgctggcagaggccatgtcccaagtgaaggtgtatctggcttgggtgcccgcccacaagggggccgctgcagccatctttcagtctagcatgaccaagaaaacaactctgttctgtgcctccgacgctaagaacatcccttataatccacagtctcagggcgtggtcaagcatcccgtgcacgccggacctattgctaacgtgaccgtgtactatggggtcccagtgtggaagaaagccgctgcacagatggccgtgtttattcacaatttcaaaaacgccgctgcataccccctcgccagcctgagatccctcttcaacctgacattcggctggtgctttaagctgaaccggatcctgcagcaactgctctttatcaatgctaaaatccagaacttccgcgtctactataggaaggctgcagtgactatcaaaattggcggacaactgaagaaagtgcctctccagctgccccctctcaaggcaatgaccaacaatccccctatcccagtctga

SEQ ID NO: 83 - protein, 280 aa, Artificial Sequence
  HIV-CPT
MGMQVQIQSLFLLLLWVPGSRGIPIHYCAPAKAAKIQNFRVYYRKAAVTIKIGGQLKKAKFVAAWTLKAAAKVPLQLPPLKAIFQSSMTKKLTPLCVTLGAQMAVFIHNFKGAKVYLAWVPAHKNAIPYNPQSQGVVKAILKEPVHGVGAAALTFGWCFKLNAVLAEAMSQVNRILQQLLFINAAACPKVSFEPIKVTVYYGVPVWKKAAHPVHAGPIANAAAYPLASLRSLFNAAATTLFCASDAKNKLVGKLNWANAAAFPVRPQVPLNMTNNPPIPV

SEQ ID NO: 84 - DNA, 843 bp, Artificial Sequence
  Encoding sequence for HIV-CPT
atggggatgcaggtgcagatccagagcctgtttctgctcctcctgtgggtgcccggatccagaggaatccccattcactactgcgcccctgctaaggcagccaaaatccagaacttcagggtgtattacagaaaggctgcagtcaccattaaaatcggcggacaactgaagaaagccaagtttgtggccgcttggacactcaaggccgctgcaaaggtcccactgcagctcccccctctgaaggccatcttccagagctccatgactaagaaactgaccccactgtgtgtgacactcggggcccagatggctgtgttcatccataattttaaaggcgccaaggtctacctggcttgggtgcccgcacacaagaacgccattccttacaatccacagtctcaaggagtggtcaaagctattctgaaggagcccgtgcacggggtgggcgccgctgcactcactttcggatggtgctttaaactgaacgccgtgctggctgaagccatgagccaggtcaatcggatcctgcagcaactgctcttcattaacgccgctgcatgtcctaaggtgtccttcgagccaatcaaagtgaccgtgtattacggggtccccgtgtggaagaaagccgctcatcctgtccacgcaggcccaatcgccaacgccgctgcatatcccctcgcctctctgcgcagcctgtttaacgccgctgcaacaaccctcttttgcgcctccgacgctaagaataaactggtgggaaagctgaactgggccaacgcagctgcattccctgtgaggccacaggtccccctcaatatgactaacaatccccctatcccagtgtga

SEQ ID NO: 85 - protein, 211 aa, Artificial Sequence
  HIV-FT
MQVQIQSLFLLLLWVPGSRGKLVGKLNWAMASDFNLPPVAIFQSSMTKVTIKIGGQLKRILQQLLFIMAVFIHNFKIPYNPQSQGVVTTLFCASDAKILKEPVHGVQMAVFIHNFKGAAVFIHNFKRCPKVSFEPIKIQNFRVYYRLTFGWCFKLQVPLRPMTYKMTNNPPIPVTVYYGVPVWKVLAEAMSQVIPIHYCAPAKLTPLCVTL

SEQ ID NO: 86 - DNA, 633 bp, Artificial Sequence
  Encoding sequence for HIV-FT
atgcaggtgcagatccagagcctgtttctgctcctcctgtgggtgcccggatccagaggaaagctggtggggaagctgaactgggccatggccagcgatttcaacctgccccccgtggccatcttccagagcagcatgaccaaggtgaccatcaagatcggggggcagctgaagaggatcctgcagcagctgctgttcatcatggccgtgttcatccacaacttcaagatcccctacaacccccagagccagggggtggtgaccaccctgttctgcgccagcgatgccaagatcctgaaggagcccgtgcacggggtgcagatggccgtgttcatccacaacttcaagggcgccgccgtgttcatccacaacttcaagaggtgccccaaggtgagcttcgagcccatcaagatccagaacttcagggtgtactacaggctgaccttcgggtggtgcttcaagctgcaggtgcccctgaggcccatgacctacaagatgaccaacaacccccccatccccgtgaccgtgtactacggggtgcccgtgtggaaggtgctggccgaggccatgagccaggtgatccccatccactactgcgcccccgccaagctgacccccctgtgcgtgaccctg

SEQ ID NO: 87 - protein, 585 aa, Artificial Sequence
  HIV-TC
MGMQVQIQSLFLLLLWVPGSRGYWQATWIPEWKAIFQSSMTKKVYLAWVPAHKNAACPKVSFEPIKHPVHAGPIANLTFGWCFKLNKMIGGIGGFIKFRDYVDRFYKAAARILQQLLFINTTLFCASDAKNQMVHQAISPRGAKLVGKLNWAGAAAIYETYGDTWKAAQVPLRPMTYKGAAAVTVLDVGDAYNAAARYLKDQQLLNTLNFPISPINMTNNPPIPVNAPYNTPVFAIKAAAVPLQLPPLKAAIPYNPQSQGVVKALLQLTVWGIGAAILKEPVHGVNAAAFPISPIETVKVWKEATTTLFKAAAVTIKIGGQLKKIYQEPFKNLKAAAVLAEAMSQVNLVGPTPVNIGAAAEVNIVTDSQYKAAAIPIHYCAPAKAVIYQYMDDLYKAAAQMAVFIHNFKNAATYQIYQEPFKPYNEWTLELKAKIQNFRVYYRKAFPVRPQVPLGAAAIWGCSGKLIKVMIVWQVDRNAAKAACWWAGIKAKFVAAWTLKAAAKLTPLCVTLNAAMASDFNLPPVKSLLNATDIAVNVTVYYGVPVWKKAAAAIIRILQQLKRAMASDFNLNAAAYPLASLRSLF

SEQ ID NO: 88 - DNA, 1758 bp, Artificial Sequence
  Encoding sequence for HIV-TC
atggggatgcaggtgcagatccagagcctgtttctgctcctcctgtgggtgcccggatctagaggatactggcaagctacttggattccagaatggaaagctatctttcaatcctcaatgacgaagaaggtatacctggcatgggtcccagcacacaagaacgccgcttgcccaaaggtgtcctttgaacccattaaacacccagtgcacgcagggccaatagcgaatttgacattcgggtggtgcttcaaactaaacaaaatgatcggcggcattggaggctttatcaagtttagagattacgtggaccgattctataaagccgctgcccgtatactccagcagctactattcatcaacaccactctcttctgcgcttcagacgctaagaaccaaatggtacaccaagccataagccctagaggagccaagctcgtagggaaattaaattgggcgggtgcagcagcaatctacgagacttacggcgatacctggaaagcagcccaggttccgttacgcccaatgacctataaaggcgcagcagcagtaacagttctagatgtaggagacgcttacaacgctgccgcaagatacctaaaagatcagcagttactcaacacactaaatttcccaattagcccgataaacatgacaaataacccaccaattcccgtcaatgctccctacaacactccagtattcgcaatcaaagccgctgctgtccccctgcagctccctcctctgaaagctgcgataccttacaacccacagagccaaggtgttgtcaaagcactgcttcagctaacagtttggggaattggtgctgcaattctaaaagagccagttcatggggttaacgccgccgccttcccaatcagtcctattgagactgtgaaagtatggaaagaagccacaaccacactttttaaggcagccgcagttacaattaaaatagggggccaacttaagaaaatataccaggaacctttcaagaatctcaaagccgctgcagtgctcgccgaggctatgtcacaggtgaatttggtcggaccaacacccgtaaacatcggagccgcagccgaagtgaacatagtcaccgactcacagtacaaagccgctgcaatacccatacattattgtgctcccgcaaaggccgtgatctatcaatatatggacgacctgtataaggccgccgcgcagatggcagtctttatccacaactttaaaaacgcagctacttatcagatctaccaggaaccattcaaaccgtacaatgagtggaccttggaactaaaggccaaaattcagaacttcagggtatattatagaaaagcatttccagtgaggccccaggtgcctctgggtgccgcagcaatatggggatgttctggaaaactgatcaaggtgatgattgtatggcaagtggacagaaatgcagctaaggcagcctgttggtgggcaggtataaaagcaaagttcgtggcagcatggacgcttaaagcagccgcaaaactcactcctctctgcgtgacacttaatgcagccatggcctctgatttcaaccttccccctgtaaaatccctgcttaatgcgacagatatcgcagtcaacgtaacagtatattatggcgtgccagtctggaaaaaagccgccgcggccataattcggatactgcagcagctgaaaagagctatggcgagtgacttcaacctgaatgcggccgcctaccccttggcatcgttaaggtcactattttga

SEQ ID NO: 89 - protein, 255 aa, Artificial Sequence
  HCV.1
MGMQVQIQSLFLLLLWVPGSRGLLFNILGGWVDLMGYIPLVYLVAYQATVILAGYGAGVRLIVFPDLGVHMWNFISGIYLLPRRGPRLYLVTRHADVVLVGGVLAALLFLLLADAFLLLADARVWMNRLIAFACTCGSSDLYLSAFSLHSYGVAGALVAFKLPGCSFSIFKTSERSQPRLIFCHSKKKFWAKHMWNFIPFYGKAIRMYVGGVEHRQLFTFSPRRRLGVRATRKVGIYLLPNRAKFVAAWTLKAAA

SEQ ID NO: 90 - DNA, 747 bp, Artificial Sequence
  Encoding sequence for HCV.1
gaattcgccgccaccatgcaggtgcagatccagagcctgtttctgctcctcctgtgggtgcccggatccagaggactgctgttcaacatcctgggggggtgggtggatctgatggggtacatccccctggtgtacctggtggcctaccaggccaccgtgatcctggccgggtacggggccggggtgaggctgatcgtgttccccgatctgggggtgcacatgtggaacttcatcagcgggatctacctgctgcccaggagaggacctagactgtacctggtgactagacacgctgatgtggtgctggtgggaggagtgctggctgctctgctgtttctgctgctggctgatgctttcctgctgctggctgatgctagagtgtggatgaacagactgatcgctttcgcttgtacatgtggaagctccgatctgtatctgagcgctttcagcctgcacagctacggagtggctggagctctggtggcttttaagctgcctggatgtagctttagcatctttaagaccagcgaaagaagccagcctagactgatcttttgtcacagcaagaagaagttttgggctaagcacatgtggaattttatccctttctatggaaaggctatcagaatgtatgtgggaggagtggaacacagacagctgtttacatttagccctagaaggagactgggagtgagagctacaagaaaggtgggaatctatctgctgcctaatagatgaaagcttggg

SEQ ID NO: 91 - protein, 281 aa, Artificial Sequence
  HCV.2
MGMQVQIQSLFLLLLWVPGSRGDLMGYIPLVAKFVAAWTLKAAALLFLLLADALIFCHSKKKQLFTFSPRRYLVTRHADVYLLPRRGPRLCTCGSSDLYHMWNFISGIFWAKHMWNFAKFVAAWTLKAAAILAGYGAGVYLVAYQATVGVAGALVAFKIPFYGKAIRMYVGGVEHRVLVGGVLAAFLLLADARVLPGCSFSIFAKFVAAWTLKAAAKTSERSQPRRLGVRATRKRLIVFPDLGVWMNRLIAFALSAFSLHSYLLFNILGGWVVGIYLLPNR

SEQ ID NO: 92 - DNA, 789 bp, Artificial Sequence
  Encoding sequence for HCV.2
gaattcgccgccaccatgggaatgcaggtgcagatccagagcctgtttctgctcctcctgtgggtgcccggatccagaggagatctgatgggatatatccctctggtggctaagtttgtggctgcttggacactgaaggctgctgctctgctgtttctgctgctggctgatgctctgatcttctgtcacagcaagaagaagcagctgtttacatttagcccaagaagatatctggtgacaagacacgctgatgtgtatctgctgcctagacgcggacctagactgtgtacatgtggaagctccgatctgtatcacatgtggaactttatcagcggaatcttttgggctaagcacatgtggaatttcatcctggctggatatggagctggagtgtatctggtggcttatcaggctacagtgggagtggctggagctctggtggctttcaagatcccattctatggaaaggctatcagaatgtatgtgggaggagtggaacacagagtgctggtgggaggagtgctggctgctttcctgctgctggctgatgctagagtgctgccaggatgtagctttagcatcttcaagacttccgaacgctcccagcctagaagactgggagtgagagctacaaggaagagactgatcgtgtttccagatctgggagtgtggatgaatagactgatcgctttcgctctgagcgctttcagcctgcacagctatctgctgttcaacatcctgggaggatgggtggtgggaatctatctgctgccaaacagatgaaagctt

SEQ ID NO: 93 - protein, 107 aa, Artificial Sequence
  HCV.3s1
MGMQVQIQSLFLLLLWVPGSRGYLVAYQATVAKFVAAWTLKAAALLFLLLADALIFCHSKKKYLVTRHADVLGFGAYMSKCTCGSSDLYHMWNFISGIFWAKHMWNF

SEQ ID NO: 94 - DNA, 345 bp, Artificial Sequence
  Encoding sequence for HCV.3s1
gaattcgccgccaccatgggaatgcaggtgcagatccaaagcctgtttctgctcctcctgtgggtgcccggatccagaggatacctcgtcgcctaccaggccactgtggctaaattcgtggcagcctggacactgaaagctgcagctctgctcttcctgctcctggccgatgcactcatcttctgccattccaagaaaaagtatctggtcaccagacatgctgacgtgctggggtttggcgcctacatgagcaagtgcacctgtggcagctccgacctgtatcacatgtggaactttatttctggaatcttttgggccaagcacatgtggaatttctgaaagctt

SEQ ID NO: 95 - protein, 106 aa, Artificial Sequence
  HCV.3s2
MGMQVQIQSLFLLLLWVPGSRGVLVGGVLAAAKFVAAWTLKAAAFLLLADARVLSAFSLHSYILAGYGAGVWMNRLIAFAIPFYGKAIVAGALVAFKVGIYLLPNR

SEQ ID NO: 96 - DNA, 342 bp, Artificial Sequence
  Encoding sequence for HCV.3s2
gaattcgccgccaccatgggaatgcaggtgcagatccaaagcctgtttctgctcctcctgtgggtgcccggatccagaggagtcctggtgggcggcgtcctggccgctgctaagtttgtcgctgcttggacactgaaggcagccgctttcctgctcctggcagacgccagggtgctgtctgccttcagcctccactcctacatcctcgcagggtatggcgcaggcgtgtggatgaatcggctgatcgcctttgccattccattctatgggaaagccattgtggctggcgccctggtggcattcaaggtcgggatctacctcctgcctaaccgctgaaagctt

SEQ ID NO: 97 - protein, 80 aa, Artificial Sequence
  HCV.3s2(-3)
MGMQVQIQSLFLLLLWVPGSRGVLVGGVLAAAKFVAAWTLKAAAFLLLADARVLSAFSLHSYILAGYGAGVWMNRLIAFA

SEQ ID NO: 98 - DNA, 264 bp, Artificial Sequence
  Encoding sequence for HCV.3s2(-3)
gaattcgccgccaccatgggaatgcaggtgcagatccaaagcctgtttctgctcctcctgtgggtgcccggatccagaggagtcctggtgggcggcgtcctggccgctgctaagtttgtcgctgcttggacactgaaggcagccgctttcctgctcctggcagacgccagggtgctgtctgccttcagcctccactcctacatcctcgcagggtatggcgcaggcgtgtggatgaatcggctgatcgcctttgcctgaggatcc

SEQ ID NO: 99 - protein, 130 aa, Artificial Sequence
  HCV.3s3
MGMQVQIQSLFLLLLWVPGSRGDLMGYIPLVAKFVAAWTLKAAARLGVRATRKLLFNILGGWVRMYVGGVEHRRLIVFPDLGVGVAGALVAFKLPGCSFSIFKTSERSQPRQLFTFSPRRYLLPRRGPRL

SEQ ID NO: 100 - DNA, 414 bp, Artificial Sequence
  Encoding sequence for HCV.3s3
gaattcgccgccaccatgggaatgcaggtgcagatccaaagcctgtttctgctcctcctgtgggtgcccggatccagaggagacctgatgggctacatccctctcgtggccaagtttgtggcagcttggaccctgaaggccgctgccagactgggagtgcgcgctacacggaaactcctgtttaacatcctgggagggtgggtgcggatgtacgtcggaggcgtcgagcacagaaggctcattgtctttccagatctcggcgtgggcgtcgcaggcgcactcgtggccttcaaactgccagggtgcagcttcagcattttcaagacctccgaacgctcccaacccagacagctgttcactttctctcctcggaggtatctgctgcccagacgcggacccaggctgtgaaagctt

SEQ ID NO: 101 - protein, 98 aa, Artificial Sequence
  HCV.PC3
MGMQVQIQSLFLLLLWVPGSRGLLFNILGGWVKAKFVAAWTLKAAALADGGCSGGAYRLIVFPDLGVKFWAKHMWNFIGVAGALVAFKKQLFTFSPRR

SEQ ID NO: 102 - DNA, 318 bp, Artificial Sequence
  Encoding sequence for HCV.PC3
gaattcgccgccaccatgggaatgcaggtgcagatccaaagcctgtttctgctcctcctgtgggtgcccggatccagaggactgctcttcaacatcctgggcggatgggtgaaggccaagttcgtggctgcctggaccctgaaggctgccgctctggccgacgggggatgcagcggcggagcttacaggctcattgtctttcccgatctcggagtcaaattttgggcaaagcacatgtggaatttcatcggggtggccggagccctggtcgcttttaaaaagcagctcttcaccttctccccaagacggtgaggtacc

SEQ ID NO: 103 - protein, 107 aa, Artificial Sequence
  HCV.PC4
MGMQVQIQSLFLLLLWVPGSRGRLGVRATRKKAKFVAAWTLKAAAKTSERSQPRNLPGCSFSIFNDLMGYIPLVKYLLPRRGPRLNTLCGFADLMGYRMYVGGVEHR

SEQ ID NO: 104 - DNA, 345 bp, Artificial Sequence
  Encoding sequence for HCV.PC4
gaattcgccgccaccatgggaatgcaggtgcagatccaaagcctgtttctgctcctcctgtgggtgcccggatccagaggaaggctgggcgtgagagccacccggaagaaggccaagttcgtggctgcctggaccctgaaggctgccgctaaaacaagcgagcgctcccagcccaggaacctgcctggatgctctttcagcatctttaatgacctcatggggtacattccactggtgaagtatctgctccccagacggggccctcgcctgaacactctctgtggatttgctgatctgatggggtacaggatgtatgtcggcggagtcgaacacagatgaggtacc

SEQ ID NO: 105 - protein, 308 aa, Artificial Sequence
  HCV.2431(1P)
MGMQVQIQSLFLLLLWVPGSRGVLVGGVLAAAFLLLADARVLSAFSLHSYILAGYGAGVWMNRLIAFAGAAARLGVRATRKKAAAKTSERSQPRNLPGCSFSIFNDLMGYIPLVKYLLPRRGPRLNTLCGFADLMGYRMYVGGVEHRKLLFNILGGWVKAAALADGGCSGGAYRLIVFPDLGVKFWAKHMWNFIGVAGALVAFKKQLFTFSPRRNGYLVAYQATVAAALLFLLLADALIFCHSKKKYLVTRHADVLGFGAYMSKCTCGSSDLYHMWNFISGIFWAKHMWNFKAAAAKFVAAWTLKAAA

SEQ ID NO: 106 - DNA, 948 bp, Artificial Sequence
  Encoding sequence for HCV.2431(1P)
gaattcgccgccaccatgggaatgcaggtgcagatccaaagcctgtttctgctcctcctgtgggtgcccggctccagaggagtcctggtgggcggcgtcctggcagccgctttcctgctcctggcagacgccagggtgctgtctgccttcagcctccactcctacatcctcgcagggtatggcgcaggcgtgtggatgaatcggctgatcgcctttgccggcgctgccgcaaggctgggcgtgagagccacccggaagaaggctgccgctaaaacaagcgagcgctcccagcccaggaacctgcctggatgctctttcagcatctttaatgacctcatggggtacattccactggtgaagtatctgctccccagacggggccctcgcctgaacactctctgtggatttgctgatctgatggggtacaggatgtatgtcggcggagtcgaacacagaaaactgctcttcaacatcctgggcggatgggtgaaggctgccgctctggccgacgggggatgcagcggcggagcttacaggctcattgtctttcccgatctcggagtcaaattttgggcaaagcacatgtggaatttcatcggggtggccggagccctggtcgcttttaaaaagcagctcttcaccttctccccaagacggaacggatacctcgtcgcctaccaggccactgtggctgcagctctgctcttcctgctcctggccgatgcactcatcttctgccattccaagaaaaagtatctggtcaccagacatgctgacgtgctggggtttggcgcctacatgagcaagtgcacctgtggcagctccgacctgtatcacatgtggaactttatttctggaatcttttgggccaagcacatgtggaattttaaggccgcagcagctaaattcgtggcagcctggacactgaaagcagctgcatgaggatcc

SEQ ID NO: 107 - protein, 308 aa, Artificial Sequence
  HCV.4312(1P)
MGMQVQIQSLFLLLLWVPGSRGRLGVRATRKKAAAKTSERSQPRNLPGCSFSIFNDLMGYIPLVKYLLPRRGPRLNTLCGFADLMGYRMYVGGVEHRKLLFNILGGWVKAAALADGGCSGGAYRLIVFPDLGVKFWAKHMWNFIGVAGALVAFKKQLFTFSPRRNGYLVAYQATVAAALLFLLLADALIFCHSKKKYLVTRHADVLGFGAYMSKCTCGSSDLYHMWNFISGIFWAKHMWNFKKAAAVLVGGVLAAAFLLLADARVLSAFSLHSYILAGYGAGVWMNRLIAFANAAAKFVAAWTLKAAA

SEQ ID NO: 108 - DNA, 948 bp, Artificial Sequence
  Encoding sequence for HCV.4312(1P)
gaattcgccgccaccatgggaatgcaggtgcagatccaaagcctgtttctgctcctcctgtgggtgcccggctccagaggaaggctgggcgtgagagccacccggaagaaggctgccgctaaaacaagcgagcgctcccagcccaggaacctgcctggatgctctttcagcatctttaatgacctcatggggtacattccactggtgaagtatctgctccccagacggggccctcgcctgaacactctctgtggatttgctgatctgatggggtacaggatgtatgtcggcggagtcgaacacagaaaactgctcttcaacatcctgggcggatgggtgaaggctgccgctctggccgacgggggatgcagcggcggagcttacaggctcattgtctttcccgatctcggagtcaaattttgggcaaagcacatgtggaatttcatcggggtggccggagccctggtcgcttttaaaaagcagctcttcaccttctccccaagacggaacggatacctcgtcgcctaccaggccactgtggctgcagctctgctcttcctgctcctggccgatgcactcatcttctgccattccaagaaaaagtatctggtcaccagacatgctgacgtgctggggtttggcgcctacatgagcaagtgcacctgtggcagctccgacctgtatcacatgtggaactttatttctggaatcttttgggccaagcacatgtggaattttaagaaagccgctgcagtcctggtgggcggcgtcctggcagccgctttcctgctcctggcagacgccagggtgctgtctgccttcagcctccactcctacatcctcgcagggtatggcgcaggcgtgtggatgaatcggctgatcgcctttgccaatgctgcagctaaattcgtggcagcctggacactgaaagcagctgcatgaggatcc

SEQ ID NO: 109 - protein, 123 aa, Artificial Sequence
  AOSI.K
MGMQVQIQSLFLLLLWVPGSRGHTLWKAGILYKAKFVAAWTLKAAAFLPSDFFPSVKFLLSLGIHLYMDDVVLGVGLSRYVARLFLLTRILTISTLPETTVVRRQAFTFSPTYKWLSLLVPFV

SEQ ID NO: 110 - DNA, 372 bp, Artificial Sequence
  Encoding sequence for AOSI.K
atgggaatgcaggtgcagatccagagcctgtttctgctcctcctgtgggtgcccgggtccagaggacacaccctgtggaaggccggaatcctgtataaggccaagttcgtggctgcctggaccctgaaggctgccgctttcctgcctagcgatttctttcctagcgtgaagttcctgctgtccctgggaatccacctgtatatggatgacgtggtgctgggagtgggactgtccaggtacgtggctaggctgttcctgctgaccagaatcctgaccatctccaccctgccagagaccaccgtggtgaggaggcaggccttcacctttagccctacctataagtggctgagcctgctggtgccctttgtgtga

SEQ ID NO: 111 - protein, 206 aa, Artificial Sequence
  HBV.1
MGMQVQIQSLFLLLLWVPGSRGHTLWKAGILYKAKFVAAWTLKAAAFLPSDFFPSVFLLSLGIHLYMDDVVLGVGLSRYVARLFLLTRILTISTLPETTVVRRQAFTFSPTYKWLSLLVPFVIPIPSSWAFTPARVTGGVFKVGNFTGLYLPSDFFPSVTLWKAGILYKNVSIPWTHKLVVDFSQFSRSAICSVVRRALMPLYACI

SEQ ID NO: 112 - DNA, 621 bp, Artificial Sequence
  Encoding sequence for HBV.1
atgggaatgcaggtgcagatccagagcctgtttctgctcctcctgtgggtgcccgggtccagaggacacaccctgtggaaggccggaatcctgtataaggccaagttcgtggctgcctggaccctgaaggctgccgctttcctgcctagcgatttctttcctagcgtgttcctgctgtccctgggaatccacctgtatatggatgacgtggtgctgggagtgggactgtccaggtacgtggctaggctgttcctgctgaccagaatcctgaccatctccaccctgccagagaccaccgtggtgaggaggcaggccttcacctttagccctacctataagtggctgagcctgctggtgccctttgtgatccctatccctagctcctgggctttcaccccagccagggtgaccggaggagtgtttaaggtgggaaacttcaccggcctgtatctgcccagcgatttctttcctagcgtgaccctgtggaaggccgggatcctgtacaagaatgtgtccatcccttggacccacaagctggtggtggacttttcccagttcagcagatccgctatctgctccgtggtgaggagagctctgatgccactgtatgcctgtatctga

SEQ ID NO: 113 - protein, 219 aa, Artificial Sequence
  HBV.2
MGMQVQIQSLFLLLLWVPGSRGHTLWKAGILYKAKFVAAWTLKAAAFLPSDFFPSVNFLLSLGIHLYMDDVVLGVGLSRYVARLFLLTRILTISTLPETTVVRRQAFTFSPTYKGAAAWLSLLVPFVNIPIPSSWAFKTPARVTGGVFKVGNFTGLYNLPSDFFPSVKTLWKAGILYKNVSIPWTHKGAALVVDFSQFSRNSAICSVVRRALMPLYACI

SEQ ID NO: 114 - DNA, 660 bp, Artificial Sequence
  Encoding sequence for HBV.2
atgggaatgcaggtgcagatccagagcctgtttctgctcctcctgtgggtgcccgggtccagaggacacaccctgtggaaggccggaatcctgtataaggccaagttcgtggctgcctggaccctgaaggctgccgctttcctgcctagcgatttctttcctagcgtgaacttcctgctgtccctgggaatccacctgtatatggatgacgtggtgctgggagtgggactgtccaggtacgtggctaggctgttcctgctgaccagaatcctgaccatctccaccctgccagagaccaccgtggtgaggaggcaggccttcacctttagccctacctataagggagccgctgcctggctgagcctgctggtgccctttgtgaatatccctatccctagctcctgggctttcaagaccccagccagggtgaccggaggagtgtttaaggtgggaaacttcaccggcctgtataacctgcccagcgatttctttcctagcgtgaagaccctgtggaaggccggaatcctgtacaagaatgtgtccatcccttggacccacaagggagccgctctggtggtggacttttcccagttcagcagaaattccgctatctgctccgtggtgaggagagctctgatgccactgtatgcctgtatctga

SEQ ID NO: 115 - protein, 168 aa, Artificial Sequence
  PfCTL.1
MQVQIQSLFLLLLWVPGSRGILSVSSFLFVNAAAQTNFKSLLRNLPSENERGYKAAALLACAGLAYKKAAAAKFVAAWTLKAAAKAFMKAVCVEVNAAASFLFVEALFNATPYAGEPAPFKAAAKYKLATSVLKAGVSENIFLKNAAAYFILVNLLIKAGLLGVVSTV

SEQ ID NO: 116 - DNA, 513 bp, Artificial Sequence
  Encoding sequence for PfCTL.1
atgggaatgcaggtgcagatccagagcctgtttctgctcctcctgtgggtgcccggatccagaggaatcctgagcgtgtcctctttcctgtttgtcaacgccgctgcacagaccaatttcaagagcctcctgaggaacctcccctccgagaacgaaagaggctacaaagccgctgcactgctcgcctgcgctggactggcctataagaaagccgctgcagccaagttcgtggccgcttggacactgaaggccgctgcaaaagcctttatgaaggctgtctgtgtggaggtcaatgccgctgcatctttcctgtttgtggaggccctctttaacgctactccttacgcaggggaaccagcccccttcaaggccgctgcaaaatataagctggcaaccagcgtgctgaaggctggcgtgtccgagaatatttttctgaaaaacgccgctgcatacttcatcctggtgaatctgctcattaaggccggactcctgggggtggtctctacagtgtga

SEQ ID NO: 117 - protein, 157 aa, Artificial Sequence
  PfCTL.2
MQVQIQSLFLLLLWVPGSRGFVEALFQEYNAAAKYLVIVFLINALACAGLAYKKFYFILVNLLKAALFFIIFNKNAAAKFVAAWTLKAAAKFILVNLLIFHNFQDEENIGIYKLPYGRTNLKAAAVLLGGVGLVLNFLIFFDLFLVKAVLAGLLGVV

SEQ ID NO: 118 - DNA, 480 bp, Artificial Sequence
  Encoding sequence for PfCTL.2
atgggaatgcaggtgcagatccagagcctgtttctgctcctcctgtgggtgcccggatccagaggattcgtggaggccctgtttcaggaatacaacgccgctgcaaagtatctcgtcatcgtgttcctgatcaatgctctggcatgcgccggcctcgcttacaaaaagttttacttcattctggtcaacctgctcaaggccgctctgttctttatcattttcaataaaaacgccgcagctaagtttgtggccgcatggaccctgaaggccgctgcaaaattcatcctcgtgaatctgctcatttttcacaacttccaagacgaggaaaatatcggaatttataagctgccctacgggaggacaaacctgaaagccgctgcagtcctgctcggcggagtggggctggtgctcaattttctgatcttctttgatctgttcctggtgaaggccgtcctggccggcctgctcggagtcgtgtga

SEQ ID NO: 119 - protein, 169 aa, Artificial Sequence
  PfCTL.3
MQVQIQSLFLLLLWVPGSRGVFLIFFDLFLNAAAPSDGKCNLYKAAAVTCGNGIQVRKLFHIFDGDNEIKAHVLSHNSYEKNYYGKQENWYSLKKILSVFFLANAAAKFIKSLFHIFKAAALYISFYFIKAKFVAAWTLKAAAKAAAYYIPHQSSLKAAAGLIMVLSFL

SEQ ID NO: 120 - DNA, 516 bp, Artificial Sequence
  Encoding sequence for PfCTL.3
atgggaatgcaggtgcagatccagagcctgtttctgctcctcctgtgggtgcccggatccagaggagtgttcctgatcttctttgacctgttcctgaacgccgctgcacccagcgatggcaagtgcaatctctacaaggccgctgcagtgacctgtggaaacgggattcaggtcaggaaactctttcacatcttcgacggcgataacgagatcaaggcccatgtgctgtcccacaattcttatgaaaaaaactactatggaaagcaagagaattggtacagcctgaagaaaattctgtccgtgttctttctcgccaacgccgctgcaaagtttatcaagtctctgttccatattttcaaggccgctgcactctacatcagcttctattttattaaagccaaatttgtggccgcttggacactgaaggccgctgcaaaagccgctgcatactatatccctcaccagagctccctgaaggccgctgcagggctgatcatggtgctctctttcctgtga

SEQ ID NO: 121 - protein, 456 aa, Artificial Sequence
  PfCTL/HTL(N)
MQVQIQSLFLLLLWVPGSRGSSVFNVVNSSIGLIMVLSFLGPGPGLYISFYFILVNLLIFHINGKIIKNSEGPGPGPDSIQDSLKESRKLSGPGPGVLAGLLGVVSTVLLGGVGLVLGPGPGLPSENERGYYIPHQSSLGPGPGQTNFKSLLRNLGVSENIFLKGPGPGFQDEENIGIYGPGPGKYLVIVFLIFFDLFLVGPGPGKFIKSLFHIFDGDNEIGPGPGKSKYKLATSVLAGLLGPGPGLPYGKTNLGPGPGRHNWVNHAVPLAMKLIGPGPGMRKLAILSVSSFLFVEALFQEYGPGPGVTCGNGIQVRGPGPGMNYYGKQENWYSLKKGPGPGPSDGKCNLYADSAWENVKNVIGPFMKAVCVEVGPGPGKILSVFFLALFFIIFNKGPGPGHVLSHNSYEKGPGPGKYKIAGGIAGGLALLACAGLAYKFVVPGAATPYAGEPAPF

SEQ ID NO: 122 - DNA, 1385 bp, Artificial Sequence
  Encoding sequence for PfCTL/HTL(N)
atgggaatgcaggtgcagatccagagcctgtttctgctcctcctgtgggtgcccggatccagaggaagtagtgtgttcaatgttgtgaactcatcaattggtctgatcatggtgctgagctttctcgggccagggccaggattatatatttctttctacttcatccttgtcaacctgttaatattccacattaacggcaaaataataaagaacagtgaaggccctgggcctgggcctgactcgatccaggattctctaaaagaatcgaggaagctctccggaccaggccctggtgtactcgccgggttgctgggagtagttagcacagtgctgttaggaggcgtcggcctcgtcttaggacctggaccaggtctgccgtccgaaaacgaaagaggatactacatacctcaccagagcagcctcggcccaggccccggacaaaccaatttcaaatccctcttgcgaaatctaggagtgagcgagaacatatttcttaaaggacccggtcccggctttcaggacgaggagaatataggtatttacggtccaggacctggaaaatacctagtgatcgtattcctaattttttttgacctatttctggtgggcccaggtcccggaaagttcattaaatcactcttccacatttttgacggagataacgagataggacccggtcccgggaaatcaaagtacaaactagccacttcagtgctggccggccttctagggccgggcccagggctcccctatggaaagacaaatcttggccccggtccaggacggcacaactgggtgaatcatgcggttccattggccatgaaactaatcgggcccggtccaggcatgcgcaaacttgcaattctaagcgtaagttcatttctgttcgtagaggcactgtttcaagaatatggcccaggacctggcgtcacatgtgggaatgggatccaggtgagaggaccgggacctggtatgaactattacggtaaacaggaaaattggtactccctgaaaaagggtccaggccccggcccctcagatggtaagtgcaacctgtatgctgactcagcatgggagaacgtaaaaaatgtaataggcccattcatgaaggcagtttgtgtcgaagtcggaccaggcccaggaaaaatactttctgtcttcttcctagctctcttcttcatcatcttcaacaagggaccagggccaggtcacgtgttatcccataactcttatgaaaaagggccaggacctgggaaatacaaaatcgcaggagggatcgccggcgggctagcgctccttgcctgcgcaggcttggcttacaaattcgttgtaccaggagctgcaacaccctatgcaggagaacctgccccattttgaagatctgc

SEQ ID NO: 123 - protein, 419 aa, Artificial Sequence
  Pf33
MGMQVQIQSLFLLLLWVPGSRGFMKAVCVEVNVTCGNGIQVRKGLIMVLSFLNAALFHIFDGDNEIKAALLACAGLAYKKSFLFVEALFNAAPSDGKCNLYKAAQTNFKSLLRNLPSENERGYKAAGVSENIFLKNAAAYFILVNLLIKAAAILSVSSFLFVNTPYAGEPAPFKAAAKYKLATSVLKAAVFLIFFDLFLNYYIPHQSSLKAAGLLGNVSTVGAVLLGGVGLVLNLACAGLAYKKAKFIKSLFHIFKAAFYFILVNLLKAFLIFFDLFLVKALFFIIFNKNYYGKQENWYSLKFVEALFQEYNAAAKFVAAWTLKAAAKILSVFFLANAVLAGLLGNVNFQDEENIGIYKAAALYISFYFIKAFILVNLLIFHNAALPYGRTNLKAAHVLSHNSYEKNAAAKYLVIVFLI

SEQ ID NO: 124 - DNA, 1269 bp, Artificial Sequence
  Encoding sequence for Pf33
gccgccaccatgggaatgcaggtgcagatccagagcctgtttctgctcctcctgtgggtgcccggatccagaggatttatgaaagctgtctgtgtagaggtgaatgtaacatgcggtaacggaattcaggtgagaaagggactcatcatggtactcagctttctgaacgcagccctgttccacatctttgacggagacaatgaaatcaaagccgcattgctcgcctgtgccggactagcctataaaaagagtttccttttcgttgaagcactatttaacgcagcacccagtgacggtaaatgcaacctatataaagcagctcagactaatttcaaaagcctgttaagaaatctgccctcagagaatgaaaggggttacaaagccgccggcgtgtccgagaatattttcctgaagaacgccgctgcttattttatactcgtgaatctactcataaaggcagccgcaatcctttcagtgtccagctttctgtttgttaacacaccatatgcgggcgagccggctcctttcaaggctgcagcaaaatacaagcttgccacatcagtattgaaagcagctgtgtttttgatattctttgatctttttttaaactactacatacctcatcagtctagtcttaaagcagccgggctactggggaacgtctctactgtgggggccgtcttacttggaggagttggcctcgtgttgaacctcgcgtgcgcaggtctggcctacaaaaaagcgaaattcatcaagtctctgttccacatttttaaagccgcattctatttcatactagtgaaccttctcaaagctttcctgatcttcttcgatctattcctcgtaaaagcgctattcttcattatctttaacaaaaattattacggcaagcaagaaaattggtactcactcaagtttgtagaagctctgttccaggaatacaacgccgctgctaaattcgttgcagcttggaccctgaaagcagctgcaaagatcctatcggtcttctttctcgctaatgccgtattagcaggacttctaggcaacgtgaactttcaagacgaagagaatataggcatctacaaagccgcagcactgtacatttcattctacttcatcaaggccttcatactggtcaaccttctgatatttcataatgcagcactgccatatgggagaaccaacttgaaagcggcccacgtgttgagccacaactcctacgagaagaacgccgccgcgaaatatctcgtcattgtcttcctgatttga

SEQ ID NO: 125 - protein, 180 aa, Artificial Sequence
  TB.1
MQVQIQSLFLLLLWVPGSRGRMSRVTTFTVKALVLLMLPVVNLMIGTAAAVVKALVLLMLPVGAGLMTAVYLVGAAAMALLRLPVKRMFAANLGVNSLYFGGICVGRLPLVLPAVNAAAAKFVAAWTLKAAAKAAARLMIGTAAAGFVVALIPLVNAMTYAAPLFVGAAAAMALLRLPLV

SEQ ID NO: 126 - DNA, 543 bp, Artificial Sequence
  Encoding sequence for TB.1
atgcaggtgcagatccagagcctgtttctgctcctcctgtgggtgcccggatccagaggaaggatgagcagagtgaccacattcactgtcaaggccctggtgctcctgatgctccccgtcgtgaacctgatgatcggcaccgctgcagccgtcgtgaaagctctcgtcctgctcatgctccctgtgggagcagggctgatgacagccgtgtacctggtcggcgctgcagccatggccctcctgcggctgccagtgaagcgcatgtttgctgcaaatctgggagtcaactccctctatttcgggggcatttgcgtgggaaggctgcccctcgtgctgcctgctgtgaatgcagccgctgccaaatttgtcgccgcttggactctgaaggcagccgctaaggccgctgcaagactgatgatcgggaccgccgctgccggcttcgtggtcgccctgattcccctggtgaacgccatgacatacgcagctcctctgtttgtgggagccgctgcagccatggctctcctgcggctgccactggtgtga

SEQ ID NO: 127 - protein, 148 aa, Artificial Sequence
  BCL A2 #90
MQVQIQSLFLLLLWVPGSRGIMIGHLVGVNRLLQETELVNAKVAEIVHFLNAKVFGSLAFVNAYLSGANLNVGAAYLQLVFGIEVNAAAKFVAAWTLKAAAKAAAVVLGVVFGINSMPPPGTRVNAAAATVGIMIGVNAKLCPVQLWV

SEQ ID NO: 128 - DNA, 447 bp, Artificial Sequence
  Encoding sequence for BCL A2 #90
atgcaggtgcagatccagagcctgtttctgctcctcctgtgggtgcccgggtccagaggaattatgatcggccatctggtgggcgtcaacagactgctgcaggaaaccgagctggtgaatgccaaggtggccgaaattgtgcactttctcaacgcaaaggtgtttggttccctggcttttgtcaatgcctatctgagcggcgctaacctcaacgtcggagccgcctacctccagctggtcttcggcatcgaggtcaacgctgctgcaaaattcgtggcagcttggaccctcaaggctgcagcaaaggctgccgccgtcgtgctcggagtggtgttcgggatcaactctatgccacctcccgggactagggtcaatgctgccgccgcaacagtgggaatcatgattggggtgaatgccaaactgtgcccagtgcaactgtgggtgtga

SEQ ID NO: 129 - protein, 144 aa, Artificial Sequence
  BCL A2 #88
MQVQIQSLFLLLLWVPGSRGVVLGVVFGINAAAAKFVAAWTLKAAAKVAEIVHFLNAYLSGANLNVGAAYLQLVFGIEVNIMIGHLVGVNRLLQETELVNAKVFGSLAFVNAKLCPVQLWVNAAAATVGIMIGVNSMPPPGTRV

SEQ ID NO: 130 - DNA, 435 bp, Artificial Sequence
  Encoding sequence for BCL A2 #88
atgcaggtgcagatccagagcctgtttctgctcctcctgtgggtgcccgggtccagaggagtcgtgctgggagtcgtcttcggcattaatgccgccgctgcaaagttcgtggctgcctggaccctgaaggccgcagctaaagtggcagagatcgtgcactttctgaacgcctacctgagcggagcaaatctgaacgtcggcgctgcctatctgcagctcgtgtttggaattgaagtgaacatcatgattggacatctggtgggcgtgaacaggctgctccaggaaactgagctggtcaacgctaaagtgttcgggtctctcgcctttgtgaacgctaagctctgccccgtccaactctgggtcaatgccgcagccgctacagtggggatcatgatcggcgtgaactccatgcctccaccagggaccagagtgtga

SEQ ID NO: 131 - protein, 147 aa, Artificial Sequence
  BCL A2 #63
MQVQIQSLFLLLLWVPGSRGKLCPVQLWVNAAAATVGIMIGVNIMIGHLVGVNRLLQETELVNAKVAEIVHFLNAKVFGSLAFVNAYLSGANLNVGAAYLQLVFGIEVNAAAKFVAAWTLKAAAKAAAVVLGVVFGINSMPPPGTRV

SEQ ID NO: 132 - DNA, 450 bp, Artificial Sequence
  Encoding sequence for BCL A2 #63
atgcaggtgcagatccagagcctgtttctgctcctcctgtgggtgcccgggtccagaggaaagctctgccccgtgcaactgtgggtcaacgccgccgccgcaaccgtcggcattatgatcggggtgaacatcatgatcggacacctggtcggcgtgaacaggctgctgcaggagacagaactggtcaatgccaaggtggctgaaattgtccatttcctgaatgccaaagtgttcggctctctcgctttcgtgaacgcttatctgagcggagctaacctcaacgtgggggccgcatacctccagctcgtctttgggattgaggtgaatgccgcagctaaatttgtcgctgcctggaccctgaaggcagcagccaaggctgccgcagtggtgctgggagtggtgtttggaatcaattccatgcctccaccaggcactagagtgtgaggatcc

SEQ ID NO: 133 - protein, 183 aa, Artificial Sequence
  Prostate 1
LTFFWLDRSVKAAAVLVHPQWVLTVKAAALLQERGVAYIKAALLLSIALSVNPLVCNGVLQGVKAAIMYSAHDTTVKAAAFLTPKKLQCVNAMMNDQLMFLNAGLPSIPVHPVKAAALGTTCYVGAAILLWQPIPVNFLRPRSLQCVKAFLTLSVTWIGVNALLYSLVHNLGAATLMSAMTNL

SEQ ID NO: 134 - DNA, 648 bp, Artificial Sequence
  Encoding sequence for Prostate 1
atgcaggtgcagatccagagcctgtttctgctcctcctgtgggtgcccgggtccagaggattgacatttttttggctggatagatcggttaaggctgcagccgtgcttgttcatccccagtgggtcttgaccgtaaaggctgccgcgctgctacaagaaagaggggtcgcatacatcaaagctgctctcctcttgagtattgcgctaagtgtaaacccgctagtttgtaatggggtgttacaaggtgtgaaagcggcgattatgtacagtgcccacgacactaccgtaaaagcagccgctttcctgaccccaaaaaaactccaatgcgtgaacgcaatgatgaatgatcagctgatgtttttaaacgctggcttaccttctataccggttcatccagtcaaggccgcggcattgggtacgacgtgttatgttggagcagcgatacttctttggcagcccataccagtaaattttttaagacctagatccttacaatgcgtcaaagcattccttacactctcagtaacttggatcggagtcaatgctctgctatatagcctcgtacacaacttgggcgcggccacacttatgagtgcaatgacgaatttagctaagttcgtggcggcctggactctaaaggccgcagca

SEQ ID NO: 135 - protein, 322 aa, Artificial Sequence
  HIV-1043
MEKVYLAWVPAHKGIGGGPGPGQKQITKIQNFRVYYRGPGPGWEFVNTPPLVKLWYQGPGPGYRKILRQRKIDRLIDGPGPGQHLLQLTVWGIKQLQGPGPGGEIYKRWIILGLNKIVRMYGPGPGQGQMVHQAISPRTLNGPGPGIKQFINMWQEVGKAMYGPGPGWAGIKQEFGIPYNPQGPGPGKTAVQMAVFIHNFKRGPGPGSPAIFQSSMTKILEPGPGPGEVNIVTDSQYALGIIGPGPGHSNWRAMASDFNLPPGPGPGAETFYVDGAANRETKGPGPGGAVVIQDNSDIKVVPGPGPGFRKYTAFTIPSINNE

SEQ ID NO: 136 - DNA, 969 bp, Artificial Sequence
  Encoding sequence for HIV-1043
atggagaaggtgtacctggcctgggttccagcccacaaaggcatcgggggagggcccggacctgggcagaaacagatcaccaagatccagaacttccgggtatactaccggggacctggtccaggttgggagtttgtgaacacaccacccttagtaaagctctggtaccagggccccggtcccggataccgtaaaatcctgaggcaaagaaagatagatcgcctcattgatggcccgggcccaggccagcaccttctgcagcttacagtgtggggaattaaacagctgcaggggccgggccccgggggggaaatttataaaaggtggatcattctgggtctgaacaagatcgtccgcatgtatggccctggacccggacaggggcagatggtccaccaagcaatcagccctcgaaccttgaatggaccgggcccaggaatcaagcaattcattaacatgtggcaagaagttggtaaggctatgtacggtcccggccctggatgggcagggataaaacaggagtttggaatcccttacaatccccagggtcctgggccaggtaaaacggcagtgcagatggccgtgttcattcataattttaagcggggccctggacctggcagcccagctatatttcaaagttcgatgaccaaaatcttggagcccggcccagggccgggcgaagtgaacattgtcacagattctcagtatgccctcggcatcatagggcccggaccagggcattccaattggcgcgccatggcgtctgactttaatctacctcctgggccaggccctggcgcggaaactttctatgtggacggcgctgcaaacagggagactaagggacccggacccggcggcgctgtagtcattcaggacaactcagacatcaaggtggttcccggtccaggccccgggttcagaaagtataccgccttcactattccgtccatcaacaatgagtga

SEQ ID NO: 137 - protein, 340 aa, Artificial Sequence
  HIV-1043 PADRE
MEKVYLAWVPAHKGIGGGPGPGQKQITKIQNFRVYYRGPGPGWEFVNTPPLVKLWYQGPGPGYRKILRQRKIDRLIDGPGPGQHLLQLTVWGIKQLQGPGPGGEIYKRWIILGLNKIVRMYGPGPGQGQMVHQAISPRTLNGPGPGIKQFINMWQEVGKAMYGPGPGWAGIKQEFGIPYNPQGPGPGKTAVQMAVFIHNFKRGPGPGSPAIFQSSMTKILEPGPGPGEVNIVTDSQYALGIIGPGPGHSNWRAMASDFNLPPGPGPGAETFYVDGAANRETKGPGPGGAVVIQDNSDIKVVPGPGPGFRKYTAFTIPSINNEGPGPGAKFVAAWTLKAAA

SEQ ID NO: 138 - DNA, 1023 bp, Artificial Sequence
  Encoding sequence for HIV-1043 PADRE
atggagaaggtgtacctggcctgggttccagcccacaaaggcatcgggggagggcccggacctgggcagaaacagatcaccaagatccagaacttccgggtatactaccggggacctggtccaggttgggagtttgtgaacacaccacccttagtaaagctctggtaccagggccccggtcccggataccgtaaaatcctgaggcaaagaaagatagatcgcctcattgatggcccgggcccaggccagcaccttctgcagcttacagtgtggggaattaaacagctgcaggggccgggccccgggggggaaatttataaaaggtggatcattctgggtctgaacaagatcgtccgcatgtatggccctggacccggacaggggcagatggtccaccaagcaatcagccctcgaaccttgaatggaccgggcccaggaatcaagcaattcattaacatgtggcaagaagttggtaaggctatgtacggtcccggccctggatgggcagggataaaacaggagtttggaatcccttacaatccccagggtcctgggccaggtaaaacggcagtgcagatggccgtgttcattcataattttaagcggggccctggacctggcagcccagctatatttcaaagttcgatgaccaaaatcttggagcccggcccagggccgggcgaagtgaacattgtcacagattctcagtatgccctcggcatcatagggcccggaccagggcattccaattggcgcgccatggcgtctgactttaatctacctcctgggccaggccctggcgcggaaactttctatgtggacggcgctgcaaacagggagactaagggacccggacccggcggcgctgtagtcattcaggacaactcagacatcaaggtggttcccggtccaggccccgggttcagaaagtataccgccttcactattccgtccatcaacaatgagggccccggcccaggtgccaagttcgtggctgcctggaccctgaaggctgccgcttga

SEQ ID NO: 139 - protein, 75 aa, Artificial Sequence
  HIV 75 mer
EKVYLAWVPAHKGIGGPGPGQGQMVHQAISPRTLNGPGPGSPAIFQSSMTKILEPGPGPGFRKYTAFTIPSINNE

SEQ ID NO: 140 - DNA, 228 bp, Artificial Sequence
  Encoding sequence for HIV 75mer
gagaaggtgtacctggcctgggtgcctgcccacaagggaatcggaggacctggccctggacagggacagatggtgcaccaggccatcagccctaggaccctgaacggacctggacctggaagccctgccatcttccagagcagcatgaccaagatcctggagcccggacctggacctggattcaggaagtacaccgccttcaccatccccagcatcaacaacgagtga

SEQ ID NO: 141 - protein, 276 aa, Artificial Sequence
  PfHTL
MQVQIQSLFLLLLWVPGSRGRHNWVNHAVPLAMKLIGPGPGKCNLYADSAWENVKNGPGPGKSKYKLATSVLAGLLGPGPGQTNFKSLLRNLGVSEGPGPGSSVFNVVNSSIGLIMGPGPGVKNVIGPFMKAVCVEGPGPGMNYYGKQENWYSLKKGPGPGGLAYKFVVPGAATPYGPGPGPDSIQDSLKESRKLNGPGPGLLIFHINGKIIKNSEGPGPGAGLLGNVSTVLLGGVGPGPGKYKIAGGIAGGLALLGPGPGMRKLAILSVSSFLFV

SEQ ID NO: 142 - DNA, 837 bp, Artificial Sequence
  Encoding sequence for PfHTL
atgggaatgcaggtgcagatccagagcctgtttctgctcctcctgtgggtgcccggatccagaggaaggcacaactgggtgaatcatgctgtgcccctggctatgaagctgatcggccctggaccagggaaatgcaacctctacgcagacagcgcctgggagaacgtcaagaatggccccggacctgggaaatccaagtataagctcgctacctctgtgctggcaggcctgctcggaccaggccccggacagacaaatttcaaaagcctgctcagaaacctgggagtgtccgaggggcctggcccaggatctagcgtctttaatgtggtcaactcctctattgggctcatcatgggacccggacctggggtgaaaaatgtcattggcccattcatgaaggccgtgtgtgtcgaaggacccgggcctggcatgaactactatggaaagcaagaaaattggtacagcctgaagaaaggccctgggccaggcggactggcttacaagtttgtggtcccaggggcagccactccctatgggcctgggccaggccccgattccatccaggactctctcaaagagagccggaaactgaacggacccgggcctggactgctcattttccacatcaatggcaaaattatcaagaacagcgagggacctgggccaggcgccggactgctggggaacgtgtccaccgtcctgctcggcggagtggggcccggccctgggaagtacaagatcgctggagggatcgcaggcggactggccctcctgggcccaggaccagggatgcgcaaactggctattctctctgtctccagctttctgtttgtgtga

SEQ ID NO: 143 - protein, 9 aa, Artificial sequence
  Sequence for epitope
VLAEAMSQV

SEQ ID NO: 144 - protein, 9 aa, Artificial sequence
  Sequence for epitope
MTNNPPIPV

SEQ ID NO: 145 - protein, 10 aa, Artificial sequence
  Sequence for epitope
MASDFNLPPV

SEQ ID NO: 146 - protein, 9 aa, Artificial sequence
  Sequence for epitope
KLVGKLNWA

SEQ ID NO: 147 - protein, 9 aa, Artificial sequence
  Sequence for epitope
LVGPTPVNI

SEQ ID NO: 148 - protein, 9 aa, Artificial sequence
  Sequence for epitope
ILKEPVHGV

SEQ ID NO: 149 - protein, 9 aa, Artificial sequence
  Sequence for epitope
KAACWWAGI

SEQ ID NO: 150 - protein, 10 aa, Artificial sequence
  Sequence for epitope
KMIGGIGGFI

SEQ ID NO: 151 - protein, 9 aa, Artificial sequence
  Sequence for epitope
RAMASDFNL

SEQ ID NO: 152 - protein, 9 aa, Artificial sequence
  Sequence for epitope
TLNFPISPI

SEQ ID NO: 153 - protein, 9 aa, Artificial sequence
  Sequence for epitope
KLTPLCVTL

SEQ ID NO: 154 - protein, 9 aa, Artificial sequence
  Sequence for epitope
LLQLTVWGI

SEQ ID NO: 155 - protein, 10 aa, Artificial sequence
  Sequence for epitope
SLLNATDIAV

SEQ ID NO: 156 - protein, 9 aa, Artificial sequence
  Sequence for epitope
LTFGWCFKL

SEQ ID NO: 157 - protein, 9 aa, Artificial sequence
  Sequence for epitope
AIIRILQQL

SEQ ID NO: 158 - protein, 9 aa, Artificial sequence
  Sequence for epitope
RILQQLLFI

SEQ ID NO: 159 - protein, 10 aa, Artificial sequence
  Sequence for epitope
QMAVFIHNFK

SEQ ID NO: 160 - protein, 11 aa, Artificial sequence
  Sequence for epitope
KVYLAWVPAHK

SEQ ID NO: 161 - protein, 10 aa, Artificial sequence
  Sequence for epitope
KIQNFRVYYR

SEQ ID NO: 162 - protein, 9 aa, Artificial sequence
  Sequence for epitope
AIFQSSMTK

SEQ ID NO: 163 - protein, 10 aa, Artificial sequence
  Sequence for epitope
VTIKIGGQLK

SEQ ID NO: 164 - protein, 10 aa, Artificial sequence
  Sequence for epitope
TTLFCASDAK

SEQ ID NO: 165 - protein, 11 aa, Artificial sequence
  Sequence for epitope
VTVYYGVPVWK

SEQ ID NO: 166 - protein, 10 aa, Artificial sequence
  Sequence for epitope
QVPLRPMTYK

SEQ ID NO: 167 - protein, 9 aa, Artificial sequence
  Sequence for epitope
VMIVWQVDR

SEQ ID NO: 168 - protein, 10 aa, Artificial sequence
  Sequence for epitope
QMVHQAISPR

SEQ ID NO: 169 - protein, 10 aa, Artificial sequence
  Sequence for epitope
YPLASLRSLF

SEQ ID NO: 170 - protein, 9 aa, Artificial sequence
  Sequence for epitope
HPVHAGPIA

SEQ ID NO: 171 - protein, 9 aa, Artificial sequence
  Sequence for epitope
FPISPIETV

SEQ ID NO: 172 - protein, 11 aa, Artificial sequence
  Sequence for epitope
IPYNPQSQGVV

SEQ ID NO: 173 - protein, 9 aa, Artificial sequence
  Sequence for epitope
IPIHYCAPA

SEQ ID NO: 174 - protein, 9 aa, Artificial sequence
  Sequence for epitope
CPKVSFEPI

SEQ ID NO: 175 - protein, 9 aa, Artificial sequence
  Sequence for epitope
FPVRPQVPL

SEQ ID NO: 176 - protein, 8 aa, Artificial sequence
  Sequence for epitope
VPLQLPPL

SEQ ID NO: 177 - protein, 10 aa, Artificial sequence
  Sequence for epitope
EVNIVTDSQY

SEQ ID NO: 178 - protein, 9 aa, Artificial sequence
  Sequence for epitope
FRDYVDRFY

SEQ ID NO: 179 - protein, 10 aa, Artificial sequence
  Sequence for epitope
VIYQYMDDLY

SEQ ID NO: 180 - protein, 10 aa, Artificial sequence
  Sequence for epitope
VTVLDVGDAY

SEQ ID NO: 181 - protein, 9 aa, Artificial sequence
  Sequence for epitope
IYQEPFKNL

SEQ ID NO: 182 - protein, 9 aa, Artificial sequence
  Sequence for epitope
PYNTPVFAI

SEQ ID NO: 183 - protein, 9 aa, Artificial sequence
  Sequence for epitope
TYQIYQEPF

SEQ ID NO: 184 - protein, 10 aa, Artificial sequence
  Sequence for epitope
YWQATWIPEW

SEQ ID NO: 185 - protein, 9 aa, Artificial sequence
  Sequence for epitope
IWGCSGKLI

SEQ ID NO: 186 - protein, 9 aa, Artificial sequence
  Sequence for epitope
RYLKDQQLL

SEQ ID NO: 187 - protein, 10 aa, Artificial sequence
  Sequence for epitope
VWKEATTTLF

SEQ ID NO: 188 - protein, 9 aa, Artificial sequence
  Sequence for epitope
IYETYGDTW

SEQ ID NO: 189 - protein, 9 aa, Artificial sequence
  Sequence for epitope
PYNEWTLEL

SEQ ID NO: 190 - protein, 15 aa, Artificial sequence
  Sequence for epitope
KRWIILGLNKIVRMY

SEQ ID NO: 191 - protein, 15 aa, Artificial sequence
  Sequence for epitope
WEFVNTPPLVKLWYQ

SEQ ID NO: 192 - protein, 15 aa, Artificial sequence
  Sequence for epitope
QKQITKIQNFRVYYR

SEQ ID NO: 193 - protein, 15 aa, Artificial sequence
  Sequence for epitope
KVYLAWVPAHKGIGG

SEQ ID NO: 194 - protein, 15 aa, Artificial sequence
  Sequence for epitope
GEIYKRWIILGLNKI

SEQ ID NO: 195 - protein, 15 aa, Artificial sequence
  Sequence for epitope
EKVYLAWVPAHKGIG

SEQ ID NO: 196 - protein, 15 aa, Artificial sequence
  Sequence for epitope
QHLLQLTVWGIKQLQ

SEQ ID NO: 197 - protein, 15 aa, Artificial sequence
  Sequence for epitope
QGQMVHQAISPRTLN

SEQ ID NO: 198 - protein, 15 aa, Artificial sequence
  Sequence for epitope
SPAIFQSSMTKILEP

SEQ ID NO: 199 - protein, 16 aa, Artificial sequence
  Sequence for epitope
IKQFINMWQEVGKAMY

SEQ ID NO: 200 - protein, 15 aa, Artificial sequence
  Sequence for epitope
FRKYTAFTIPSINNE

SEQ ID NO: 201 - protein, 15 aa, Artificial sequence
  Sequence for epitope
HSNWRAMASDFNLPP

SEQ ID NO: 202 - protein, 15 aa, Artificial sequence
  Sequence for epitope
KTAVQMAVFIHNFKR

SEQ ID NO: 203 - protein, 15 aa, Artificial sequence
  Sequence for epitope
YRKILRQRKIDRLID

SEQ ID NO: 204 - protein, 15 aa, Artificial sequence
  Sequence for epitope
WAGIKQEFGIPYNPQ

SEQ ID NO: 205 - protein, 15 aa, Artificial sequence
  Sequence for epitope
EVNIVTDSQYALGII

SEQ ID NO: 206 - protein, 15 aa, Artificial sequence
  Sequence for epitope
AETFYVDGAANRETK

SEQ ID NO: 207 - protein, 15 aa, Artificial sequence
  Sequence for epitope
GAVVIQDNSDIKVVP

SEQ ID NO: 208 - protein, 10 aa, Artificial sequence
  Sequence for epitope
LLFNILGGWV

SEQ ID NO: 209 - protein, 9 aa, Artificial sequence
  Sequence for epitope
FLLLADARV

SEQ ID NO: 210 - protein, 9 aa, Artificial sequence
  Sequence for epitope
YLVAYQATV

SEQ ID NO: 211 - protein, 10 aa, Artificial sequence
  Sequence for epitope
RLIVFPDLGV

SEQ ID NO: 212 - protein, 9 aa, Artificial sequence
  Sequence for epitope
DLMGYIPLV

SEQ ID NO: 213 - protein, 9 aa, Artificial sequence
  Sequence for epitope
WMNRLIAFA

SEQ ID NO: 214 - protein, 9 aa, Artificial sequence
  Sequence for epitope
VLVGGVLAA

SEQ ID NO: 215 - protein, 9 aa, Artificial sequence
  Sequence for epitope
HMWNFISGI

SEQ ID NO: 216 - protein, 9 aa, Artificial sequence
  Sequence for epitope
ILAGYGAGV

SEQ ID NO: 217 - protein, 10 aa, Artificial sequence
  Sequence for epitope
YLLPRRGPRL

SEQ ID NO: 218 - protein, 9 aa, Artificial sequence
  Sequence for epitope
LLFLLLADA

SEQ ID NO: 219 - protein, 9 aa, Artificial sequence
  Sequence for epitope
YLVTRHADV

SEQ ID NO: 220 - protein, 9 aa, Artificial sequence
  Sequence for epitope
KTSERSQPR

SEQ ID NO: 221 - protein, 9 aa, Artificial sequence
  Sequence for epitope
RLGVRATRK

SEQ ID NO: 222 - protein, 9 aa, Artificial sequence
  Sequence for epitope
QLFTFSPRR

SEQ ID NO: 223 - protein, 10 aa, Artificial sequence
  Sequence for epitope
RMYVGGVEHR

SEQ ID NO: 224 - protein, 9 aa, Artificial sequence
  Sequence for epitope
LIFCHSKKK

SEQ ID NO: 225 - protein, 10 aa, Artificial sequence
  Sequence for epitope
GVAGALVAFK

SEQ ID NO: 226 - protein, 9 aa, Artificial sequence
  Sequence for epitope
VAGALVAFK

SEQ ID NO: 227 - protein, 9 aa, Artificial sequence
  Sequence for epitope
LGFGAYMSK

SEQ ID NO: 228 - protein, 9 aa, Artificial sequence
  Sequence for epitope
LPGCSFSIF

SEQ ID NO: 229 - protein, 9 aa, Artificial sequence
  Sequence for epitope
LSAFSLHSY

SEQ ID NO: 230 - protein, 9 aa, Artificial sequence
  Sequence for epitope
CTCGSSDLY

SEQ ID NO: 231 - protein, 9 aa, Artificial sequence
  Sequence for epitope
LTDPSHITA

SEQ ID NO: 232 - protein, 11 aa, Artificial sequence
  Sequence for epitope
LTCGFADLMGY

SEQ ID NO: 233 - protein, 11 aa, Artificial sequence
  Sequence for epitope
LADGGCSGGAY

SEQ ID NO: 234 - protein, 9 aa, Artificial sequence
  Sequence for epitope
FWAKHMWNF

SEQ ID NO: 235 - protein, 9 aa, Artificial sequence
  Sequence for epitope
RMILMTHFF

SEQ ID NO: 236 - protein, 8 aa, Artificial sequence
  Sequence for epitope
VMGSSYGF

SEQ ID NO: 237 - protein, 10 aa, Artificial sequence
  Sequence for epitope
FWAKHMWNFI

SEQ ID NO: 238 - protein, 9 aa, P. falciparum
FMKAVCVEV

SEQ ID NO: 239 - protein, 9 aa, P. falciparum
GLLGVVSTV

SEQ ID NO: 240 - protein, 10 aa, P. falciparum
ILSVSSFLFV

SEQ ID NO: 241 - protein, 9 aa, P. falciparum
QTNFKSLLR

SEQ ID NO: 242 - protein, 9 aa, P. falciparum
GVSENIFLK

SEQ ID NO: 243 - protein, 10 aa, P. falciparum
LLACAGLAYK

SEQ ID NO: 244 - protein, 10 aa, P. falciparum
TPYAGEPAPF

SEQ ID NO: 245 - protein, 9 aa, P. falciparum
LPSENERGY

SEQ ID NO: 246 - protein, 9 aa, P. falciparum
KYKLATSVL

SEQ ID NO: 247 - protein, 9 aa, P. falciparum
SFLFVEALF

SEQ ID NO: 248 - protein, 9 aa, P. falciparum
YFILVNLLI

SEQ ID NO: 249 - protein, 10 aa, P. falciparum
FLIFFDLFLV

SEQ ID NO: 250 - protein, 9 aa, P. falciparum
VLAGLLGVV

SEQ ID NO: 251 - protein, 10 aa, P. falciparum
VLLGGVGLVL

SEQ ID NO: 252 - protein, 9 aa, P. falciparum
LACAGLAYK

SEQ ID NO: 253 - protein, 9 aa, P. falciparum
ALFFIIFNK

SEQ ID NO: 254 - protein, 10 aa, P. falciparum
FILVNLLIFH

SEQ ID NO: 255 - protein, 8 aa, P. falciparum
LPYGRTNL

SEQ ID NO: 256 - protein, 9 aa, P. falciparum
FVEALFQEY

SEQ ID NO: 257 - protein, 10 aa, P. falciparum
FQDEENIGIY

SEQ ID NO: 258 - protein, 9 aa, P. falciparum
FYFILVNLL

SEQ ID NO: 259 - protein, 9 aa, P. falciparum
KYLVIVFLI

SEQ ID NO: 260 - protein, 9 aa, P. falciparum
GLIMVLSFL

SEQ ID NO: 261 - protein, 9 aa, P. falciparum
KILSVFFLA

SEQ ID NO: 262 - protein, 10 aa, P. falciparum
VTCGNGIQVR

SEQ ID NO: 263 - protein, 10 aa, P. falciparum
HVLSHNSYEK

SEQ ID NO: 264 - protein, 9 aa, P. falciparum
PSDGKCNLY

SEQ ID NO: 265 - protein, 9 aa, P. falciparum
YYIPHQSSL

SEQ ID NO: 266 - protein, 10 aa, P. falciparum
KFIKSLFHIF

SEQ ID NO: 267 - protein, 10 aa, P. falciparum
VFLIFFDLFL

SEQ ID NO: 268 - protein, 11 aa, P. falciparum
LFHIFDGDNEI

SEQ ID NO: 269 - protein, 11 aa, P. falciparum
YYGKQENWYSL

SEQ ID NO: 270 - protein, 8 aa, P. falciparum
LYISFYFI

SEQ ID NO: 271 - protein, 15 aa, P. falciparum
MRKLAILSVSSFLFV

SEQ ID NO: 272 - protein, 15 aa, P. falciparum
MNYYGKQENWYSLKK

SEQ ID NO: 273 - protein, 15 aa, P. falciparum
SSVFNVVNSSIGLIM

SEQ ID NO: 274 - protein, 16 aa, P. falciparum
RHNWVNHAVPLAMKLI

SEQ ID NO: 275 - protein, 15 aa, P. falciparum
PDSIQDSLKESRKLN

SEQ ID NO: 276 - protein, 15 aa, P. falciparum
KCNLYADSAWENVKN

SEQ ID NO: 277 - protein, 15 aa, P. falciparum
VKNVIGPFMKAVCVE

SEQ ID NO: 278 - protein, 15 aa, P. falciparum
KYKIAGGIAGGLALL

SEQ ID NO: 279 - protein, 15 aa, P. falciparum
GLAYKFVVPGAATPY

SEQ ID NO: 280 - protein, 15 aa, P. falciparum
KSKYKLATSVLAGLL

SEQ ID NO: 281 - protein, 15 aa, P. falciparum
AGLLGNVSTVLLGGV

SEQ ID NO: 282 - protein, 15 aa, P. falciparum
LLIFHINGKIIKNSE

SEQ ID NO: 283 - protein, 15 aa, P. falciparum
QTNFKSLLRNLGVSE

SEQ ID NO: 284 - protein, 10 aa, Artificial sequence
  Sequence for epitope
FLPSDFFPSV

SEQ ID NO: 285 - protein, 9 aa, Artificial sequence
  Sequence for epitope
FLLTRILTI

SEQ ID NO: 286 - protein, 9 aa, Artificial sequence
  Sequence for epitope
WLSLLVPFV

SEQ ID NO: 287 - protein, 9 aa, Artificial sequence
  Sequence for epitope
GLSRYVARL

SEQ ID NO: 288 - protein, 9 aa, Artificial sequence
  Sequence for epitope
YMDDVVLGV

SEQ ID NO: 289 - protein, 10 aa, Artificial sequence
  Sequence for epitope
ILRGTSFVYV

SEQ ID NO: 290 - protein, 9 aa, Artificial sequence
  Sequence for epitope
FLLSLGIHL

SEQ ID NO: 291 - protein, 9 aa, Artificial sequence
  Sequence for epitope
ALMPLYACI

SEQ ID NO: 292 - protein, 10 aa, Artificial sequence
  Sequence for epitope
GLSPTVWLSV

SEQ ID NO: 293 - protein, 11 aa, Artificial sequence
  Sequence for epitope
STLPETTVVRR

SEQ ID NO: 294 - protein, 11 aa, Artificial sequence
  Sequence for epitope
HTLWKAGILYK

SEQ ID NO: 295 - protein, 10 aa, Artificial sequence
  Sequence for epitope
TLWKAGILYK

SEQ ID NO: 296 - protein, 10 aa, Artificial sequence
  Sequence for epitope
LVVDFSQFSR

SEQ ID NO: 297 - protein, 9 aa, Artificial sequence
  Sequence for epitope
NVSIPWTHK

SEQ ID NO: 298 - protein, 9 aa, Artificial sequence
  Sequence for epitope
SAICSVVRR

SEQ ID NO: 299 - protein, 9 aa, Artificial sequence
  Sequence for epitope
KVGNFTGLY

SEQ ID NO: 300 - protein, 10 aa, Artificial sequence
  Sequence for epitope
QAFTFSPTYK

SEQ ID NO: 301 - protein, 9 aa, Artificial sequence
  Sequence for epitope
LPSDFFPSV

SEQ ID NO: 302 - protein, 9 aa, Artificial sequence
  Sequence for epitope
IPIPSSWAF

SEQ ID NO: 303 - protein, 10 aa, Artificial sequence
  Sequence for epitope
TPARVTGGVF

SEQ ID NO: 304 - protein, 10 aa, Artificial sequence
  Sequence for epitope
RMSRVTTFTV

SEQ ID NO: 305 - protein, 10 aa, Artificial sequence
  Sequence for epitope
ALVLLMLPVV

SEQ ID NO: 306 - protein, 10 aa, Artificial sequence
  Sequence for epitope
LMIGTAAAVV

SEQ ID NO: 307 - protein, 9 aa, Artificial sequence
  Sequence for epitope
ALVLLMLPV

SEQ ID NO: 308 - protein, 9 aa, Artificial sequence
  Sequence for epitope
GLMTAVYLV

SEQ ID NO: 309 - protein, 8 aa, Artificial sequence
  Sequence for epitope
MALLRLPV

SEQ ID NO: 310 - protein, 9 aa, Artificial sequence
  Sequence for epitope
RMFAANLGV

SEQ ID NO: 311 - protein, 9 aa, Artificial sequence
  Sequence for epitope
SLYFGGICV

SEQ ID NO: 312 - protein, 9 aa, Artificial sequence
  Sequence for epitope
RLPLVLPAV

SEQ ID NO: 313 - protein, 9 aa, Artificial sequence
  Sequence for epitope
RLMIGTAAA

SEQ ID NO: 314 - protein, 9 aa, Artificial sequence
  Sequence for epitope
FVVALIPLV

SEQ ID NO: 315 - protein, 9 aa, Artificial sequence
  Sequence for epitope
MTYAAPLFV

SEQ ID NO: 316 - protein, 10 aa, Artificial sequence
  Sequence for epitope
AMALLRLPLV

SEQ ID NO: 317 - protein, 9 aa, Artificial sequence
  Sequence for epitope
KLCPVQLWV

SEQ ID NO: 318 - protein, 9 aa, Artificial sequence
  Sequence for epitope
ATVGIMIGV

SEQ ID NO: 319 - protein, 9 aa, Artificial sequence
  Sequence for epitope
IMIGHLVGV

SEQ ID NO: 320 - protein, 9 aa, Artificial sequence
  Sequence for epitope
RLLQETELV

SEQ ID NO: 321 - protein, 9 aa, Artificial sequence
  Sequence for epitope
KVAEIVHFL

SEQ ID NO: 322-324 - skipped (3 entries)

SEQ ID NO: 325 - protein, 9 aa, Artificial sequence
  Sequence for epitope
VVLGVVFGI

SEQ ID NO: 326 - protein, 9 aa, Artificial sequence
  Sequence for epitope
SMPPPGTRV

SEQ ID NO: 327 - protein, 10 aa, Artificial sequence
  Sequence for epitope
LTFFWLDRSV

SEQ ID NO: 328 - protein, 9 aa, Artificial sequence
  Sequence for epitope
TLMSAMTNL

SEQ ID NO: 329 - protein, 10 aa, Artificial sequence
  Sequence for epitope
IMYSAHDTTV

SEQ ID NO: 330 - protein, 10 aa, Artificial sequence
  Sequence for epitope
GLPSIPVHPV

SEQ ID NO: 331 - protein, 10 aa, Artificial sequence
  Sequence for epitope
LLQERGVAYI

SEQ ID NO: 332 - protein, 9 aa, Artificial sequence
  Sequence for epitope
LLYSLVHNL

SEQ ID NO: 333 - protein, 9 aa, Artificial sequence
  Sequence for epitope
MMNDQLMFL

SEQ ID NO: 334 - protein, 11 aa, Artificial sequence
  Sequence for epitope
FLTLSVTWIGV

SEQ ID NO: 335 - protein, 8 aa, Artificial sequence
  Sequence for epitope
ALGTTCYV

SEQ ID NO: 336 - protein, 10 aa, Artificial sequence
  Sequence for epitope
FLTPKKLQCV

SEQ ID NO: 337 - protein, 9 aa, Artificial sequence
  Sequence for epitope
LLLSIALSV

SEQ ID NO: 338 - protein, 11 aa, Artificial sequence
  Sequence for epitope
VLVHPQWVLTV

SEQ ID NO: 339 - protein, 10 aa, Artificial sequence
  Sequence for epitope
FLRPRSLQCV

SEQ ID NO: 340 - protein, 11 aa, Artificial sequence
  Sequence for epitope
PLVCNGVLQGV

SEQ ID NO: 341 - protein, 10 aa, Artificial Sequence
  Motif Specification 206
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XXXXNXXXXL

SEQ ID NO: 342 - protein, 10 aa, Artificial Sequence
  Motif Specification 206
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XXXXNXXXXI

SEQ ID NO: 343 - protein, 10 aa, Artificial Sequence
  Motif Specification 206
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XXXXNXXXXM

SEQ ID NO: 344 - protein, 10 aa, Artificial Sequence
  Motif Specification 206
  VARIANT (1): Xaa = Any Amino Acid
  VARIANT (2): Xaa = Any Amino Acid
  VARIANT (3): Xaa = Any Amino Acid
  VARIANT (4): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XXXXNXXXXV

SEQ ID NO: 345 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXK

SEQ ID NO: 346 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXR

SEQ ID NO: 347 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XLXXXXXXY

SEQ ID NO: 348 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXK

SEQ ID NO: 349 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXR

SEQ ID NO: 350 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XMXXXXXXY

SEQ ID NO: 351 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XVXXXXXXK

SEQ ID NO: 352 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XVXXXXXXR

SEQ ID NO: 353 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XVXXXXXXY

SEQ ID NO: 354 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XTXXXXXXK

SEQ ID NO: 355 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XTXXXXXXR

SEQ ID NO: 356 - protein, 9 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
XTXXXXXXY

SEQ ID NO: 357 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XLXXXXXXXK

SEQ ID NO: 358 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XLXXXXXXXR

SEQ ID NO: 359 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XLXXXXXXXY

SEQ ID NO: 360 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XMXXXXXXXK

SEQ ID NO: 361 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XMXXXXXXXR

SEQ ID NO: 362 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XMXXXXXXXY

SEQ ID NO: 363 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XVXXXXXXXK

SEQ ID NO: 364 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XVXXXXXXXR

SEQ ID NO: 365 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XVXXXXXXXY

SEQ ID NO: 366 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XTXXXXXXXK

SEQ ID NO: 367 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XTXXXXXXXR

SEQ ID NO: 368 - protein, 10 aa, Artificial Sequence
  Motif Specification 206
  VARIANT (1): Xaa = Any Amino Acid
  VARIANT (3): Xaa = Any Amino Acid
  VARIANT (4): Xaa = Any Amino Acid
  VARIANT (5): Xaa = Any Amino Acid
  VARIANT (6): Xaa = Any Amino Acid
  VARIANT (7): Xaa = Any Amino Acid
  VARIANT (8): Xaa = Any Amino Acid
  VARIANT (9): Xaa = Any Amino Acid
XTXXXXXXXY

SEQ ID NO: 369 - protein, 9 aa, Artificial sequence
  HBV CTL epitope
HPAAMPHLL

SEQ ID NO: 370 - protein, 11 aa, Artificial sequence
  HBV CTL epitope
WMMWYWGPSLY

SEQ ID NO: 371 - protein, 10 aa, Artificial sequence
  HBV CTL epitope
DLLDTASALY

SEQ ID NO: 372 - protein, 11 aa, Artificial sequence
  HBV CTL epitope
LTFGRETVLEY

SEQ ID NO: 373 - protein, 10 aa, Artificial sequence
  HBV CTL epitope
LSLDVSAAFY

SEQ ID NO: 374 - protein, 10 aa, Artificial sequence
  HBV CTL epitope
SWPKFAVPNL

SEQ ID NO: 375 - protein, 11 aa, Artificial sequence
  HBV CTL epitope
RFSWLSLLVPF

SEQ ID NO: 376 - protein, 9 aa, Artificial sequence
  HBV CTL epitope
EYLVSFGVW

SEQ ID NO: 377 - protein, 9 aa, Artificial sequence
  HBV CTL epitope
KYTSFPWLL

SEQ ID NO: 378 - protein, 10 aa, Artificial sequence
  HBV CTL epitope
FPHCLAFSYM

SEQ ID NO: 379 - protein, 11 aa, Artificial sequence
  HBV CTL epitope
YPALMPLYACI

SEQ ID NO: 380 - protein, 8 aa, Artificial sequence
  HBV CTL epitope
ASFCGSPY

SEQ ID NO: 381 - protein, 10 aa, Artificial sequence
  HBV CTL epitope
ILLLCLIFLL

SEQ ID NO: 382 - protein, 10 aa, Artificial sequence
  HBV CTL epitope
RWMCLRRFII

SEQ ID NO: 383 - protein, 10 aa, Artificial sequence
  HBV CTL epitope
LWFHISCLTF

SEQ ID NO: 384 - protein, 333 aa, Artificial sequence
  Amino acid sequence of HBV-2A epitope
MGMQVQIQSLFLLLLWVPGSRGHTLWKAGILYKAKFVAAWTLKAAAFLPSDFFPSVNFLLSLGIHLYMDDVVLGVGLSRYVARLFLLTRILTISTLPETTVVRRQAFTFSPTYKGAAAWLSLLVPFVNIPIPSSWAFKTPARVTGGVFKVGNFTGLYNLPSDFFPSVKTLWKAGILYKNVSIPWTHKGAALVVDFSQFSRNSAICSVVRRKAWMMWYWGPSLYKKYTSFPWLLNAHPAAMPHLLKAAADLLDTASALYNAAARFSWLSLLVPFNAASWPKFAVPNLKLTFGRETVLEYKALSLDVSAAFYGAAEYLVSFGVWGAALMPLYACI

SEQ ID NO: 385 - DNA, 1002 bp, Artificial sequence
  Nucleotide sequence encoding amino acid sequence of HBV-2A epitope
atgggaatgcaggtgcagatccagagcctgtttctgctcctcctgtgggtgcccgggtccagaggacacaccctgtggaaggccggaatcctgtataaggccaagttcgtggctgcctggaccctgaaggctgccgctttcctgcctagcgatttctttcctagcgtgaacttcctgctgtccctgggaatccacctgtatatggatgacgtggtgctgggagtgggactgtccaggtacgtggctaggctgttcctgctgaccagaatcctgaccatctccaccctgccagagaccaccgtggtgaggaggcaggccttcacctttagccctacctataagggagccgctgcctggctgagcctgctggtgccctttgtgaatatccctatccctagctcctgggctttcaagaccccagccagggtgaccggaggagtgtttaaggtgggaaacttcaccggcctgtataacctgcccagcgatttctttcctagcgtgaagaccctgtggaaggccggaatcctgtacaagaatgtgtccatcccttggacccacaagggagccgctctggtggtggacttttcccagttcagcagaaatagcgccatctgttcggtcgtgagaaggaaagcctggatgatgtggtactggggtcctagtctgtataagaagtacacctcattcccatggctcttgaatgcccatcccgctgcaatgccacacctgcttaaagctgcggcggatctgctggacacagcctcagctttatataatgctgcagcaagattctcctggttgtctctcttagtgcccttcaacgcagcttcctggccaaaatttgccgttccgaacctgaagctcacttttggaagagagacagtacttgaatacaaagcactaagccttgacgtgtcagcagccttctacggagcagcagaatatctagtatcttttggggtctggggcgcagccctcatgcctctatacgcctgcatttga

SEQ ID NO: 386 - protein, 333 aa, Artificial sequence
  Amino acid sequence of HBV-2B epitope
MGMQVQIQSLFLLLLWVPGSRGHTLWKAGILYKAKFVAAWTLKAAAFLPSDFFPSVNFLLSLGIHLYMDDVVLGVGLSRYVARLFLLTRILTISTLPETTVVRRQAFTFSPTYKGAAAWLSLLVPFVNIPIPSSWAFKTPARVTGGVFKVGNFTGLYNLPSDFFPSVKTLWKAGILYKNVSIPWTHKGAALVVDFSQFSRNSAICSVVRRKEYLVSFGVWGLSLDVSAAFYNAAAKYTSFPWLLNAHPAAMPHLLKAAADLLDTASALYNSWPKFAVPNLKLTFGRETVLEYKAAWMMWYWGPSLYKAAARFSWLSLLVPFGAAALMPLYACI

SEQ ID NO: 387 - DNA, 1002 bp, Artificial sequence
  Nucleotide sequence encoding amino acid sequence of HBV-2B epitope
atgggaatgcaggtgcagatccagagcctgtttctgctcctcctgtgggtgcccgggtccagaggacacaccctgtggaaggccggaatcctgtataaggccaagttcgtggctgcctggaccctgaaggctgccgctttcctgcctagcgatttctttcctagcgtgaacttcctgctgtccctgggaatccacctgtatatggatgacgtggtgctgggagtgggactgtccaggtacgtggctaggctgttcctgctgaccagaatcctgaccatctccaccctgccagagaccaccgtggtgaggaggcaggccttcacctttagccctacctataagggagccgctgcctggctgagcctgctggtgccctttgtgaatatccctatccctagctcctgggctttcaagaccccagccagggtgaccggaggagtgtttaaggtgggaaacttcaccggcctgtataacctgcccagcgatttctttcctagcgtgaagaccctgtggaaggccggaatcctgtacaagaatgtgtccatcccttggacccacaagggagccgctctggtggtggacttttcccagttcagcagaaattcagcaatttgttcggtggtgagaagaaaggaatatcttgtttcatttggcgtctgggggctgtcactggatgtaagtgcggcattttacaatgccgccgcaaaatatacaagcttcccatggctcctaaacgcacacccagctgcaatgccgcatctactgaaagcagccgctgacctcttagacactgcctccgctctgtacaactcttggcccaagtttgccgtgcctaatctcaagttgaccttcggtagagagacagtcttagaatacaaagcggcctggatgatgtggtactggggaccctctctgtataaagccgctgcaaggttctcctggcttagccttctcgtaccattcggagcagctgccctaatgcctttgtacgcatgcatctga

SEQ ID NO: 388 - protein, 295 aa, Artificial sequence
  Amino acid sequence of HBV-21A epitope
MGMQVQIQSLFLLLLWVPGSRGSWPKFAVPNLKAAAAKFVAAWTLKAAAKSTLPETTVVRRKHPAAMPHLLKAAAHTLWKAGILYKKAFLLTRILTIGALSLDVSAAFYNAAAKYTSFPWLLNAAARFSWLSLLVPFNAATPARVTGGVFKAAEYLVSFGVWGAAAYMDDVVLGVNDLLDTASALYNAAAFPHCLAFSYMKAAAWMMWYWGPSLYKAASAICSVVRRKNFLLSLGIHLNIPIPSSWAFKAAWLSLLVPFVNAFLPSDFFPSVKLTFGRETVLEYKQAFTFSPTYK

SEQ ID NO: 389 - DNA, 888 bp, Artificial sequence
  Nucleotide sequence encoding amino acid sequence of HBV-21A epitope
atgggaatgcaggtgcagatccagagcctgtttctgctcctcctgtgggtgcccgggtccagaggatcttggcctaaattcgcagtgccaaaccttaaagccgcggctgctaagttcgtagctgcctggacactaaaggccgccgctaagagcacactgccagagaccaccgtggtccggcgaaagcatccagccgcaatgccccacttgctcaaagcagccgcccacactctttggaaggctgggatattgtacaagaaagccttccttctgaccaggatattaactatcggagctctgtcactcgacgtttctgctgccttctacaacgcggcggcaaaatacactagctttccatggctactcaacgcagccgccagattttcttggctatcactactggtgccatttaatgcagcaacacctgctagagtgactggcggcgtctttaaagcagccgagtacttggtgagctttggcgtctggggtgcagcggcatatatggatgatgtagtgttaggggtgaacgacctcctggacacagccagtgcgctgtacaatgcagctgcattcccgcattgcctagccttcagttatatgaaagcagcagcctggatgatgtggtactggggaccgtccctttataaagcagcttcagcaatctgttccgttgtgaggagaaaaaactttttactctccctcggtattcacctgaacattcccatcccttcctcatgggcattcaaagccgcttggctgagtctactcgtacctttcgttaatgcatttctgcccagcgactttttcccctcggtaaaactgacattcggacgcgaaacagtccttgaatataagcaggccttcacgttctcaccaacctataaatga

SEQ ID NO: 390 - protein, 296 aa, Artificial sequence
  Amino acid sequence of HBV-21B epitope
MGMQVQIQSLFLLLLWVPGSRGYMDDVVLGVNAAAEYLVSFGVWNDLLDTASALYGAAHTLWKAGILYKKAFLPSDFFPSVKAFPHCLAFSYMKAARFSWLSLLVPFNAASWPKFAVPNLKAAAQAFTFSPTYKNAAASAICSVVRRKAFLLTRILTINIPIPSSWAFKAAWMMWYWGPSLYKAAATPARVTGGVFKAANFLLSLGIHLNLTFGRETVLEYKHPAAMPHLLKAASTLPETTVVRRKWLSLLVPFVNAAAAKFVAAWTLKAAAKLSLDVSAAFYNAAAKYTSFPWLL

SEQ ID NO: 391 - DNA, 891 bp, Artificial sequence
  Nucleotide sequence encoding amino acid sequence of HBV-21B epitope
atgggaatgcaggtgcagatccagagcctgtttctgctcctcctgtgggtgcccgggtccagaggatacatggatgacgttgtgttaggcgttaatgcagccgcagaatatctcgtgtcattcggcgtctggaacgacctgttggacactgcatctgctctgtacggtgcagcccataccctgtggaaggccggaatcctctacaaaaaggcattcctacctagcgacttttttccttcagtgaaagccttcccacattgcctagcattctcgtatatgaaagcggctaggttctcatggcttagtcttctagtacctttcaatgccgcctcctggcccaaattcgccgtaccaaatctaaaagcggccgcgcaggcctttacattctctccgacttataaaaatgcagcagcctccgctatttgtagcgtcgtgcgccgaaaggccttcctgctaacccggattttgacgataaacatccccatcccttctagctgggctttcaaagcagcatggatgatgtggtactggggtcccagcttatacaaagctgcggcaaccccagcaagagtgacagggggcgtgtttaaggccgccaacttcctcctgagtctcggaatacacctgaacttaacctttgggagagagacagtactggagtataaacacccagcagctatgccgcacctactcaaagccgcttcaacactcccagaaacaactgtagtgaggagaaaatggctctccctgcttgtcccatttgtcaacgccgccgccgctaagtttgtggccgcttggacacttaaggctgcagcaaagttgtcacttgatgttagtgcagcgttctataacgcagctgcaaaatacacttcctttccctggctgctgtga

SEQ ID NO: 392 - protein, 403 aa, Artificial sequence
  Amino acid sequence of HBV-30B epitope
MGMQVQIQSLFLLLLWVPGSRGFLLTRILTINAAASWPKFAVPNLKAAAHTLWKAGILYKKADLLDTASALYNQAFTFSPTYKGAAANVSIPWTHKGAAAFLLSLGIHLNIPIPSSWAFKAAALWFHISCLTFKAAAILLLCLIFLLNAAAYPALMPLYACINAHPAAMPHLLKAAASFCGSPYKAAGLSRYVARLNKYTSFPWLLNFLPSDFFPSVKAFPHCLAFSYMKAEYLVSFGVWNAALTFGRETVLEYKAAALPSDFFPSVKAYMDDVVLGVNLVVDFSQFSRNAAARWMCLRRFIINAARFSWLSLLVPFNAATPARVTGGVFKAAWLSLLVPFVNSAICSVVRRKAKFVAAWTLKAAAKWMMWYWGPSLYKAASTLPETTVVRRKLSLDVSAAFY

SEQ ID NO: 393 - DNA, 1215 bp, Artificial sequence
  Nucleotide sequence encoding amino acid sequence of HBV-30B epitope
atgggaatgcaggtccagatacagagcttgttcctcctcctgctttgggtccccggatcaaggggtttcctcctaacccgcatcctgacaattaacgccgcagcctcctggccaaaatttgccgtgccaaatctcaaggcagctgcacacacactatggaaagcagggatactgtacaagaaagccgatctgctagacacagcgtctgcgttgtacaaccaggcttttactttctctcctacatataaaggcgcagctgcaaacgtgagtatcccttggacgcacaaaggagccgctgccaacttcttactgtccctgggcatccatctaaatatccctattccttcatcctgggcatttaaagcagccgccttatggttccacataagttgtctgaccttcaaagccgcagcaatcctgctcctttgcctcattttcttactaaacgccgctgcctatccagctcttatgccattgtacgcatgtatcaacgcccaccccgcagcaatgccccacctccttaaagctgccgccagtttctgcggttctccttataaagcagcagggctgtccagatacgtagctaggctaaacaagtataccagcttcccctggttacttaatttcctgccgtcagatttctttccatcagttaaggccttccctcattgtctggcctttagctacatgaaggctgaatatttggtatccttcggcgtgtggaatgcggcactgacatttggaagggagacagtgctcgagtacaaagccgccgcactaccctcggacttcttcccatcggtcaaagcttacatggacgatgtagtcctcggcgttaacttagtagtggacttttctcaattttccagaaacgcagcggccagatggatgtgccttcggcgttttataataaacgccgctcgattcagctggctatcactcctagttccatttaatgcagctacacccgcacgggtgacaggtggagttttcaaggcagcgtggctttcactgcttgtgccatttgtgaactcagctatttgctcagtagtgagaaggaaggcaaaattcgtcgctgcctggactctcaaagctgccgcaaagtggatgatgtggtattggggaccgagcttgtacaaagcggcctctactctgccagaaactaccgtagtgagaagaaaactgagcctggacgtcagcgcggcattctactga

SEQ ID NO: 394 - protein, 403 aa, Artificial sequence
  Amino acid sequence of HBV-30C epitope
MGMQVQIQSLFLLLLWVPGSRGFLLSLGIHLNAAAKYTSFPWLLNAAARFSWLSLLVPFNAAFPHCLAFSYMKAALVVDFSQFSRGAILLLCLIFLLNAAAHTLWKAGILYKKAWMMWYWGPSLYKAYPALMPLYACIGAAAWLSLLVPFVNFLLTRILTINIPIPSSWAFKAAAEYLVSFGVWNLPSDFFPSVKFLPSDFFPSVKDLLDTASALYNSWPKFAVPNLKAAASAICSVVRRKLSLDVSAAFYNAAAKFVAAWTLKAAAKAANVSIPWTHKGAAGLSRYVARLNAAASTLPETTVVRRKHPAAMPHLLKAAARWMCLRRFIINASFCGSPYKAAYMDDVVLGVNALWFHISCLTFKAAATPARVTGGVFKAAALTFGRETVLEYKQAFTFSPTYK

SEQ ID NO: 395 - DNA, 1212 bp, Artificial sequence
  Nucleotide sequence encoding amino acid sequence of HBV-30C epitope
atgggaatgcaggtgcaaatacagtctctcttccttttgcttctctgggttccaggatcacggggcttcttgcttagcttgggcatccacctaaatgctgctgcaaaatacacatcttttccttggctccttaatgccgccgctaggttttcatggctgagtctgctagtacctttcaatgcggctttcccacattgcctagcttttagctatatgaaagctgctttagtcgtggacttttcacagtttagcagaggagcaatcctgctgctatgtctgatattccttctaaacgcagcagcccacacactctggaaagctggtatcctttacaagaaagcctggatgatgtggtattggggacccagcctctacaaagcataccctgccctgatgccactatacgcatgcattggcgcggcagcctggttatcccttttagtaccgtttgtcaactttctattaaccagaatcctgacgattaatattccgatcccaagttcctgggcattcaaagcagccgcggagtatctggtttcatttggcgtatggaacctgccaagcgacttctttccttctgttaagttcctcccctccgatttctttccatcggtgaaagacctccttgataccgcgagcgctctgtacaactcgtggccaaaattcgcagttccaaacctaaaagccgccgccagtgccatttgttccgtggtaaggagaaaattatcactcgacgtgtccgcagcattttataacgctgctgcaaagtttgtcgcagcatggacattgaaggctgcagcgaaagcagcaaatgtatcaataccctggacccacaagggtgcagccgggctgtctaggtatgtggcgaggctaaacgccgccgcctcaacactgcctgagactactgtcgtgagacgcaaacaccctgccgcaatgccccacctgctgaaagcagccgcacgatggatgtgcctcagaagattcataataaacgcttctttctgtgggtcaccctacaaagccgcttacatggacgatgtggtcctcggagtgaatgccctctggttccatatcagctgcctgacattcaaggcagccgccacccccgctcgtgtgacaggaggtgtcttcaaagccgcggcactgactttcggtcgggaaactgtattggaatataagcaggccttcacattctccccaacatacaagtga

SEQ ID NO: 396 - protein, 410 aa, Artificial sequence
  Amino acid sequence of HBV-CL epitope
MQVQIQSLFLLLLWVPGSRGFLLSLGIHLNAAAKYTSFPWLLNAAARFSWLSLLVPFNAAFPHCLAFSYMKAALVVDFSQFSRGAILLLCLIFLLNAAAHTLWKAGILYKKAWMMWYWGPSLYKAYPALMPLYACIGAAAWLSLLVPFVNFLLTRILTINAAAIPIPSSWAFKAAAEYLVSFGVWNLPSDFFPSVKAAAFLPSDFFPSVKAAADLLDTASALYNSWPKFAVPNLKAAASAICSVVRRKLSLDVSAAFYNAAAKFVAAWTLKAAAKAANVSIPWTHKGAAGLSRYVARLNAAASTLPETTVVRRKHPAAMPHLLKAAARWMCLRRFIINASFCGSPYKAAYMDDVVLGVNALWFHISCLTFKAAATPARVTGGVFKAAALTFGRETVLEYKQAFTFSPTYK

SEQ ID NO: 397 - DNA, 1239 bp, Artificial sequence
  Nucleotide sequence encoding amino acid sequence of HBV-CL epitope
atgggaatgcaggtgcaaatacagtctctcttccttttgcttctctgggttccaggatcacggggcttcttgcttagcttgggcatccacctaaatgctgctgcaaaatacacatcttttccttggctccttaatgccgccgctaggttttcatggctgagtctgctagtacctttcaatgcggctttcccacattgcctagcttttagctatatgaaagctgctttagtcgtggacttttcacagtttagcagaggagcaatcctgctgctatgtctgatattccttctaaacgcagcagcccacacactctggaaagctggtatcctttacaagaaagcctggatgatgtggtattggggacccagcctctacaaagcataccctgccctgatgccactatacgcatgcattggcgcggcagcctggttatcccttttagtaccgtttgtcaactttctattaaccagaatcctgacgattaatgctgccgccattccgatcccaagttcctgggcattcaaagcagccgcggagtatctggtttcatttggcgtatggaacctgccaagcgacttctttccttctgttaaggccgctgctttcctcccctccgatttctttccatcggtgaaagccgctgccgacctccttgataccgcgagcgctctgtacaactcgtggccaaaattcgcagttccaaacctaaaagccgccgccagtgccatttgttccgtggtaaggagaaaattatcactcgacgtgtccgcagcattttataacgctgctgcaaagtttgtcgcagcatggacattgaaggctgcagcgaaagcagcaaatgtatcaataccctggacccacaagggtgcagccgggctgtctaggtatgtggcgaggctaaacgccgccgcctcaacactgcctgagactactgtcgtgagacgcaaacaccctgccgcaatgccccacctgctgaaagcagccgcacgatggatgtgcctcagaagattcataataaacgcttctttctgtgggtcaccctacaaagccgcttacatggacgatgtggtcctcggagtgaatgccctctggttccatatcagctgcctgacattcaaggcagccgccacccccgctcgtgtgacaggaggtgtcttcaaagccgcggcactgactttcggtcgggaaactgtattggaatataagcaggccttcacattctccccaacatacaagtga

SEQ ID NO: 398 - protein, 344 aa, Artificial sequence
  Amino acid sequence of HBV-HTL epitope
MGTSFVYVPSALNPADGPGPGLCQVFADATPTGWGLGPGPGRHYLHTLWKAGILYKGPGPGPHHTALRQAILCWGELMTLAGPGPGESRLVVDFSQFSRGNGPGPGPFLLAQFTSAICSVVGPGPGLVPFVQWFVGLSPTVGPGPGLHLYSHPIILGFRKIGPGPGSSNLSWLSLDVSAAFGPGPGLQSLTNLLSSNLSWLGPGPGAGFFLLTRILTIPQSGPGPGVSFGVWIRTPPAYRPPNAPIGPGPGVGPLTVNEKRRLKLIGPGPGKQCFRKLPVNRPIDWGPGPGAANWILRGTSFVYVPGPGPGKQAFTFSPTYKAFLCGPGPGAKFVAAWTLKAAA

SEQ ID NO: 399 - DNA, 1035 bp, Artificial sequence
  Nucleotide sequence encoding amino acid sequence of HBV-HTL epitope
atgggaacttcttttgtgtatgtcccttccgctctgaacccagcagacggacccgggcctggcctgtgccaggtcttcgccgacgcaactcccacagggtgggggctggggccaggaccaggcaggcactacctgcatactctgtggaaggcaggaatcctctataaagggcccggcccaggccctcaccacaccgccctgaggcaggccatcctgtgctggggggagctcatgaccctggccggacctggacccggggagagcagactggtggtggatttcagccaattcagcagaggaaacggacccggccctgggccttttctgctggctcagtttacatctgctatttgttctgtggtcggccccgggcccggactcgtgcctttcgtgcagtggttcgtgggactgtcccctacagtcgggcccggcccagggctgcatctgtactcccacccaatcatcctcggcttccgcaagattggacccggcccaggctccagcaatctctcctggctctctctggacgtgtctgccgcctttggccctggaccaggcctgcaaagcctgactaatctgctcagcagcaacctgtcctggctgggacctggcccaggggctggcttctttctgctcacccggattctcacaattccccagtccggaccaggaccaggagtcagtttcggggtgtggatcaggacccctcctgcttatagaccacccaatgctccaatcggccccggccctggcgtcgggccactgaccgtgaatgagaagcgccggctgaagctgatcggccctggccctggcaagcagtgctttcgcaaactgcccgtgaacagacctattgattggggccccggccctggagcagccaactggattctcaggggaacaagcttcgtctacgtgcccgggcccggaccagggaagcaggcttttaccttctctcccacttacaaggccttcctctgtgggccaggccccggcgccaagtttgtggcagcatggaccctcaaagccgctgcctga

SEQ ID NO: 400 - protein, 16 aa, Homo sapiens
MGTSFVYVPSALNPAD

SEQ ID NO: 401 - protein, 15 aa, Homo sapiens
LCQVFADATPTGWGL

SEQ ID NO: 402 - protein, 15 aa, Homo sapiens
RHYLHTLWKAGILYK

SEQ ID NO: 403 - protein, 20 aa, Homo sapiens
PHHTALRQAILCWGELMTLA

SEQ ID NO: 404 - protein, 15 aa, Homo sapiens
ESRLVVDFSQFSRGN

SEQ ID NO: 405 - protein, 15 aa, Homo sapiens
PFLLAQFTSAICSVV

SEQ ID NO: 406 - protein, 15 aa, Homo sapiens
LVPFVQWFVGLSPTV

SEQ ID NO: 407 - protein, 15 aa, Homo sapiens
LHLYSHPIILGFRKI

SEQ ID NO: 408 - protein, 15 aa, Homo sapiens
SSNLSWLSLDVSAAF

SEQ ID NO: 409 - protein, 15 aa, Homo sapiens
LQSLTNLLSSNLSWL

SEQ ID NO: 410 - protein, 15 aa, Homo sapiens
AGFFLLTRILTIPQS

SEQ ID NO: 411 - protein, 20 aa, Homo sapiens
VSFGVWIRTPPAYRPPNAPI

SEQ ID NO: 412 - protein, 15 aa, Homo sapiens
VGPLTVNEKRRLKLI

SEQ ID NO: 413 - protein, 15 aa, Homo sapiens
KQCFRKLPVNRPIDW

SEQ ID NO: 414 - protein, 15 aa, Homo sapiens
AANWILRGTSFVYVP

SEQ ID NO: 415 - protein, 15 aa, Homo sapiens
KQAFTFSPTYKAFLC

SEQ ID NO: 416 - protein, 5 aa, Unknown
  Artificial peptide linker
GPGPG

SEQ ID NO: 417 - protein, 4 aa, Unknown
  Artificial peptide linker
GAAA

SEQ ID NO: 418 - protein, 4 aa, Unknown
  Artificial peptide linker
NAAA

SEQ ID NO: 419 - protein, 4 aa, Unknown
  Artificial peptide linker
KAAA

(19) United States
(12) Patent Application Publication
(10) Pub. No.: US 2004/0248113 A1
(43) Pub. Date: Dec. 9, 2004

(54) Method and system for optimizing multi-epitope nucleic acid constructs and peptides encoded thereby

(76) Inventors: Alessandro Sette, La Jolla, CA (US); Robert Chesnut, Cardiff-by-the-Sea, CA (US); Brian D Livingston, San Diego, CA (US); Denise Marie Baker, Poway, CA (US); Mark J Newman, Carlsbad, CA (US); David H Brown, San Diego, CA (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/474,960

(22) PCT Filed: Mar. 28, 2002
(86) PCT No.: PCT/US02/09877

Related U.S. Application Data
(63) Continuation-in-part of application No. 09/894,018, filed on Jun. 27, 2001.
Application No. 09/894,018 is a continuation-in-part of application No. PCT/US00/35568, filed on Dec. 28, 2000.
(60) Provisional application No. 60/284,221, filed on Apr. 16, 2001.
(60) Provisional application No. 60/173,390, filed on Dec. 28, 1999.

(51) Int. Cl. C12Q 1/68; G06F 19/00; G01N 33/48; G01N 33/50

(52) U.S. Cl. 435/6; 702/20

(57) ABSTRACT

The invention relates to the field of biology. In particular, the invention relates to a method and system for designing optimized multi-epitope vaccines having selected combinations of spacer nucleic acid at the junctions of the multi-epitope constructs encoding a plurality of CTL and/or HTL eoutioe oeotudes so as to minimize the number of junciontal epitopes and provide vaccines with increased immunogenicity.

### BACKGROUND

This present invention relates to the field of biology. In particular, it relates to multi-epitope nucleic acid vaccines and methods of designing such vaccines to provide increased immunogenicity.

The technology relevant to multi-epitope (“minigene” or “EpiGene™”) vaccines is developing. Several independent studies have established that induction of simultaneous immune responses against multiple epitopes can be achieved. For example, responses against a large number of T cell specificities can be induced and detected. In natural situations, Doolan et al (Immunity, Vol. 7(1):97-112 (1997)) simultaneously detected recall T cell responses, against as many as 17 different P. falciparum epitopes using PBMC from a single donor. Similarly, Bertoni and colleagues (J Clin Invest, Vol. 100(3):503-13 (1997)) detected simultaneous CTL responses against 12 different HBV-derived epitopes in a single donor. In terms of immunization with multi-epitope nucleic acid vaccines, several examples have been reported where multiple T cell responses were induced. For example, minigene vaccines composed of approximately ten MHC Class I epitopes in which all epitopes were immunogenic and/or antigenic have been reported. Specifically, minigene vaccines composed of 9 EBV (Thomson et al., Proc Natl Acad Sci USA, Vol. 92(13):5845-9 (1995)), 7 HIV (Woodberry et al., J Virol, Vol. 73(7):5320-5 (1999)) 10 murine (Thomson et al., J Immunol, Vol. 160(4):1717-23 (1998)) and 10 tumor-derived (Mateo et al., J Immunol, Vol. 163(7):4058-63 (1999)) epitopes have been shown to be active. It has also been shown that a multi-epitope DNA plasmid encoding nine different HLA-A2.1- and A11-restricted epitopes derived from HBV and HIV induced CTL against all epitopes (Ishioka et al., J Immunol, Vol. 162(7):3915-25 (1999)).

Thus, minigene vaccines containing multiple MHC Class I and Class II (i.e., CTL) epitopes can be designed, and presentation and recognition can be obtained for all epitopes. However, the immunogenicity of multi-epitope constructs appears to be strongly influenced by a number of variables, a number of which have heretofore been unknown. For example, the immunogenicity (or antigenicity) of the same epitope expressed in the context of need to identify strategies to optimize multi-epitope vaccine constructs. Such optimization is important in terms of induction of potent immune responses and ultimately, for clinical efficacy. Accordingly, the present invention provides strategies to optimize antigenicity and immunogenicity of multi-epitope vaccines encompassing a large number of epitopes, and optimized multi-epitope vaccines, particularly minigene vaccines, generated in accordance with these strategies.

The following paragraphs provide a brief review of some of the main variables potentially influencing minigene immunogenicity, epitope processing, and presentation on antigen presenting cells (APCs) in association with Class I and Class II MHC molecules.

Immunodominance

Of the many thousand possible peptides that are encoded by a complex foreign pathogen, only a small fraction ends up in a peptide form capable of binding to MHC Class I antigens and thus of being recognized by T cells. This phenomenon, of obvious potential impact on the development of a multi-epitope vaccine, is known as immunodominance (Yewdell et al., Annu Rev Immunol, 17:51-88 (1999)). Several major variables contribute to immunodominance. Herein, we describe variables affecting the generation of the appropriate peptides, both in qualitative and quantitative terms, as a result of intracellular processing.

Junctional Epitopes

A junctional epitope is defined as an epitope created due to the juxtaposition of two other epitopes. The new epitope is composed of a C-terminal section derived from a first epitope, and an N-terminal section derived from a second epitope. Creation of junctional epitopes is a potential problem in the design of multi-epitope minigene vaccines, for both Class I and Class II restricted epitopes for the following reasons. Firstly, when developing a minigene composed of, or containing, human epitopes, which are typically tested for immunogenicity in HLA transgenic laboratory animals, the creation of murine epitopes could create undesired immunodominance effects. Secondly, the creation of new, unintended epitopes for human HLA Class I or Class II molecules could elicit in vaccine recipients, new T cell specificities that are not expressed by infected cells or tumors that are the targets-induced T cell responses. These responses are by definition irrelevant and ineffective and could even be counterproductive, by creating undesired immunodominance effects.

The existence of junctional epitopes has been documented in a variety of different experimental situations. Gefter and collaborators first demonstrated the effect in a system in which two different Class II restricted epitopes were juxtaposed and colinearly synthesized (Perkins et al., J Immunol, Vol. 146(7):2137-44 (1991)). The effect was so marked that the immune system recognition of the epitopes could be completely “silenced” by these new junctional epitopes (Wang et al., Cell Immunol, Vol. 143(2):284-97 (1992)). Helper T cells directed against junctional epitopes were also observed in humans as a result of immunization with a synthetic lipopeptide, which was composed of an HLA-A2-restricted HBV-derived immunodominant CTL epitope, and a universal Tetanus Toxoid-derived HTL epitope (Livingston et al, J Immunol, Vol. 159(3):1383-92 (1997)). Thus, the creation of junctional epitopes are a major consideration in the design of multi-epitope constructs.

The present invention provides methods of addressing this problem and avoiding or minimizing the occurrence of junctional epitopes.

Flanking Regions

Class I restricted epitopes are generated by a complex process (Yewdell et al., Annu Rev Immunol, 17:51-88 (1999)). Limited proteolysis involving endoproteases and potential trimming by exoproteases is followed by translocation across the endoplasmic reticulum (ER) membrane by transporters associated with antigen processing (TAP) molecules. The major cytosolic protease complex involved in generation of antigenic peptides, and their precursors, is the proteosome (Niedermann et al., Immunity, Vol. 2(3):289-99 (1995)), although ER trimming of CTL precursors has also been demonstrated (Paz et al., Immunity Vol. 11(2):241-51 (1999)). It has long been debated whether or not the residues immediately flanking the C and N terminus of the epitope, have an influence on the efficiency of epitope generation.

The yield and availability of processed epitope has been implicated as a major variable in determining immunogenicity and could thus clearly have a major impact on overall minigene potency in that the magnitude of immune response can be directly proportional to the amount of epitope bound by MHC and displayed for T cell recognition. Several studies have provided evidence that this is indeed the case. For example, induction of virus-specific CTL that is essentially proportional to epitope density (Wherry et al., J Immunol, Vol. 163(7):3735-45 (1999)) has been observed. Further, recombinant minigenes, which encode a preprocessed optimal epitope, have been used to induce higher levels of epitope expression than naturally observed with full-length protein (Anton et al., J Immunol, Vol. 158(6):2535-42 (1997)). In general, minigene priming has been shown to be more effective than priming with the whole antigen (Restifo et al., J Immunol, Vol. 154(9):4414-22 (1995); Ishioka et al., J Immunol, Vol. 162(7):3915-25 (1999)), even though some exceptions have been noted (Iwasaki et al., Vaccine, Vol. 17(15-16):2081-8 (1999)).

Early studies concluded that residues within the epitope (Hahn et al., J Exp Med, Vol. 176(5):1335-41 (1992)) primarily regulate immunogenicity. Similar conclusions were reached by other studies, mostly based on grafting an epitope in an unrelated gene, or in the same gene, but in a different location (Chimini et al., J Exp Med, Vol. 169(1):297-302 (1989); Hahn et al., J Exp Med, Vol. 174(3):733-6 (1991)). Other experiments however (Del Val et al., Cell, Vol. 66(6):1145-53 (1991); Hahn et al., J Exp Med, Vol. 176(5):1335-41 (1992)), suggested that residues localized directly adjacent to the CTL epitope can directly influence recognition (Couillin et al., J Exp Med, Vol. 180(3):1129-34 (1994); Bergmann et al., J Virol. Vol. 68(8):5306-10 (1994)). In the context of minigene vaccines, the controversy has been renewed. Shastri and coworkers (Shastri et al., J Immunol, Vol. 155(9):4339-46 (1995)) found that T cell responses were not significantly affected by varying the N-terminal flanking residue but were inhibited by the addition of a single C-terminal flanking residue. The most dramatic inhibition was observed with isoleucine, leucine, cysteine, and proline as the C-terminal flanking residues. In contrast, Gileadi (Gileadi et al., Eur J Immunol, Vol. 29(7):2213-22 (1999)) reported profound effects as a function of the residues located at the N terminus of mouse influenza virus epitopes. Bergmann and coworkers found that aromatic, basic and alanine residues supported efficient epitope recognition, while G and P residues were strongly inhibitory (Bergmann et al., J Immunol, Vol. 157(8):3242-9 (1996)). In contrast, Lippolis (Lippolis et al., J Virol, Vol. 69(5):3134-46 (1995)) concluded that substituting flanking residues did not effect recognition. However, only rather conservative substitutions which are unlikely to affect proteosome specificity, were tested.

It appears that the specificity of these effects, and in general of natural epitopes, roughly correlates with proteosome specificity. For example, proteosome specificity is partly trypsin-like (Niedermann et al., Immunity, Vol. 2(3):289-99 (1995)), with cleavage following basic amino acids. Nevertheless, efficient cleavage of the carboxyl side of hydrophobic and acidic residues is also possible. Consistent with these specificities are the studies of Sherman and collaborators, which found that an R to H mutation at the position following the C-terminus of a p53 epitope affects proteosome-mediated processing of the protein (Theobald et al., J Exp Med, Vol. 188(6):1017-28 (1998)). Several other studies (Hanke et al., J Gen Virol, Vol. 79 (Pt 1):83-90 (1998); Thomson et al., Proc Natl Acad Sci USA, Vol. 92(13):5845-9 (1995)) indicated that minigenes can be constructed utilizing minimal epitopes, and that these flanking sequences appear not be required, although the potential for further optimization by the use of flanking regions was also acknowledged.

In sum, for HLA Class I epitopes, the effects of flanking regions on processing and presentation of CTL epitopes is as yet undefined. A systematic analysis of the effect of modulation of flanking regions has not been performed for minigene vaccines. Thus, analysis utilizing minigene vaccines encoding epitopes restricted by human Class I in general is needed. The present invention provides such an analysis and accordingly, provides multi-epitope vaccine constructs optimized for immunogenicity and antigenicity, and methods of designing such constructs.

HLA Class II peptide complexes are also generated as a result of a complex series of events that is distinct from HLA Class I processing. The processing pathway involves association with Invariant chain (Ii), its transport to specialized compartnents, the degradation of Ii to CLIP, and HLA-DM catalyzed removal of CLIP (see (Blum et al., Crit Rev Immunol, Vol. 17(5-6):411-7 (1997); Arndt et al., Immunol Res, Vol. 16(3):261-72 (1997)) for review. Moreover, there is potentially crucial role of various cathepsins in general, and cathepsin S and L in particular, in Ii degradation (Nakagawa et al., Immunity, Vol. 10(2):207-17 (1999)). In terms of generation of functional epitopes however, the process appears to be somewhat less selective (Chapman H. A., Curr Opin Immunol, Vol. 10(1):93-102 (1998)), and peptides of many sizes can bind to MHC Class IMHC Class II (Hunt et al., Science, Vol. 256(5065):1817-20 (1992)). Most or all of the possible peptides appear to be generated (Moudgil et al., J Immunol, Vol. 159(6):2574-9 (1997); and Thomson et al., J Virol, Vol. 72(3):2246-52 (1998)). Thus, as compared to the issue of flanking regions, the creation of junctional epitopes can be a more serious concern in particular embodiments.

### SUMMARY

The invention provides a method and system for optimizing the efficacy of multi-epitope vaccines so as to minimize the number of junctional epitopes and maximize, or at least increase, the immunogenicity and/or antigenicity of multi-epitope vaccines. In particular, the present invention provides multi-epitope nucleic acid constructs encoding a plurality of CTL and/or HTL epitopes.

In one embodiment of the invention, a computerized method for designing a multi-epitope construct having multiple epitopes includes the steps of: storing a plurality of input parameters in a memory of a computer system, the input parameters including a plurality of epitopes, at least one motif for identifying junctional epitopes, a plurality of amino acid insertions and at least one enhancement weight value for each insertion; generating a list of epitope pairs from the plurality of epitopes; determining for each epitope pair at least one optimum combination of amino acid insertions based on the at least one motif, the plurality of insertions and the at least one enhancement weight value for each insertion; and identifying at least one optimum arrangement of the plurality of epitopes, wherein a respective one of the at least one optimum combination of amino acid insertions is inserted at a respective junction of two epitopes, so as to provide an optimized multi-epitope construct. In a preferred embodiment, the step of identifying at least one optimum arrangement of epitopes may be accomplished by performing either an exhaustive search wherein all permutations of arrangements of the plurality of epitopes are evaluated or a stochastic search wherein only a subset of all permutations of arrangements of the plurality of epitopes are evaluated.

In a further embodiment, the method determines for each epitope pair at least one optimum combination of amino acid insertions by calculating a function value (F) for each possible combination of insertions for each epitope pair, wherein the number of insertions in a combination may range from 0 to a maximum number of insertions (MaxInsertions) value input by a user, and the function value is calculated in accordance with the equation F=(C+N)/J, when J>0, and F=2(C+N), when J=0, wherein C equals the enhancement weight value of a C+1 flanking amino acid, N equals the enhancement weight value of an N−1 flanking amino acid, and J equals the number of junctional epitopes detected for each respective combination of insertions in an epitope pair based on said at least one motif.

In another embodiment of the invention, a computer system for designing a multi-epitope construct having multiple epitopes, includes: a memory for storing a plurality of input parameters such as a plurality of epitopes, at least one motif for identifying junctional epitopes, a plurality of amino acid insertions and at least one enhancement weight value for each insertion; a processor for retrieving the input parameters from memory and generating a list of epitope pairs from the plurality of epitopes; wherein the processor further determines for each epitope pair at least one optimum combination of amino acid insertions, based on the at least one motif, the plurality of insertions and the at least one enhancement weight value for each insertion. The processor further identifies at least one optimum arrangement of the plurality of epitopes, wherein a respective one of the optimum combinations of amino acid insertions are inserted at a respective junction of two epitopes, to provide an optimized multi-epitope construct; and a display monitor, coupled to the processor, for displaying at least one optimum arrangement of the plurality of epitopes to a user.

In a further embodiment, the invention provides a data storage device storing a computer program for designing a multi-epitope construct having multiple epitopes, the computer program, when executed by a computer system, performing a process that, includes the steps of: retrieving a plurality of input parameters from a memory of a computer system, the input parameters including, for example, a plurality of epitopes, at least one motif for identifying junctional epitopes, a plurality of amino acid insertions and at least one enhancement weight value for each insertion; generating a list of epitope pairs from the plurality of epitopes; determining for each epitope pair at least one optimum combination of amino acid insertions based on the at least one motif, the plurality of insertions and the at least one enhancement weight value for each insertion; and identifying at least one optimum arrangement of the plurality of epitopes, wherein a respective one of the at least one optimum combination of amino acid insertions is inserted at a respective junction of two epitopes, so as to provide an optimized multi-epitope construct.

In another embodiment, the invention provides a method and system for designing a multi-epitope construct that comprises multiple epitopes. The method comprising steps of: (i) sorting the multiple epitopes to minimize the number of junctional epitopes; (ii) introducing a flanking amino acid residue at a C+1 position of an epitope to be included within the multi-epitope construct; (iii) introducing one or more amino acid spacer residues between two epitopes of the multi-epitope construct, wherein the spacer prevents the occurrence of a junctional epitope; and, (iv) selecting one or more multi-epitope constructs that have a minimal number of junctional epitopes, a minimal number of amino acid spacer residues, and a maximum number of flanking amino acid residues at a C+1 position relative to each epitope. In some embodiments, the spacer residues are independently selected from residues that are not known HLA Class II primary anchor residues. In particular embodiments, introducing the spacer residues prevents the occurrence of an HTL epitope. Such a spacer often comprises at least 5 amino acid residues independently selected from the group consisting of G, P, and N. In some embodiments the spacer is GPGPG.

In some embodiments, introducing the spacer residues prevents the occurrence of a CTL epitope and further, wherein the spacer is 1, 2, 3, 4, 5, 6, 7, or 8 amino acid residues independently selected from the group consisting of A and G. Often, the flanking residue is introduced at the C+1 position of a CTL epitope and is selected from the group consisting of K, R, N, G, and A. In some embodiments, the flanking residue is adjacent to the spacer sequence. The method of the invention can also include substituting an N-terminal residue of an epitope that is adjacent to a C-terminus of an adjacent epitope within the multi-epitope construct with a residue selected from the group consisting of K, R, N, G, and A.

In some embodiments, the method of the invention can also comprise a step of predicting a structure of the multi-epitope construct, and further, selecting one or more constructs that have a maximal structure, i.e., that are processed by an HLA processing pathway to produce all of the epitopes comprised by the construct. In some embodiments, the multi-epitope construct encodes EP-HIV-1090 as set out in FIG. 9, HIV-CPT as set out in FIG. 9, or HIV-TC as set out in FIG. 9.

In another embodiment of the invention, a system for optimizing multi-epitope constructs include a computer system having a processor (e.g., central processing unit) and at least one memory coupled to the processor for storing instructions executed by the processor and data to be manipulated (i.e., processed) by the processor. The computer system further includes an input device (e.g., keyboard) coupled to the processor and the at least one memory for allowing a user to input desired parameters and information to be accessed by the processor. The processor may be a single CPU or a plurality of different processing devices/circuits integrated onto a single integrated circuit chip. Alternatively, the processor may be a collection of discrete processing devices/circuits selectively coupled to one another via either direct wire/conductor connections or via a data bus. Similarly, the at least one memory may be one large memory device (e.g., EPROM), or a collection of a plurality of discrete memory devices (e.g., EEPROM, EPROM, RAM, DRAM, SDRAM, Flash, etc.) selectively coupled to one another for selectively storing data and/or program information (i.e., instructions executed by the processor). Those of ordinary skill in the art would easily be able to implement a desired computer system architecture to perform the operations and functions disclosed herein.

In one embodiment, the computer system includes a display monitor for displaying information, instructions, images, graphics, etc. The computer system receives user inputs via a keyboard. These user input parameters may include, for example, the number of insertions (i.e., flanking residues and spacer residues), the peptides to be processed, the C+1 and N−1 weighting values for each amino acid, and the motifs to use for searching for junctional epitopes. Based on these input values/parameters, the computer system executes a “Junctional Analyzer” software program which automatically determines the number of junctional epitope for each peptide pair and also calculates an “enhancement” value for each combination of flanking residues and spacers that may be inserted at the junction of each peptide pair. The results of the junctional analyzer program are then used in either an exhaustive or stochastic search program which determines the “optimal” combination or linkage of the entire set of peptides to create a multi-epitope polypeptide, or nucleic acids, having a minimal number of junctional epitopes and a maximum functional (e.g., immunogenicity) value.

In one embodiment, if the number of peptides to be processed by the computer system is less than fourteen, an exhaustive search program is executed by the computer system which examines all permutations of the peptides making up the polypeptide to find the permutation with the “best” or “optimal” function value. In one embodiment, the function value is calculated using the equation (Ce+Ne)/J when J is greater than zero and 2*(Ce+Ne) when J is equal to zero, where Ce is the enhancement “weight” value of an amino acid at the C+1 position of a peptide, Ne is the enhancement “weight” value of an amino acid at the N−1 position of a peptide, and J is the number of junctional epitopes contained in the polypeptide encoded by multi-epitope nucleic acid sequence. Thus, maximizing this function value will identify the peptide pairs having the least number of junctional epitopes and the maximum enhancement weight value for flanking residues. If the number of peptides to be processed is fourteen or more, the computer system executes a stochastic search program that uses a “Monte Carlo” technique to examine many regions of the permutation space to find the best estimate of the optimum arrangement of peptides (e.g., having the maximum function value).

In a further embodiment, the computer system allows a user to input parameter values which format or limit the output results of the exhaustive or stochastic search program. For example, a user may input the maximum number of results having the same function value (“MaxDuplicateFunctionValue=X”) to limit the number of permutations that are generated as a result of the search. Since it is possible for the search programs to find many arrangements that give the same function value, it may be desirable to prevent the output file from being filled by a large number of equivalent solutions. Once this limit is reached no more results are reported until a larger or “better” function value is found. As another example, the user may input the maximum number of “hits” per probe during a stochastic search process. This parameter prevents the stochastic search program from generating too much output on a single probe. In a preferred embodiment, the number of permutations examined in a single probe is limited by several factors: the amount of time set for each probe in the input text file; the speed of the computer, and the values of the parameters “MaxHitsPerProbe” and “MaxDuplicateFunctionValues.” The algorithms used to generate and-select permutations for analysis may be in accordance with well-known recursive algorithms found in many computer science text books. For example, six permutations of three things taken three at a time would be generated in the following sequence: ABC; ACB; BAC; BCA; CBA; CAB. As a further example of an input parameter, a user may input how the stochastic search is performed, e.g., randomly, statistically or other methodology; the maximum time allowed for each probe (e.g., 5 minutes); and the number of probes to perform.

Also disclosed herein are multi-epitope constructs designed by the methods described above and hereafter. The multi-epitope constructs include spacer nucleic acids between a subset of the epitope nucleic acids or all of the epitope nucleic acids. One or more of the spacer nucleic acids may encode amino acid sequences different from amino acid sequences encoded by other spacer nucleic acids to optimize epitope processing and to minimize the presence of junctional epitopes.

### DEFINITIONS

The following definitions are provided to enable one of ordinary skill in the art to understand some of the preferred embodiments of invention disclosed herein. It is understood, however, that these definitions are exemplary only and should not be used to limit the scope of the invention as set forth in the claims. Those of ordinary skill in the art will be able to construct slight modifications to the definitions below and utilize such modified definitions to understand and practice the invention disclosed herein. Such modifications, which would be obvious to one of ordinary skill in the art, as they may be applicable to the claims set forth below, are considered to be within the scope of the present invention.

Throughout this disclosure, “binding data” results are often expressed in terms of “IC₅₀'s.” IC₅₀ is the concentration of peptide in a binding assay at which 50% inhibition of binding of a reference peptide is observed. Given the conditions in which the assays are run (i.e., limiting HLA proteins and labeled peptide concentrations), these values approximate K_{D} values. Assays for determining binding are described in detail, e.g., in PCT publications WO 94/20127 and WO 94/03205. It should be noted that IC₅₀ values can change, often dramatically, if the assay conditions are varied, and depending on the particular reagents used (e.g., HLA preparation, etc.). For example, excessive concentrations of HLA molecules will increase the apparent measured IC₅₀ of a given ligand. Alternatively, binding is expressed relative to a reference peptide. Although as a particular assay becomes more, or less, sensitive, the IC₅₀'s of the peptides tested may change somewhat, the binding relative to the reference peptide will not significantly change. For example, in an assay run under conditions such that the IC₅₀ of the reference peptide increases 10-fold, the IC₅₀ values of the test peptides will also shift approximately 10-fold. Therefore, to avoid ambiguities, the assessment of whether a peptide is a good, intermediate, weak, or negative binder is generally based on its IC₅₀, relative to the IC₅₀ of a standard peptide. Binding may also be determined using other assay systems including those using: live cells (e.g., Ceppellini et al., Nature 339:392, 1989; Christnick et al., Nature 352:67, 1991; Busch et al., Int. Immunol. 2:443, 19990; Hill et al., J. Immunol. 147:189, 1991; del Guercio et al., J. Immunol. 154:685, 1995), cell free systems using detergent lysates (e.g., Cerundolo et al., J. Immunol. 21:2069, 1991), immobilized purified MHC (e.g., Hill et al., J. Immunol. 152, 2890, 1994; Marshall et al., J. Immunol. 152:4946, 1994), ELISA systems (e.g., Reay et al., EMBO J. 11:2829, 1992), surface plasmon resonance (e.g., Khilko et al., J. Biol. Chem. 268:15425, 1993); high flux soluble phase assays (Hammer et al., J. Exp. Med. 180:2353, 1994), and measurement of class I MHC stabilization or assembly (e.g., Ljunggren et al., Nature 346:476, 1990; Schumacher et al., Cell 62:563, 1990; Townsend et al., Cell 62:285, 1990; Parker et al., J. Immunol. 149:1896, 1992).

The designation of a residue position in an epitope as the “carboxyl terminus” or the “carboxyl terminal position” refers to the residue position at the end of the epitope that is nearest to the carboxyl terminus of a peptide, which is designated using conventional nomenclature as defined below. “C+1” refers to the residue or position immediately following the C-terminal residue of the epitope, i.e., refers to the residue flanking the C-terminus of the epitope. The “carboxyl terminal position” of the epitope occurring at the carboxyl end of the multi-epitope construct may or may not actually correspond to the carboxyl terminal end of polypeptide. In preferred embodiments, the epitopes employed in the optimized multi-epitope constructs are motif-bearing epitopes and the carboxyl terminus of the epitope is defined with respect to primary anchor residues corresponding to a particular motif.

The designation of a residue position in an epitope as “amino terminus” or “amino-terminal position” refers to the residue position at the end of the epitope which is nearest to the amino terminus of a peptide, which is designated using conventional nomenclature as defined below. “N−1” refers to the residue or position immediately adjacent to the epitope at the amino terminal end (position number 1) of an eptiope. The “amino terminal position” of the epitope occurring at the amino terminal end of the multi-epitope construct may or may not actually corresponds to the amino terminal end of the polypeptide. In preferred embodiments, the epitopes employed in the optimized multi-epitope constructs are motif-bearing epitopes and the amino terminus of the epitope is defined with respect to primary anchor residues corresponding to a particular motif.

A “computer” or “computer system” generally includes: a processor; at least one information storage/retrieval apparatus such as, for example, a hard drive, a disk drive or a tape drive; at least one input apparatus such as, for example, a keyboard, a mouse, a touch screen, or a microphone; and display structure. Additionally, the computer may include a communication channel in communication with a network such that remote users may communicate with the computer via the network to perform multi-epitope construct optimization functions disclosed herein. Such a computer may include more or less than what is listed above. The network may be a local area network (LAN), wide area network (WAN) or a global network such as the world wide web (e.g., the internet).

A “construct” as used herein generally denotes a composition that does not occur in nature. A construct can be produced by synthetic technologies, e.g., recombinant DNA preparation and expression or chemical synthetic techniques for nucleic or amino acids. A construct can also be produced by the addition or affiliation of one material with another such that the result is not found in nature in that form. A “multi-epitope construct” can be used interchangeably with the term “minigene” or “multi-epitope nucleic acid vaccine,” and comprises multiple epitope nucleic acids that encode peptide epitopes of any length that can bind to a molecule functioning in the immune system, preferably a class I HLA and a T-cell receptor or a class II HLA and a T-cell receptor. All of the epitope nucleic acids in a multi-epitope construct can encode class I HLA epitopes or class II HLA epitopes. Class I HLA-encoding epitope nucleic acids are referred to as CTL epitope nucleic acids, and class II HLA-encoding epitope nucleic acids are referred to as HTL epitope nucleic acids. Some multi-epitope constructs can have a subset of the multi-epitope nucleic acids encoding class I HLA epitopes and another subset of the multi-epitope nucleic acids encoding class II HLA epitopes. The CTL epitope nucleic acids preferably encode an epitope peptide of about eight to about thirteen amino acids in length, more preferably about eight to about eleven amino acids in length, and most preferably about nine amino acids in length. The HTL epitope nucleic acids can encode an epitope peptide of about seven to about twenty three, preferably about seven to about seventeen, more preferably about eleven to about fifteen, and most preferably about thirteen amino acids in length. The multi-epitope constructs described herein preferably include five or more, ten or more, fifteen or more, twenty or more, or twenty-five or more epitope nucleic acids. All of the epitope nucleic acids in a multi-epitope construct may be from one organism (e.g., the nucleotide sequence of every epitope nucleic acid may be present in HIV strains), or the multi-epitope construct may include epitope nucleic acids present in two or more different organisms (e.g., some epitopes from HIV and some from HCV). The term “EpiGene™” is used herein to refer to certain multi-epitope constructs. As described hereafter, one or more epitope nucleic acids in the multi-epitope construct may be flanked by a spacer nucleic acid.

A “multi-epitope vaccine,” which is synonyous with a “polyepitopic vaccine,” is a vaccine comprising multiple epitopes.

“Cross-reactive binding” indicates that a peptide is bound by more than one HLA molecule; a synonym is “degenerate binding.”

A “cryptic epitope” elicits a response by immunization with an isolated peptide, but the response is not cross-reactive in vitro when intact whole protein that comprises the epitope is used as an antigen.

A “dominant epitope” is an epitope that induces an immune response upon immunization with a whole native antigen (see, e.g., Sercarz, et al., Annu. Rev. Immunol. 11:729-766, 1993). Such a response is cross-reactive in vitro with an isolated peptide epitope.

With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) receptors. In an immune system setting, in vitro or in vivo, an epitope is the collective features of a molecule, such as primary, secondary and tertiary peptide structure, and charge, that together form a site recognized by an immunoglobulin, T cell receptor or HLA molecule. Throughout this disclosure epitope and peptide are often used interchangeably. It is to be appreciated, however, that isolated or purified protein or peptide molecules larger than and comprising an epitope of the invention are still within the bounds of the invention.

A “flanking residue” is a residue that is positioned next to an epitope. A flanking residue can be introduced or inserted at a position adjacent to the N-terminus or the C-terminus of an epitope.

An “immunogenic peptide” or “peptide epitope” is a peptide that comprises an allele-specific motif or supermotif such that the peptide will bind an HLA molecule and induce a CTL and/or HTL response. Thus, immunogenic peptides of the invention are capable of binding to an appropriate HLA molecule and thereafter inducing a cytotoxic T cell response, or a helper T cell response, to the antigen from which the immunogenic peptide is derived.

“Heteroclitic analogs” are defined herein as a peptide with increased potency for a specific T cell, as measured by increased responses to a given dose, or by a requirement of lesser amounts to achieve the same response. Advantages of heteroclitic analogs include that the epitopes can be more potent, or more economical (since a lower amount is required to achieve the same effect). In addition, modified epitopes might overcome antigen-specific T cell unresponsiveness (T cell tolerance).

“Human Leukocyte Antigen” or “HLA” is a human class I or class II Major Histocompatibility Complex (MHC) protein (see, e.g., Stites, et al., IMMUNOLOGY, 8^{TH} ED., Lange Publishing, Los Altos, Calif. (1994)).

An “HLA supertype or HLA family,” as used herein, describes sets of HLA molecules grouped based on shared peptide-binding specificities. HLA class I molecules that share similar binding affinity for peptides bearing certain amino acid motifs are grouped into such HLA supertypes. The terms HLA superfamily, HLA supertype family, HLA family, and HLA xx-like molecules (where xx denotes a particular HLA type), are synonyms.

As used herein, “high affinity” with respect to HLA class I molecules is defined as binding with an IC₅₀, or K_{D} value, of 50 nM or less; “intermediate affinity” with respect to HLA class I molecules is defined as binding with an IC₅₀ or K_{D} value of between about 50 and about 500 nM. “High affinity” with respect to binding to HLA class II molecules is defined as binding with an IC₅₀ or K_{D} value of 100 nM or less; “intermediate affinity” with respect to binding to HLA class II molecules is defined as binding with an IC₅₀ or K_{D} value of between about 100 and about 1000 nM.

An “IC₅₀” is the concentration of peptide in a binding assay at which 50% inhibition of binding of a reference peptide is observed. Depending on the conditions in which the assays are run (i.e., limiting HLA proteins and labeled peptide concentrations), these values may approximate K_{D} values.

The terms “identical” or percent “identity,” in the context of two or more peptide sequences, refer to two or more sequences or subsequences that are the same or have a specified percentage of amino acid residues that are the same, when compared and aligned for maximum correspondence over a comparison window, as measured using a sequence comparison algorithm or by manual alignment and visual inspection.

“Introducing” an amino acid residue at a particular position in a multi-epitope construct, e.g., adjacent, at the C-terminal side, to the C-terminus of the epitope, encompasses configuring multiple epitopes such that a desired residue is at a particular position, e.g., adjacent to the epitope, or such that a deleterious residue is not adjacent to the C-terminus of the epitope. The term also includes inserting an amino acid residue, preferably a preferred or intermediate amino acid residue, at a particular position. An amino acid residue can also be introduced into a sequence by substituting one amino acid residue for another. Preferably, such a substitution is made in accordance with analoging principles set forth, e.g., in co-pending U.S. Ser. No. 09/260,714 filed Mar. 1, 1999 and PCT application number PCT/US00/19774.

The phrases “isolated” or “biologically pure” refer to material that is substantially or essentially free from components which normally accompany the material as it is found in its native state. Thus, isolated peptides in accordance with the invention preferably do not contain materials normally associated with the peptides in their in situ environment.

“Link” or “join” refers to any method known in the art for functionally connecting peptides, including, without limitation, recombinant fusion, covalent bonding, disulfide bonding, ionic bonding, hydrogen bonding, and electrostatic bonding.

“Major Histocompatibility Complex” or “MHC” is a cluster of genes that plays a role in control of the cellular interactions responsible for physiologic immune responses. In humans, the MHC complex is also known as the HLA complex. For a detailed description of the MHC and HLA complexes, see, Paul, FUNDAMENTAL IMMUNOLOGY, 3^{RD} ED., Raven Press, New York, 1993.

As used herein, “middle of the peptide” is a position in a peptide that is neither an amino or a carboxyl terminus.

A “minimal number of junctional epitopes” as used herein refers to a number of junctional epitopes that is lower than what would be created using a random selection criteria.

The term “motif” refers to the pattern of residues in a peptide of defined length, usually a peptide of from about 8 to about 13 amino acids for a class I HLA motif and from about 6 to about 25 amino acids for a class II HLA motif, which is recognized by a particular HLA molecule. Peptide motifs are typically different for each protein encoded by each human HLA allele and differ in the pattern of the primary and secondary anchor residues.

A “negative binding residue” or “deleterious residue” is an amino acid which, if present at certain positions (typically not primary anchor positions) in a peptide epitope, results in decreased binding affinity of the peptide for the peptide's corresponding HLA molecule.

“Optimizing” refers to increasing the immunogenicity or antigenicity of a multi-epitope construct having at least one epitope pair by sorting epitopes to minimize the occurrence of junctional epitopes, inserting flanking residues that flank the C-terminus or N-terminus of an epitope, and inserting spacer residue to further prevent the occurrence of junctional epitopes or to provide a flanking residue. An increase in immunogenicity or antigenicity of an optimized multi-epitope construct is measured relative to a multi-epitope construct that has not been constructed based on the optimization parameters and is using assays known to those of skill in the art, e.g., assessment of immunogenicity in HLA transgenic mice, ELISPOT, inteferon-gamma release assays, tetramer staining, chromiun release assays, and presentation on dendritic cells.

The term “peptide” is used interchangeably with “oligopeptide” in the present specification to designate a series of residues, typically L-amino acids, connected one to the other, typically by peptide bonds between the α-amino and carboxyl groups of adjacent amino acids. The preferred CTL-inducing peptides of the invention are 13 residues or less in length and usually consist of between about 8 and about 11 residues, preferably 9 or 10 residues. The preferred HTL-inducing oligopeptides are less than about 50 residues in length and usually consist of between about 6 and about 30 residues, more usually between about 12 and 25, and often between about 15 and 20 residues.

A “PanDR binding peptide or PADRE® peptide” is a member of a family of molecules that binds more than one HLA class II DR molecule. The pattern that defines the PADRE® family of molecules can be thought of as an HLA Class II supermotif. PADRE® binds to most HLA-DR molecules and stimulates in vitro and in vivo human helper T lymphocyte (HTL) responses.

“Pharmaceutically acceptable” refers to a generally non-toxic, inert, and/or physiologically compatible composition.

“Presented to an HLA Class I processing pathway” means that the multi-epitope constructs are introduced into a cell such that they are largely processed by an HLA Class I processing pathway. Typically, multi-epitope constructs are introduced into the cells using expression vectors that encode the multi-epitope constructs. HLA Class II epitopes that are encoded by such a multi-epitope construct are also presented on Class II molecules, although the mechanism of entry of the epitopes into the Class II processing pathway is not defined.

A “primary anchor residue” or a “primary MHC anchor” is an amino acid at a specific position along a peptide sequence that is understood to provide a contact point between the immunogenic peptide and the HLA molecule. One to three, usually two, primary anchor residues within a peptide of defined length generally defines a “motif” for an immunogenic peptide. These residues are understood to fit in close contact with peptide binding grooves of an HLA molecule, with their side chains buried in specific pockets of the binding grooves themselves. In one embodiment, for example, the primary anchor residues of an HLA class I epitope are located at position 2 (from the amino terminal position) and at the carboxyl terminal position of a 9-residue peptide epitope in accordance with the invention. The primary anchor positions for each motif and supermotif are described, for example, in Tables I and III of PCT/US00/27766, or PCT/US00/19774. Preferred amino acids that can serve as in the anchors for most Class II epitopes consist of M and F in position one and V, M, S, T, A and C in position six. Tolerated amino acids that can occupy these positions for most Class II epitopes consist of L, I, V, W, and Y in position one and P, L and I in position six. The presence of these amino acids in positions one and six in Class II epitopes defines the HLA-DR1, 4, 7 supermotif The HLA-DR3 binding motif is defined by preferred amino acids from the group of L, I, V, M, F, Y and A in position one and D, E, N, Q, S and T in position four and K, R and H in position six. Other amino acids may be tolerated in these positions but they are not preferred.

Furthermore, analog peptides can be created by altering the presence or absence of particular residues in these primary anchor positions. Such analogs are used to modulate the binding affinity of a peptide comprising a particular motif or supermotif

“Promiscuous recognition” occurs where a distinct peptide is recognized by the same T cell clone in the context of various HLA molecules. Promiscuous recognition or binding is synonymous with cross-reactive binding.

A “protective immune response” or “therapeutic immune response” refers to a CTL and/or an HTL response to an antigen derived from an infectious agent or a tumor antigen, which in some way prevents or at least partially arrests disease symptoms, side effects or progression. The immune response may also include an antibody response that has been facilitated by the stimulation of helper T cells.

The term “residue” refers to an amino acid or amino acid mimetic incorporated into a peptide or protein by an amide bond or amide bond mimetic.

A “secondary anchor residue” is an amino acid at a position other than a primary anchor position in a peptide that may influence peptide binding. A secondary anchor residue occurs at a significantly higher frequency amongst bound peptides than would be expected by random distribution of amino acids at one position. The secondary anchor residues are said to occur at “secondary anchor positions.” A secondary anchor residue can be identified as a residue which is present at a higher frequency among high or intermediate affinity binding peptides, or a residue otherwise associated with high or intermediate affinity binding. For example, analog peptides can be created by altering the presence or absence of particular residues in these secondary anchor positions. Such analogs are used to finely modulate the binding affinity of a peptide comprising a particular motif or supermotif. The terminology “fixed peptide” is sometimes used to refer to an analog peptide.

“Sorting epitopes” refers to determining or designing an order of the epitopes in a multi-epitope construct.

A “spacer” refers to a sequence that is inserted between two epitopes in a multi-epitope construct to prevent the occurrence of junctional epitopes and/or to increase the efficiency of processing. A multi-epitope construct may have one or more spacer nucleic acids. A spacer nucleic acid may flank each epitope nucleic acid in a construct, or the spacer nucleic acid to epitope nucleic acid ratio may be about 2 to 10, about 5 to 10, about 6 to 10, about 7 to 10, about 8 to 10, or about 9 to 10, where a ratio of about 8 to 10 has been determined to yield favorable results for some constructs.

The spacer nucleic acid may encode one or more amino acids. A spacer nucleic acid flanking a class I HLA epitope in a multi-epitope construct is preferably between one and about eight amino acids in length. A spacer nucleic acid flanking a class II HLA epitope in a multi-epitope construct is preferably greater than five, six, seven, or more amino acids in length, and more preferably five or six amino acids in length.

The number of spacers in a construct, the number of amino acids in a spacer, and the amino acid composition of a spacer can be selected to optimize epitope processing and/or minimize junctional epitopes. It is preferred that spacers are selected by concomitantly optimizing epitope processing and junctional motifs. Suitable amino acids for optimizing epitope processing are described herein. Also, suitable amino acid spacing for minimizing the number of junctional epitopes in a construct are described herein for class I and class II HLAs. For example, spacers flanking class II HLA epitopes preferably include G, P, and/or N residues as these are not generally known to be primary anchor residues (see, e.g., PCT/US00/19774). A particularly preferred spacer for flanking a class II HLA epitope includes alternating G and P residues, for example, (GP)ₙ, (PG)ₙ, (GP)ₙG, (PG)ₙP, and so forth, where n is an integer between one and ten, preferably two or about two, and where a specific example of such a spacer is GPGPG. A preferred spacer, particularly for class I HLA epitopes, comprises one, two, three or more consecutive alanine (A) residues (see, for example, FIG. 23A, which depicts a spacer having three consecutive alanine residues).

In some multi-epitope constructs, it is sufficient that each spacer nucleic acid encodes the same amino acid sequence. In multi-epitope constructs having two spacer nucleic acids encoding the same amino acid sequence, the spacer nucleic acids encoding those spacers may have the same or different nucleotide sequences, where different nucleotide sequences may be preferred to decrease the likelihood of unintended recombination events when the multi-epitope construct is inserted into cells.

In other multi-epitope constructs, one or more of the spacer nucleic acids may encode different amino acid sequences. While many of the spacer nucleic acids may encode the same amino acid sequence in a multi-epitope construct, one, two, three, four, five or more spacer nucleic acids may encode different amino acid sequences, and it is possible that all of the spacer nucleic acids in a multi-epitope construct encode different amino acid sequences. Spacer nucleic acids may be optimized with respect to the epitope nucleic acids they flank by determining whether a spacer sequence will maximize epitope processing and/or minimize junctional epitopes, as described herein.

Multi-epitope constructs may be distinguished from one another according to whether the spacers in one construct optimize epitope processing or minimize junctional epitopes over another construct, and preferably, constructs may be distinguished where one construct is concomitantly optimized for epitope processing and junctional epitopes over the other. Computer assisted methods and in vitro and in vivo laboratory methods for determining whether a construct is optimized for epitope processing and junctional motifs are described herein.

A “subdominant epitope” is an epitope which evokes little or no response upon immunization with whole antigens which comprise the epitope, but for which a response can be obtained by immunization with an isolated epitope, and this response (unlike the case of cryptic epitopes) is detected when whole protein is used to recall the response in vitro or in vivo.

A “supermotif” is an amino acid sequence for a peptide that provides binding specificity shared by HLA molecules encoded by two or more HLA alleles. Preferably, a supermotif-bearing peptide is recognized with high or intermediate affinity (as defined herein) by two or more HLA antigens.

“Synthetic peptide” refers to a peptide that is not naturally occurring, but is man-made using such methods as chemical synthesis or recombinant DNA technology.

A “TCR contact residue” or “T cell receptor contact residue” is an amino acid residue in an epitope that is understood to be bound by a T cell receptor; these are defined herein as not being any primary MHC anchor. T cell receptor contact residues are defined as the position/positions in the peptide where all analogs tested induce T-cell recognition relative to that induced with a wildtype peptide.

The term “homology,” as used herein, refers to a degree of complementarity between two nucleotide sequences. The word “identity” may substitute for the word “homology” when a nucleic acid has the same nucleotide sequence as another nucleic acid. Sequence homology and sequence identity can also be determined by hybridization studies under high stringency and/or low stringency, and disclosed herein are nucleic acids that hybridize to the multi-epitope constructs under low stringency or under high stringency. Also, sequence homology and sequence identity can be determined by analyzing sequences using algorithms and computer programs known in the art. Such methods be used to assess whether a nucleic acid is identical or homologous to the multi-epitope constructs disclosed herein. The invention pertains in part to nucleotide sequences having 80% or more, 85% or more, 90% or more, 95% or more, 97% or more, 98% or more, or 99% or more identity to the nucleotide sequence of a multi-epitope construct disclosed herein.

As used herein, the term “stringent conditions” refers to conditions which permit hybridization between nucleotide sequences and the nucleotide sequences of the disclosed multi-epitope constructs. Suitable stringent conditions can be defined by, for example, the concentrations of salt or formamide in the prehybridization and hybridization solutions, or by the hybridization temperature, and are well known in the art. In particular, stringency can be increased by reducing the concentration of salt, increasing the concentration of formamide, or raising the hybridization temperature. For example, hybridization under high stringency conditions could occur in about 50% formamide at about 37° C. to 42° C. In particular, hybridization could occur under high stringency conditions at 42° C. in 50% formamide, 5×SSPE, 0.3% SDS, and 200 μg/ml sheared and denatured salmon sperm DNA or at 42° C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C. Hybridization could occur under reduced stringency conditions in about 35% to 25% formamide at about 30° C. to 35° C. For example, reduced stringency conditions could occur at 35° C. in 35% formamide, 5×SSPE, 0.3% SDS, and 200 μg/ml sheared and denatured salmon sperm DNA. The temperature range corresponding to a particular level of stringency can be further narrowed by calculating the purine to pyrimidine ratio of the nucleic acid of interest and adjusting the temperature accordingly. Variations on the above ranges and conditions are well known in the art.

In addition to utilizing hybridization studies to assess sequence identity or sequence homology, known computer programs may be used to determine whether a particular nucleic acid is homologous to a multi-epitope construct disclosed herein. An example of such a program is the Bestfit program (Wisconsin Sequence Analysis Package, Version 8 for Unix, Genetics Computer Group, University Research Park, 575 Science Drive, Madison, Wis. 53711), and other sequence alignment programs are known in the art and may be utilized for determining whether two or more nucleotide sequences are homologous. Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence, the parameters may be set such that the percentage of identity is calculated over the full length of the reference nucleotide sequence and that gaps in homology of up to 5% of the total number of nucleotides in the reference sequence are allowed.

The nomenclature used to describe peptide compounds follows the conventional practice wherein the amino group is presented to the left (the N-terminus) and the carboxyl group to the right (the C-terminus) of each amino acid residue. When amino acid residue positions are referred to in an epitope, they are numbered in an amino to carboxyl direction with position one being the position closest to the amino terminal end of the epitope, or the peptide or protein of which it may be a part. In the formulae representing selected specific embodiments of the present invention, the amino- and carboxyl-terminal groups, although not specifically shown, are in the form they would assume at physiologic pH values, unless otherwise specified. In the amino acid structure formulae, each residue is generally represented by standard three-letter or single-letter designations. The L-form of an amino acid residue is represented by a capital single letter or a capital first letter of a three-letter symbol, and the D-form for those amino acids having D-forms is represented by a lower case single letter or a lower case three letter symbol. Glycine has no asymmetric carbon atom and is simply referred to as “Gly” or G. Symbols for the amino acids are shown below.

| Single Letter | Three Letter | |
|---|---|---|
| Symbol | Symbol | Amino Acids |
| A | Ala | Alanine |
| C | Cys | Cysteine |
| D | Asp | Aspartic Acid |
| E | Glu | Glutamic Acid |
| F | Phe | Phenylalanine |
| G | Gly | Glycine |
| H | His | Histidine |
| I | Ile | Isoleucine |
| K | Lys | Lysine |
| L | Leu | Leucine |
| M | Met | Methionine |
| N | Asn | Asparagine |
| P | Pro | Proline |
| Q | Gln | Glutamine |
| R | Arg | Arginine |
| S | Ser | Serine |
| T | Thr | Threonine |
| V | Val | Valine |
| W | Trp | Tryptophan |
| Y | Tyr | Tyrosine |

Amino acid “chemical characteristics” are defined as: Aromatic (F, W, Y); Aliphatic-hydrophobic (L, I, V, M); Small polar (S, T, C); Large polar (Q, N); Acidic (D, E); Basic (R, H, K); Proline; Alanine; and Glycine.

Acronyms used herein are as follows:

APC: Antigen presenting cell

CD3: Pan T cell marker

CD4: Helper T lymphocyte marker

CD8: Cytotoxic T lymphocyte marker

CEA: Carcinoembryonic antigen

CFA: Complete Freund's Adjuvant

CTL: Cytotoxic T lymphocytes

DC: Dendritic cells. DC functioned as potent antigen presenting cells by stimulating cytokine release from CTL lines that were specific for a model peptide derived from hepatitis B virus (HBV). In vitro experiments using DC pulsed ex vivo with an HBV peptide epitope have stimulated CTL immune responses in vitro following delivery to naive mice.

DMSO: Dimethylsulfoxide

ELISA: Enzyme-linked immunosorbant assay

E:T: Effector:target ratio

FCS: Fetal calf serum

G-CSF: Granulocyte colony-stimulating factor

GM-CSF: Granulocyte-macrophage (monocyte)-colony stimulating factor

HBV: Hepatitis B virus

HER2/Neu: c-erbB-2

HLA: Human leukocyte antigen

HLA-DR: Human leukocyte antigen class II

HPLC: High Performance Liquid Chromatography

HTC: Helper T cells

HTL: Helper T Lymphocyte

ID: Identity

IFA: Incomplete Freund's Adjuvant

IFNγ: Interferon gamma

IL-4: Interleukin-4 cytokine

IV: Intravenous

LU_{30%}: Cytotoxic activity required to achieve 30% lysis at a 100:1 (E:T) ratio

MAb: Monoclonal antibody

MAGE: Melanoma antigen

MLR: Mixed lymphocyte reaction

MNC: Mononuclear cells

PB: Peripheral blood

PBMC: Peripheral blood mononuclear cell

SC: Subcutaneous

S.E.M.: Standard error of the mean

QD: Once a day dosing

TAA: Tumor associated antigen

TCR: T cell receptor

TNF: Tumor necrosis factor

WBC: White blood cells

This application may be relevant to U.S. Ser. No. 09/189,702 filed Nov. 10, 1998, which is a CIP of U.S. Ser. No. 08/205,713 filed Mar. 4, 1994, which is a CIP of Ser. No. 08/159,184 filed Nov. 29, 1993 and now abandoned, which is a CIP of Ser. No. 08/073,205 filed Jun. 4. 1993 and now abandoned, which is a CIP of Ser. No. 08/027,146 filed Mar. 5, 1993 and now abandoned. The present application is also related to U.S. Ser. No. 09/226,775, which is a CIP of U.S. Ser. No. 08/815,396, which claims the benefit of U.S. Ser. No. 60/013,113, now abandoned. Furthermore, the present application is related to U.S. Ser. No. 09/017,735, which is a CIP of abandoned U.S. Ser. No. 08/589,108; U.S. Ser. No. 08/753,622, U.S. Ser. No. 08/822,382, abandoned U.S. Ser. No. 60/013,980, U.S. Ser. No. 08/454,033, U.S. Ser. No. 09/116,424, and U.S. Ser. No. 08/349,177. The present application is also related to U.S. Ser. No. 09/017,524, U.S. Ser. No. 08/821,739, abandoned U.S. Ser. No. 60/013,833, U.S. Ser. No. 08/758,409, U.S. Ser. No. 08/589,107, U.S. Ser. No. 08/451,913, U.S. Ser. No. 08/186,266, U.S. Ser. No. 09/116,061, and U.S. Ser. No. 08/347,610, which is a CIP of U.S. Ser. No. 08/159,339, which is a CIP of abandoned U.S. Ser. No. 08/103,396, which is a CIP of abandoned U.S. Ser. No. 08/027,746which is a CIP of abandoned U.S. Ser. No. 07/926,666. The present application may also be relevant to U.S. Ser. No. 09/017,743, U.S. Ser. No. 08/753,615; U.S. Ser. No. 08/590,298, U.S. Ser. No. 09/115,400, and U.S. Ser. No. 08/452,843, which is a CIP of U.S. Ser. No. 08/344,824, which is a CIP of abandoned U.S. Ser. No. 08/278,634. The present application may also be related to provisional U.S. Ser. No. 60/087,192 and U.S. Ser. No. 09/009,953, which is a CIP of abandoned U.S. Ser. No. 60/036,713 and abandoned U.S. Ser. No. 60/037,432. In addition, the present application may be relevant to U.S. Ser. No. 09/098,584, and U.S. Ser. No. 09/239,043. The present application may also be relevant to co-pending U.S. Ser. No. 09/583,200 filed May 30, 2000, U.S. Ser. No. 09/260,714 filed Mar. 1, 1999, and U.S. Provisional Application “Heteroclitic Analogs And Related Methods,” Attorney Docket Number 018623-015810US filed Oct. 6, 2000. All of the above applications are incorporated herein by reference.

### BRIEF DESCRIPTION OF THE DRAWINGS

FIG. 1 illustrates data on three different multi-epitope constructs, incorporating 20 to 25 different CTL epitopes each.

FIG. 2 illustrates two different synthetic polypeptides (FIG. 2a) where the first construct incorporates four different epitopes linearly cosynthetized, and the second construct incorporates a GPGPG spacer. FIG. 2b illustrates the capacity of 2 nanomoles of these different constructs to prime for proliferative responses to the various epitopes in IA^{b} positive mice, compared to the responses induced by equimolar amounts of a pool of the same peptides (3 micrograms of each peptide).

FIG. 3 depicts the structure of multi-epitope DNA constructs. The HLA restriction is shown above each epitope, the A*0201 epitopes are bolded. The HLA binding affinity (IC₅₀ nM) is provided below each epitope. (a) Schematic of HIV-FT illustrating order of the encoded epitopes. (b) Schematics of the of the HBV-specific constructs. The C+1 amino acid relative to Core 18 is indicated with an arrow. The HBV-specific constructs with single amino acid insertions at the C₁ position of Core 18 are illustrated as HBV.1X.

FIG. 4 illustrates the immunogenicity of the HLA-A*0201 epitopes in HIV-FT in HLA-A*0201/K^{b} transgenic mice. (a) Representative CTL responses against epitopes Pol 498 (circles), Vpr 62 (triangle), Gag 386 (squares). Cytotoxicity was assayed in a ⁵¹Cr release assay against Jurkat-HLA-A*0201/K^{b} target cells in the presence (filled symbols) or absence (open symbols) of each peptide. (b) Summary of CTL responses of immunogenicity of HIV-FT in HLA-A*0201/K^{b} transgenic mice. Bars indicate the geometric mean CTL response of positive cultures. The frequency of positive CTL cultures is also indicated.

FIG. 5 shows the influence of the C+1 amino acid on epitope immunogenicity. A database incorporating CTL responses from a variety of multi-epitope constructs representing 94 epitope/C+1 amino acid combinations was analyzed to determine the frequency (%) of instances in which a particular combination was associated with an optimal CTL response. CTL responses were considered optimal if greater than 100 SU or 20 LU in at least 30% of the cultures measured. The number of times a given epitope/C+1 amino acid combination was observed is also provided.

FIG. 6 shows CTL responses to HBV-specific constructs (a) CTL responses to Core 18 epitope following DNA immunization of HLA-A*0201/K^{b} transgenic mice. (b) CTL responses to HBV Core 18 following DNA immunization of HLA-A*0201/K^{b} transgenic mice with constructs which vary by a single amino acid insertion at the C+1 position of Core 18.

FIG. 7 shows levels of HBV Core 18 presentation in HBV.1 (shaded bars) and HBV.1K (hatched bars) transfected cell lines. Epitope presentation was quantified using peptide-specific CTL lines. Presentation of HBV Pol 455 is shown for comparative purposes.

FIG. 8 depicts data for 221A2K^{b} target cells transfected with the HIV-FT EpiGene™. These transfected cells were assayed for their capacity to present epitopes to CTL lines derived from HLA transgenic mice and specific for various HIV-derived CTL epitopes. To correct for differences in antigen sensitivity of different CTL lines, peptide dose titrations, using untransfected cells as APC, were run in parallel.

FIG. 9 shows HIV multi-epitope constructs optimized using the methods of the present invention.

FIG. 10 illustrates a computer system for performing automatic optimization of multi-epitope constructs in accordance with one embodiment of the invention.

FIGS. 11A-B illustrate an exemplary input text file containing user input parameters used for executing a Junctional Analyzer program, in accordance with one embodiment of the invention.

FIG. 12 illustrates a flow chart diagram of a software program for identifying optimal multi-epitope constructs, in accordance with one embodiment of the invention.

FIGS. 13A-D illustrate an exemplary output text file containing output results of a Junctional Analyzer program, in accordance with one embodiment of the invention.

FIG. 14A depicts CTL responses induced by EP-HIV-90 relative to individual peptides in IFA, and FIG. 14B depicts CTL responses induced by PfCTL.1, PfCTL.2, and PfCTL.3 relative to individual peptides.

FIG. 15 shows the effect of GPGPG spacers in class II epitope constructs HIV 75 mer and HIV 60 mer on HTL responses to particular epitopes.

FIG. 16 depicts HTL responses to particular epitopes present in the EP-HIV-1043-PADRE® construct.

FIG. 17 is a schematic depicting the epitopes present in HIV 75 mer, EP-HIV-1043, and EP-HIV-1043-PADRE®.

FIGS. 18A-N show the amino acid sequences and nucleic acid sequences of certain multi-epitope constructs.

FIGS. 19A-D show the amino acid sequences for epitopes present in certain multi-epitope constructs.

FIGS. 20A-20F show the HBV CTL epitopes used to construct three related Epi Genes™, HBV-2, HBV-2A and HBV-2B, the order of epitopes in the EpiGenes™, the immune responses induced in HLA-A2 or HLA-A3/11 transgenic mice and the amino acid and nucleic acid sequences of the EpiGenes™. In FIG. 20B, the signal sequence in HBV-2, HBV-2A and HBV-2B is the IgK consensus signal sequence, although other signal sequences could be utilized.

FIGS. 21A-21E show the HBV CTL epitopes used to construct two 21 CTL epitope EpiGenes™, HBV-21A and HBV-21B, the order of epitopes in the EpiGenes™, the immune responses induced in HLA-A2 or HLA-A3/11 transgenic mice and the amino acid and nucleic acid sequences of the EpiGenes™.

FIGS. 22A-22E show the HBV CTL epitopes used to construct two 30 CTL epitope EpiGenes™, HBV-30B and HBV-30C, the order of epitopes in the EpiGenes™, the immune responses induced in HLA-A2 or HLA-A3/11 transgenic mice and the amino acid and nucleic acid sequences of the EpiGenes™.

FIGS. 23A-23C show the modifications made to spacers flanking two HLA-A2 restricted CTL epitopes in the HBV-30C EpiGene™. Modifications were designed to increase the efficiency of processing and subsequent presentation and thus, increase immunogenicity of the epitopes. Immunogenicity was measured using HLA-A2 or HLA-A3/11 transgenic mice, and the amino acid and nucleic acid sequences of the EpiGene™ are noted. In FIG. 23A, the lysine (K) spacer flanking the Core 18 epitope in HBV-30C were modified to include three alanine residues (AAA) in HBV-30CL. Also, one asparagine (N) spacer flanking env 183 epitope in HBV-30C was modified to include three alanine residues (AAA) in HBV-30CL.

FIGS. 24A-24C show HTL epitopes, and their binding affinity to selected HLA-DR alleles, used to construct a multi-epitope vaccine comprising HTL epitopes separated by GPGPG amino acid spacers. The nucleic acid sequence of the multi-epitope vaccine and the amino acid sequence encoded by the nucleic acid are shown in FIG. 24C.

### DETAILED DESCRIPTION OF THE PREFERRED EMBODIMENTS

The invention is described in detail below with reference to the figures wherein like elements are referenced with like numerals throughout. The invention relates to a method and system of designing multi-epitope vaccines with optimized immunogenicity. In preferred embodiments, the vaccine comprises CTL and HTL epitopes. Vaccines in accordance with the invention allow for significant, non-ethnically biased population coverage, and can preferably focus on epitopes conserved amongst different viral or other antigenic isolates. Through the method and system disclosed herein, vaccines can be optimized with regard to the magnitude and breadth of responses, and can allow for the simplest epitope configuration. Finally, general methods are provided to evaluate immunogenicity of a multi-epitope vaccine in humans.

The method of the invention comprises designing a multi-epitope construct based on principles identified herein. In one aspect, the invention provides for simultaneous induction of responses against specific CTL and HTL epitopes, using single promoter multi-epitope constructs. Such constructs can contain many different epitopes, preferably greater than 10, often greater than 20, 25, 30, 25, 40, 45, 50, 55, 60, 65, 70, or more.

In a preferred embodiment, a computer system identifies one or more optimal multi-epitope constructs by performing the following functions and/or analyses:

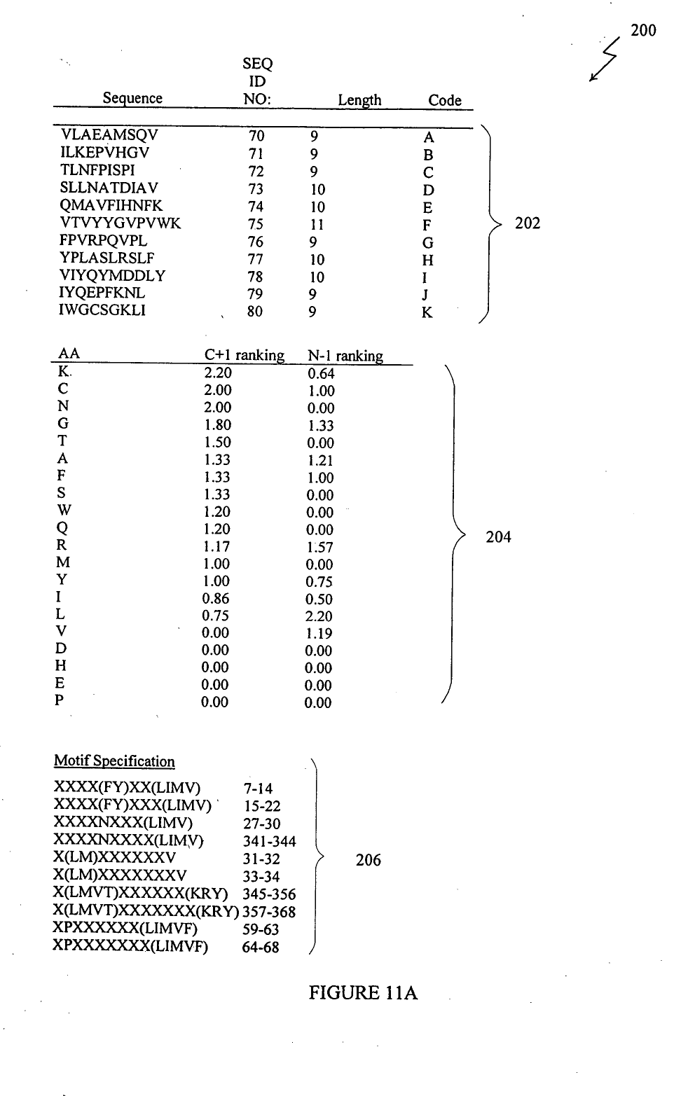

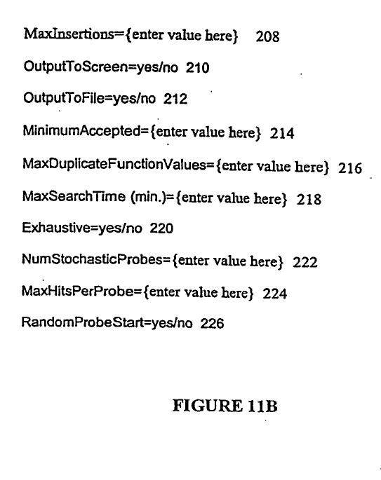

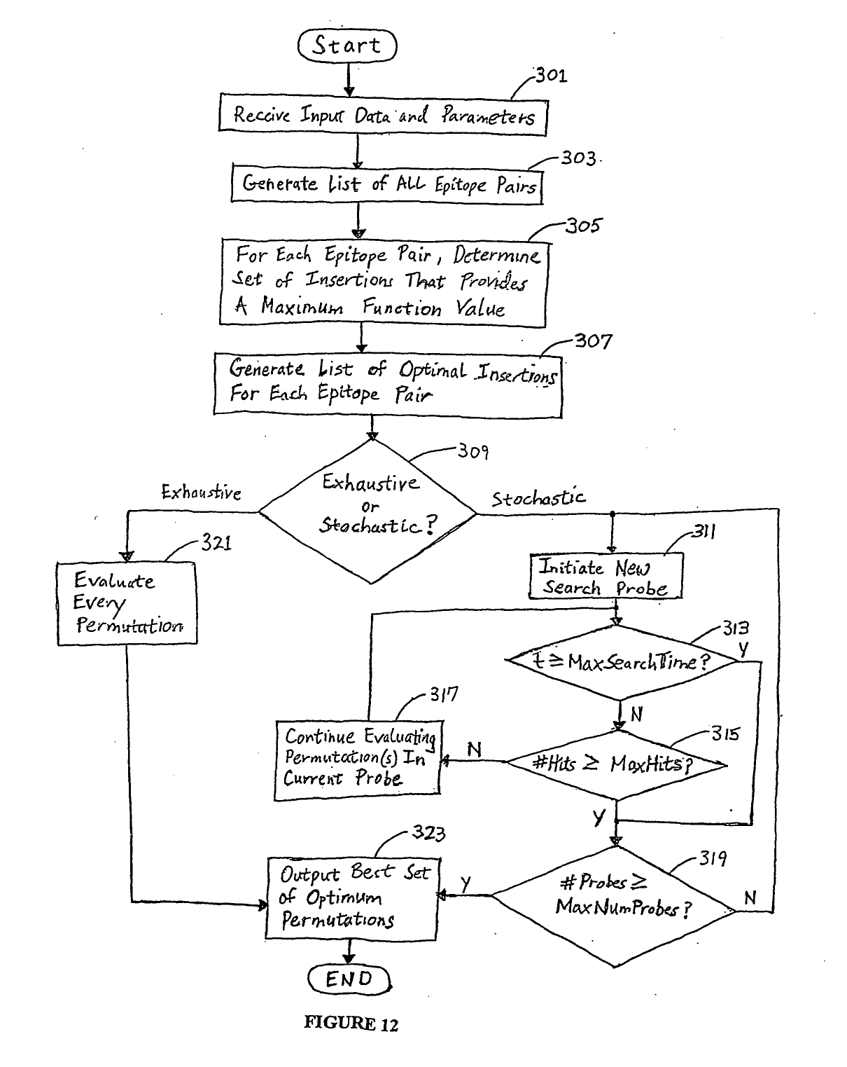

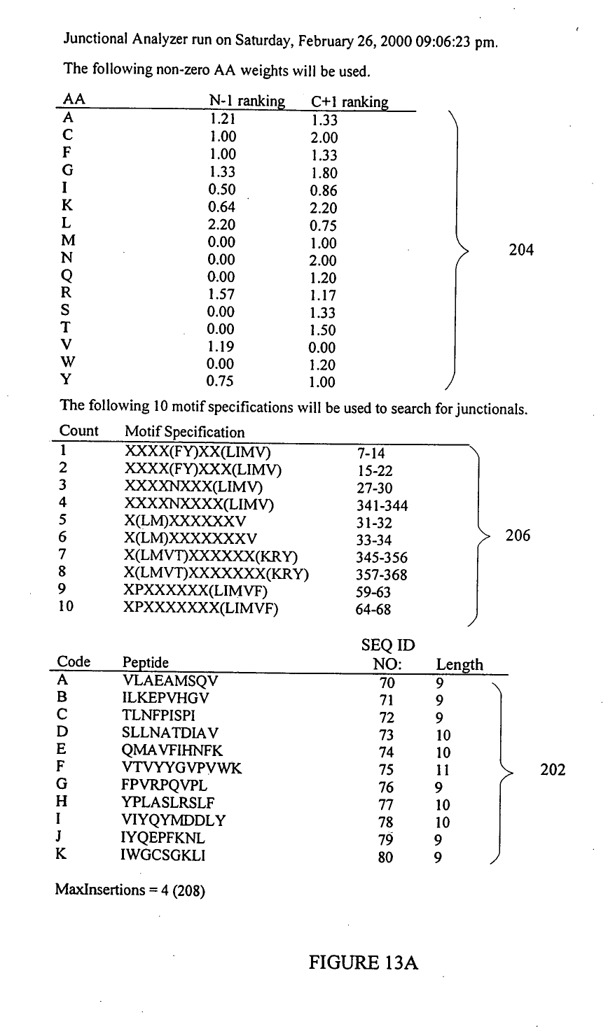

(i) the epitopes to be incorporated into the multi-epitope construct are sorted to provide an order that minimizes the number of junctional epitopes formed. A more detailed discussion of this sorting procedure is provided below with reference to FIGS. 11 and 12. Preferably, as a secondary consideration in ordering epitopes, epitopes are positioned such that residues at the N-terminus of an epitope that promote CTL immunogenicity are juxtaposed to the C-terminus of another CTL epitope. p1 (ii) flanking residues that enhance immunogenicity maybe inserted at the flanking positions of epitopes. In particular embodiments, flanking residues are inserted at the C+1 position of CTL epitopes.

(iii) spacer sequences may be inserted between epitopes to prevent occurance of junctional epitopes. In particular embodiments, the spacer sequences can also include a residue that promotes immunogenicity at the N-terminus of the linker such that the residue flanks the C-terminus of a CTL epitope.

In particular embodiments to prevent HTL junctional epitopes, a spacer composed of amino acid residues that do not correspond to any known HLA Class II anchor residue, are used, e.g, alternating G and P residues (a GP spacer) is included between two HTL epitopes.

Another aspect of the invention, (consideration (ii) above) involves the introduction or substitution of particular amino acid residues at positions that flank epitopes, e.g., a position immediately adjacent to the C-terminus of the epitope, thereby generating multi-epitope constructs with enhanced antigenicity and immunogenicity compared to constructs that do not contain the particular residue introduced or substituted at that site, i.e., non-optimized multi-epitope constructs. The methods of optimizing multi-epitope constructs comprise a step of introducing a flanking residue, preferably K, N, G, R, or A at the C+1 position of the epitope, i.e., the position immediately adjacent to the C-terminus of the epitope. In an alternative embodiment, residues that contribute to decreased immunogenicity, i.e., negatively charged residues, e.g., D, aliphatic residues (I, L, M, V) or aromatic non-trytophan residues, are replaced. The flanking residue can be introduced by positioning appropriate epitopes to provide the favorable flanking residue, or by inserting a specific residue.

As noted in the background section, multi-epitope constructs (minigenes) encoding up to 10 epitopes have been used to induce responses against a number of different epitopes. The data relating to an experimental multi-epitope construct, pMin 0.1 has been published (Ishioka et al., J Immunol, Vol. 162(7):3915-25 (1999)). Disclosed herein, are parameters for designing and evaluating multi-epitope constructs with optimized immunogenicity that address myriad disease indications of interest.

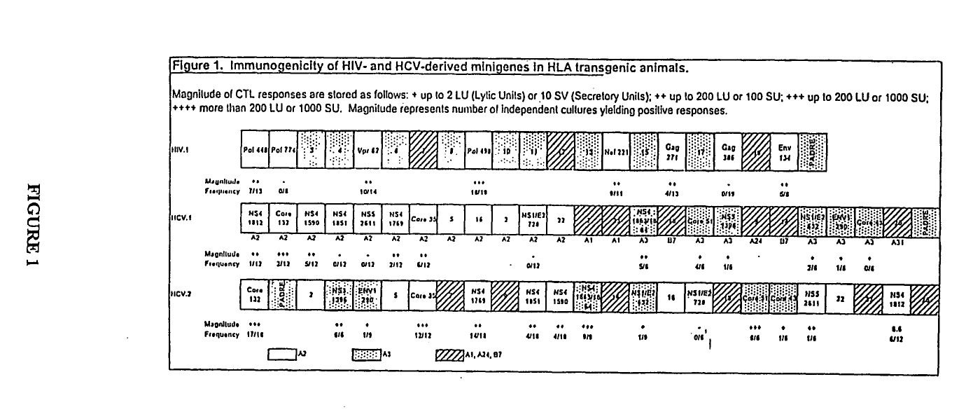

Design parameters were identified based on a number of studies. In a preliminary evaluation of multi-epitope constructs, data on three different multi-epitope constructs, incorporating 20 to 25 different CTL epitopes each, are presented (FIG. 1). One construct is based on HIV-derived epitopes, (HIV-1), while the other two incorporate HCV-derived epitopes (HCV1 and HCV2, respectively). The immunogenicity of these different multi-epitope constructs has been measured in either A2 or A11 HLA transgenic mice (A1, A24 and B7 restricted epitopes were not evaluated).

Thus, eleven days after a single i.m. DNA vaccine injection, responses against 8 to 14 different representative epitopes were evaluated following a single six day in vitro restimulation, utilizing assays to measure CTL activity (either chromium release or in situ IFN production, as described herein). Priming of epitope specific CTL could be demonstrated for 6/8 (75%), 10/14 (72%) and 13/14 (93%) of the epitopes tested in the case of HIV-1, HCV1 and HCV2, respectively. Thus, multi-epitope constructs, capable of simultaneously priming CTL responses against a large number of epitopes, can be readily designed. However, it should be emphasized that CTL priming for some epitopes was not detected and, in several of the 36 cases considered, responses were infrequent, or varied significantly in magnitude over at least three orders of magnitude (1000-fold). These results strongly suggested that a more careful analysis and optimization of the multi-epitope constructs was required.

The possibility that the suboptimal performance of priming for certain epitopes night be related to multi-epitope construct size was also examined. In fact, most of the published reports describe multi-epitope construct of up to ten epitopes, and the few instances in which 20-epitope constructs have been reported, activity directed against only two or three epitopes was measured. To address this possibility, two smaller EpiGenes™(HIV-1.1 and HIV-1.2) each encompassing ten epitopes, and corresponding to one half of the HIV-1 EpiGene™, were synthesized and tested. Responses against four representative epitopes were measured.

TABLE 1
Immunogenicity appears to be independent of EpiGene ™ size.

| | CTL response to different EpiGenes ™ | | | | | |
|---|---|---|---|---|---|---|
| | HIV 1 | | HIV 1.1 | | HIV 1.2 | |
| | (20 mer) | | (10 mer) | | (10 mer) | |
| CTL | Fre- | Mag- | Fre- | Mag- | Fre- | Mag- |
| Epitope | quency¹⁾ | nitude² | quency | nitude | quency | nitude |
| Pol 774 | 0/8 | * | 0/4 | * | NA³⁾ | NA |
| Pol 498 | 18/19 | 46.7 | 4/4 | 16.4 | NA | NA |
| Gag 271 | 4/13 | 4.0 | NA | NA | 0/4 | * |
| Env 134 | 5/8 | 16.1 | NA | NA | 4/4 | 14.8 |

¹⁾Represents the fraction of independent cultures yielding positive responses
²⁾Lytic Units (LU)
³⁾Not Applicable

It was found that the responses induced by the smaller EpiGenes™ were comparable, and if anything, lower than those induced by the twenty-epitope construct (Table 1). Accordingly, factors relating to EpiGene™ size are unlikely explanations for the observed suboptimal priming to certain epitopes and thus other parameters, disclosed herein, are used to design efficacious multi-epitope constructs.

The Minimization of Junctional Motifs

One of the considerations in designing multi-epitope constructs is the inadvertent creation of junctional epitopes when placing epitopes adjacent to each other. The presence of such epitopes in a multi-epitope construct could significantly affect performance. Strategies to guard against this undesired effect are disclosed herein for application to the development of multi-epitope vaccines. Junctional epitopes can first be minimized by sorting the epitopes to identify an order in which the numbers of junctional epitopes is minimized. Such a sorting procedure can be performed using a computer or by eye, if necessary, or depending on the number of epitopes to be included in the multi-epitope construct.

For example, a computer program that finds patterns, e.g., Panorama, manufactured by ProVUE Development, Huntington Beach, Calif., U.S.A., can be used in accordance with one embodiment of the invention. A very large number of different epitope arrangements can be considered in designing a particular multi-epitope construct. A computer program accepts as input, the particular set of epitopes considered, and the motifs to be scanned in order to evaluate whether there are any junctional epitopes bearing these motifs. For example, a program can simulate building a multi-epitope construct, and in an euristic computational algorithm, examine epitope pairs to avoid or minimize the occurrance of junctional motifs. The program can for example, evaluate 6×105 (about half a million) multi-epitope construct configurations/second.

A complete analysis of a 10-epitope construct using a computer program as described in the preceding paragraph requires examining 10 factorial≅3.6×10⁶ combinations and can be completed in six seconds. A fourteen-epitope construct can be completely analyzed in a couple of days. Thus, analysis time goes up very rapidly as larger constructs are considered. However, a complete analysis is not always required and the program can be run for any desired length of time. In either case, the computer system of the present invention identifies and provides at least one configuration having a minimum number of junctional epitopes.

An example of the results of this type of approach is presented in Table 2. The number of junctional motifs in ten different random assortments of the same epitopes contained in the HCV1 EpiGene™, which incorporates 25 epitopes, and is the result of a two day computer analysis, is presented in this Table. In the non-optimized assortments, a large number of A2, A11 and K^{b} motifs were found, in the 25 to 38 range, with an average of 31. By comparison, only two such junctional motifs are present in the HCV1 EpiGene™ assortment. In conclusion, a computer program can be utilized to effectively minimize the number of junctional motifs present in multi-epitope constructs.

TABLE 2
Occurrence of junctional epitopes.

| EpiGene ™ | selection criteria | junctional motifs |
|---|---|---|
| HCV.a | random | 33 |
| HCV.b | random | 26 |
| HCV.c | random | 28 |
| HCV.d | random | 27 |
| HCV.e | random | 30 |
| HCV.f | random | 26 |
| HCV.g | random | 38 |
| HCV.h | random | 33 |
| HCV.i | random | 33 |
| HCV.j | random | 34 |
| HCV.1 | minimized | 2 |

Eliminating Class II Junctional Epitopes and Testing for Class II Restricted Responses In Vivo

As a further element in eliminating junctional epitopes, spacer sequences can be inserted between two epitopes that create a junctional epitope when juxtaposed.

In one embodiment, to correct the problem of junctional epitopes for HTL epitopes, a spacer of, for example, five amino acids in length is inserted between the two epitopes. The amino acid residues incorporated into such a spacer are preferably those amino acid residues that are not known to be primary anchor residues for any of the HLA Class II binding motifs. Such residues include G, P, and N. In a preferred embodiment, a spacer with the sequence GPGPG is inserted between two epitopes. Previous work has demonstrated that the GP spacer is particularly effective in disrupting Class II binding interactions (Sette et al., J. Immunol., 143:1268-73 (1989)). All known human Class II binding motifs and the mouse IA^{b} (the Class II expressed by HLA transgenic mice) do not tolerate either G or P at this main anchor positions, which are spaced four residues apart. This approach virtually guarantees that no Class II restricted epitopes can be formed as junctional epitopes.

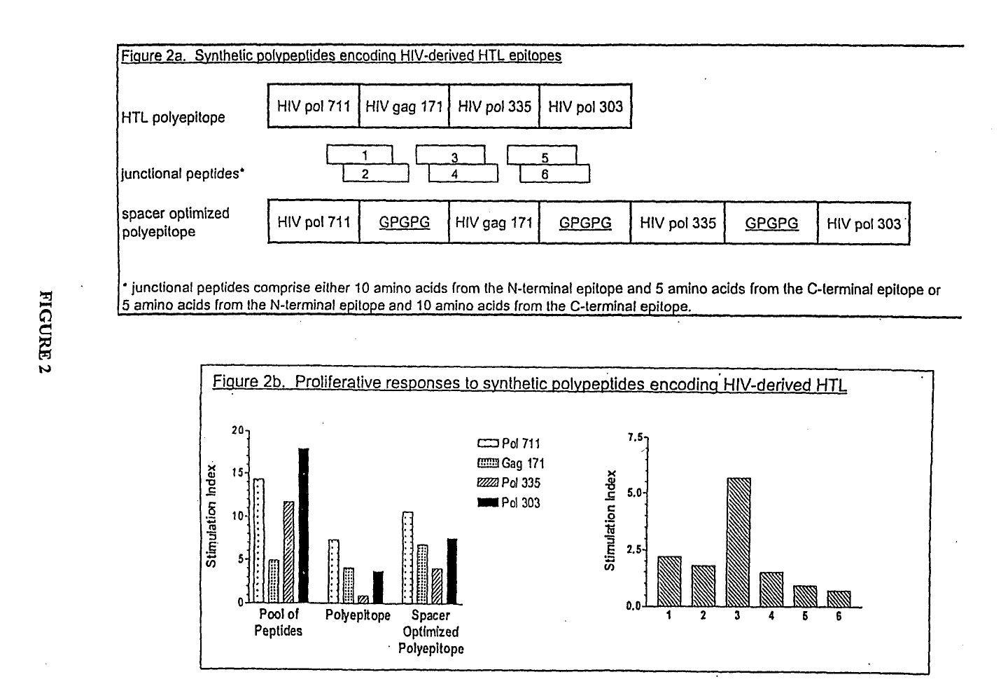

In an example validating this design consideration, we synthesized polypeptides incorporating HIV-derived HTL epitopes. These epitopes are broadly cross-reactive HLA DR binding epitopes. It was then determined that these epitopes also efficiently bind the murine IA^{b} Class II molecule. A diagram illustrating the two different synthetic polypeptides considered is shown in FIG. 2a.

The first construct incorporates four different epitopes linearly arranged, while the second construct incorporates the GPGPG spacer. Synthetic peptides corresponding to the three potential junctional epitopes were also synthesized.

The capacity of 2 nanomoles of these different constructs to prime for proliferative responses to the various epitopes in IA^{b} positive mice was tested, and compared to the responses induced by equimolar amounts of a pool of the same peptides (3 micrograms of each peptide). Specifically, groups of 3 mice were injected with peptides in CFA emulsions, 11 days after injection their lymph node cells were cultured in vitro for an additional 3 days, and thymidine incorporation was measured in the last 24 hours of culture. It was found (FIG. 2b) that, as predicted on the basis of their high affinity IA^{b} binding capacity, all four epitopes induced good proliferation responses. Stimulation index (SI) values in the 4.9 to 17.9 range were observed when these peptides were injected in a pool. However, when the linear polypeptide incorporating the same epitopes was tested, the response directed against Pol 335 was lost. This was associated with appearance of a response directed against a junctional epitope spanning Gag 171 and Pol 335. The use of the GPGPG spacer eliminated this problem, presumably by destroying the junctional epitope, and the Pol 335 response was regained. The responses observed were of magnitude similar to those observed with the pool of isolated peptides.

These results demonstrate that responses against multiple HIV-derived Class II epitopes can be simultaneously induced, and also illustrate how IA^{b}/DR crossreactivity can be utilized to investigate the immunogenicity of various constructs incorporating HTL epitope candidates. Finally, they demonstrate that appropriate spacers can be employed to effectively disrupt Class II junctional epitopes that would otherwise interfere with effective vaccine immunogenicity.

In the case of Class I restricted responses, one case of a naturally occurring junctional epitope and the consequent inhibition of epitope specific responses has been presented by McMichael and coworkers (Tussey et al., Immunity, Vol. 3(1):65-77 (1995)). To address the problem of junctional epitopes for Class I, similar analyses can be performed. For example, a specific computer program is employed to identify potential Class I restricted junctional epitopes, by screening for selected murine motifs and for the most common human Class I HLA A and B motifs.

Spacer sequences can also similarly be employed to prevent CTL junctional epitopes. Often, very small residues such as A or G are preferred spacer residues. G also occurs relatively infrequently as a preferred primary anchor residue (see, e.g., PCT/US00/24802) of an HLA Class I binding motif. These spacers can vary in length, e.g., spacer sequences can typically be 1, 2, 3, 4, 5, 6, 7, or 8 amino acid residues in length and are sometimes longer. Smaller lengths are often preferred because of physical constraints in producing the multi-epitope construct.

The Influence of Flanklng Regions on CTL Multi-Epitope Construct Immunogenicity

Another factor to be considered in designing multi-epitope constructs is to insert residues that favor immunogenicity at the position flanking the C-terminus of a CTL epitope.

Disclosed herein are studies that identify residues that increase immunogenicity and, accordingly, residues that are inserted in multi-epitope constructs to optimize immunogenicity.

The molecular context in which an epitope was expressed often dramatically influenced the frequency and/or magnitude of priming of CTL specific for that epitope in HLA transgenic mice. Two examples are shown in Table 3.

TABLE 3

Differences in effectiveness of T cell priming for
specific epitopes in different
EpiGenes ™.

| | | | | | | |
|---|---|---|---|---|---|---|
| | | Flanking | | Flanking | Immune | Immune |
| Epitope | EpiGene ™ | Sequence | Epitope | Sequence | Response | Response |

| | | | | | | |
|---|---|---|---|---|---|---|
| Identity | Identity | (N terminus) | Sequence | (C-terminus) | Frequency | Magnitude¹⁾ |

| | | | | | | |
|---|---|---|---|---|---|---|
| Core 18 | HBV.1 | TLKAAA | FLPSDFFPSV | FLLSLG | 6/6 | 5.5 |
| | pMin1 | TLKAAA | FLPSDFFPSV | KLTPLC | 6/6 | 1074.5 |
| Core 132 | HCV1 | ILGGWV | DLMGYIPLV | YLVAYQ | 2/12 | 107.7 |
| | HCV2 | VPGSRG | DLMGYIPLV | AKFVA | 17/18 | 929.2 |

¹⁾IFNγ secretory units

The immunogenicity of the HBV Core 18 epitope expressed in the pMin5 EpiGene™ was approximately 200-fold lower in magnitude than that observed in the case of the pMin1 EpiGene™. Similarly, the immunogenicity of the HCV Core 132 epitope expressed in the context of the HCV1 EpiGene™ was marginal, with significant T cell priming demonstrable in only 2 of 12 different independent CTL experiments/cultures performed. These two positive experiments yielded responses of approximately 100 SU of IFNγ. However, when the same epitope was expressed in the context of the HCV2 EpiGene™, positive responses were observed in 17/18 cases, and with average magnitudes approximately five-fold higher.

Immunogenicity of HIV-FT in HLA-A*0201/Kb Transgenic Mice

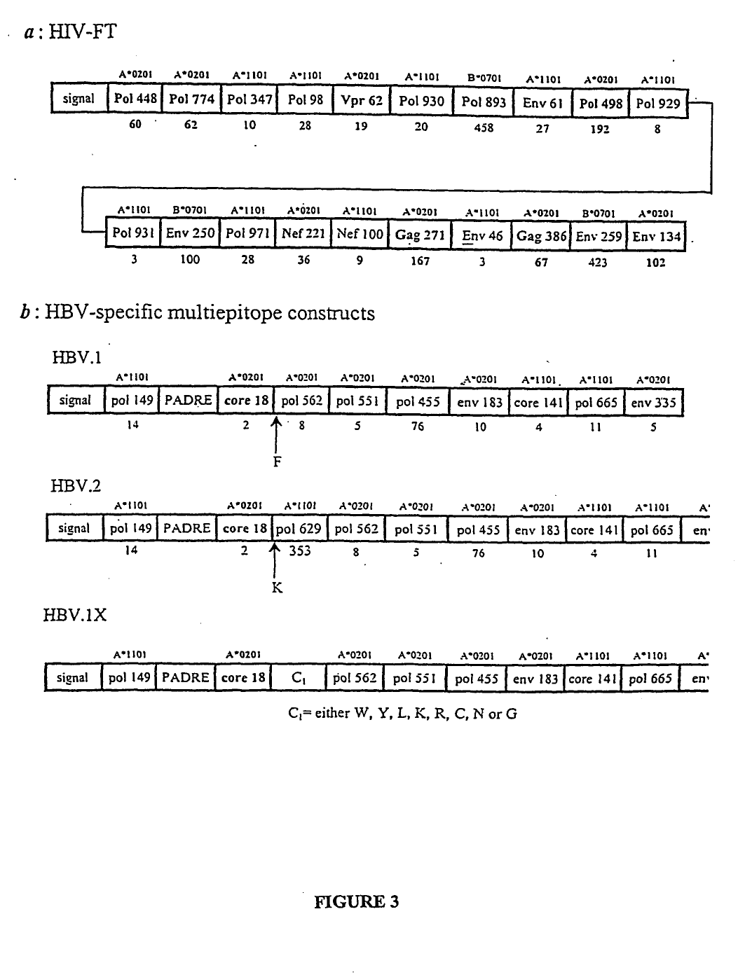

An HIV multi-epitope DNA vaccine, HIV-FT (FIG. 3a) encodes 20 HIV-derived CTL epitopes. Of these 20 epitopes, eight are restricted by HLA-A*0201, nine by HLA-A*1101 and three by HLA-B*0702. All epitopes bound their relevant restriction element with high or moderate affinity. All of the HLA-A*0201 restricted epitopes bound purified HLA-A*0201 molecules with roughly similar affinities, with IC₅₀ values in the 19-192 nM range (FIG. 3a). The HLA-A*0201 epitopes chosen for inclusion in HIV-FT are recognized in HIV-1 infected individuals and were also highly effective in priming for recall CTL responses when emulsified with IFA and utilized to prime HLA-A*0201/K^{b} transgenic mice. The construct was designed to encode the epitopes sequentially without any intervening spacer sequences between them and a consensus IgK signal sequence was fused to the 5′ end of the construct to facilitate transport of the encoded antigen into the endoplasmic reticulum (Ishioka et al., J. Immunol. 162:3915-3925, 1999).

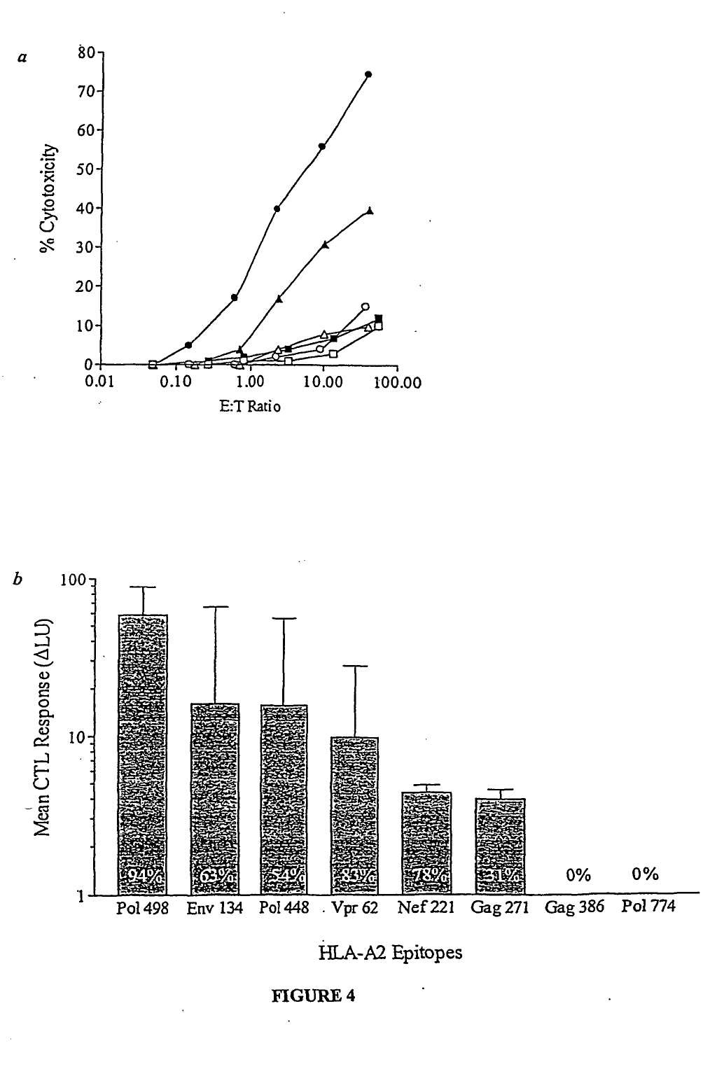

The ability of HIV-FT to prime recall CTL responses in vivo was evaluated by intramuscular immunization of HLA-A*0201/K^{b} transgenic mice. Splenocytes from animals immunized with 100 μg of HIV-FT plasmid DNA were stimulated with each of the HLA-A*0201 epitopes encoded in HIV-FT and assayed for peptide-specific CTL activity after six days of culture. Representative CTL responses against three of the epitopes in HIV-FT are shown in FIG. 4a. To more conveniently compile results from different experiments the percent cytotoxicity values for each splenocyte culture were expressed in lytic units (Vitiello, et al., J. Clin. Invest 95:341-349, 1995). Of the eight HLA-A*0201 restricted epitopes encoded in HIV-FT, Pol 498, Env 134, Pol 448, Vpr 62, Nef 221, and Gag 271, primed for CTL responses following DNA immunization, (FIG. 4b). The magnitude of the CTL responses varied over greater than a 10-fold range, from as high as nearly 50 LU against Pol 498, too as little as 4 LU against Nef221 and Gag 271. Similarly, the frequency of recall CTL responses varied between epitopes, with the Pol 498 epitope inducing responses in 94% of the experiments while CTL responses to Gag 271 were detected in only 31% ofthe experiments. In conclusion, DNA immunization with HIV-FT; which sequentially encodes the epitopes without any spacer amino acids, induced recall CTL responses against the majority of the epitopes analyzed. However, the magnitude and the frequency of the responses varied greatly between epitopes.

Correlation Between Epitope Immunogenicity and Levels of HIV-FT Epitope Presentation in Transfected Cell Lines

The differential immunogenicity of the HLA-A*0201 epitopes in HIV-FT was then assessed. Differential MHC binding affinity could be excluded as all of the epitopes bind HLA-A*0201 with high affinity (FIG. 3a). In addition, lack of a suitable repertoire of TCR specificities in HLA-A*0201/K^{b} transgenic mice could be excluded since all epitopes yielded comparable CTL responses following immunization of HLA transgenic mice with the optimal preprocessed peptide emulsified in IFA. Variations in the relative amounts of each epitope presented for T cell recognition may account for the differences in epitope immunogenicity.

To test this, Jurkat cells, a human T cell line, expressing the HLA-A*0201/K^{b} gene (Vitiello et al., J. Exp. Med. 173, 1007-1015, 1991) were transfected with the HIV-FT expressed in an episomal vector. A human cell line was selected for use to eliminate any possible artifacts that may be associated with potential differences in the processing capabilities between humans and mice. This transfected cell line matches the human MHC presentation with human antigen processing capabilities and provides support for the subsequent development of CTL epitope-based DNA vaccines for use in humans.

Peptide-specific CTL lines detected presentation in the transfected targets of four of the HLA-A*0201 epitopes encoded in the HIV-FT, Pol 498, Env 134, Pol 448 and Nef 221. To quantitate the level at which each of these epitopes was produced and presented, the CTL lines specific for the various epitopes were incubated with untransfected targets and variable amounts of each epitope or peptides. These CTL dose response curves were utilized as standard curves to determine the peptide concentration inducing levels of IFNγ secretion equivalent to those observed in response to the HIV-FT transfected target cells. This value is referred to as a “peptide equivalent dose” and taken as a relative measure of the amount of epitope presented on the transfected cell.

Table 4 summarizes the findings of this analysis for eight of the HLA-A*0201 epitopes encoded in the HIV-FT. Peptide equivalent doses varied from a high of 33.3 ng/ml for Nef 221 to less than 0.4 ng/ml peptide equivalents for epitopes Gag 271, Gag 386 and Pol 774. Cumulatively these results indicate that in human cells lines transfected with HIV-FT there is at least a 100-fold variation exists in the levels of presentation of the different HLA-A*0201 restricted epitopes. All of the epitopes that were presented at detectable levels in antigenicity assays were also immunogenic in vivo. The only epitope that was immunogenic and not antigenic was Gag 271. In this case, immunization of HLA-A*0201/Kb transgenic mice with HIV-FT induced a weak CTL response in less than a third of the cultures tested. The other two epitopes, which were presented below the limit of sensitivity for the antigenicity analysis, Gag 386 and Pol 774, were non-immunogenic. In conclusion these results suggest that the heterogeneity in CTL responses induced by HIV-FT immunization can at least in part be attributed to suboptimal epitope presentation.

TABLE 4
Comparison of HIV-FT immunogenicity and antigenicity

| | |
|---|---|
| | HIV-FT |
| HIV-FT | Antigenicity |

| | Immunogenicity | | Peptide | |
|---|---|---|---|---|
| Epitope | magnitude¹ | frequency² | Equivalents³ | n⁴ |
| Pol 498 | 58.8 (2.2) | 94% (16/17) | 23.8 (2.0) | 4 |
| Env 134 | 16.1 (5.0) | 63% (5/8) | 6.2 (1.2) | 3 |
| Pol 448 | 15.7 (2.6) | 54% (7/13) | 24.7 (3.9) | 3 |
| Vpr 62 | 9.9 (1.9) | 83% (10.12) | ND | — |
| Nef 221 | 4.4 (1.3) | 78% (7/9) | 33.3 (6.0) | 3 |
| Gag 271 | 4.0 (1.4) | 31% (4/13) | <0.4 | 6 |
| Gag 386 | 0 | 0% (0/17) | <0.4 | 3 |
| Pol 774 | 0 | 0% (0/8) | <0.4 | 1 |

¹magnitude expressed as LU (ref); the correlation coefficient relative to peptide equivalents R + 0.44
²frequency of positive cultures (number cultures >2LU/total tested); the correlation coefficient relative to peptide equivalents R + 0.8.
³magnitude expressed in ng/ml
⁴number of independent experiments

Flanking Amino Acids Influence CTL Epitope Immunogenicity In Vivo Following Vaccination

As described herein, the particular amino acids flanking individual CTL epitopes is one factor that influences or enhances the efficiency with which an epitope is processed by altering the susceptibility of the antigen to proteolytic cleavage. To examine the influence of flanking amino acids on epitope immunogenicity, immunogenicity data was obtained from HLA-A*0201, -A*1101 and -B*0701 transgenic mice immunized with a number of unrelated experimental multi-epitope DNA constructs encoding minimal CTL epitopes without intervening sequences. A database representing 94 different epitope/flanking residue combinations was compiled to determine the possible influence the immediately flanking amino acids on epitope immunogenicity. A given epitope and flanking amino acid combination was included only once to prevent artificial skewing of the analysis because of redundancies. Epitope immunogenicity in HLA transgenic was considered optimal if greater than 100 SU or 20 LU in at least 30% of the cultures measured. CTL responses were typically scored in one of four categories: (+++), outstanding-more than 200 LU or 1000 SU; (++), good-20-200 LU or 100-1000 SU; (+), intermediate-2 to 20 LU or 10 to 100 SU; and (+/−), weak or negative-less than 2 LU or 10 SU. The numbers of optimal versus sub-optimal responses were categorized based on the chemical type of amino acid in the flanking positions and the significance of differences were determined using a chi-square test.

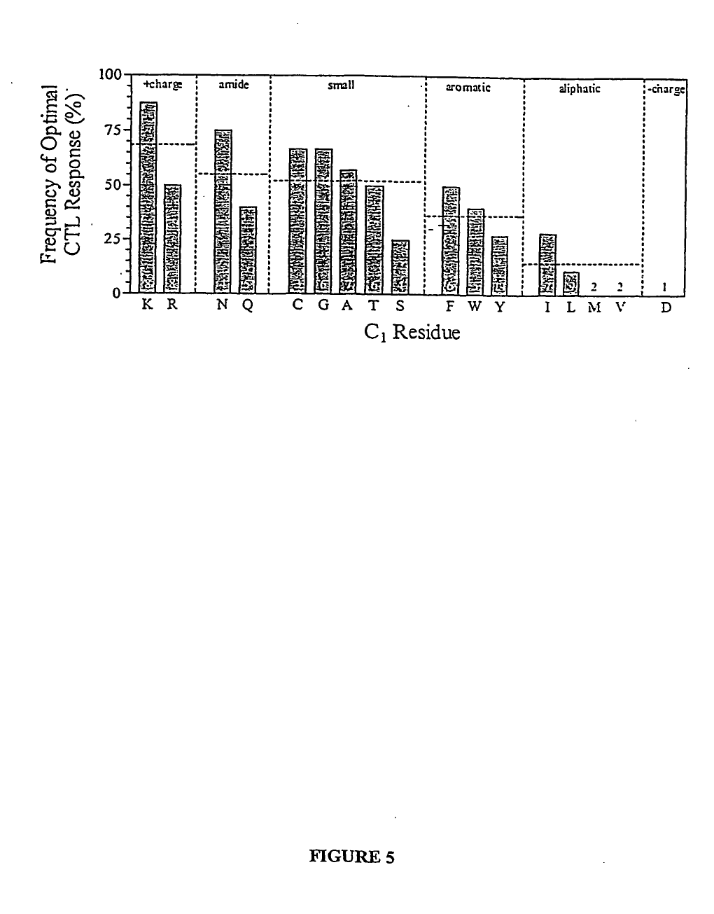

This analysis did not find any associations between the type of amino acids present at the amino-terminus of an epitope and immunogenicity. However, significant effects of the carboxyl-terminus flanking residue, the C+1 residue, were identified. Positively charged amino acids, K or R were most frequently associated with optimal CTL responses, a frequency of 68% (FIG. 5). The presence of amino acids N and Q at the C+1 residue was also associated with strong CTL responses in 55.5% of the cases examined; when epitopes were flanked at the C+1 position by N, they induced optimal CTL responses in 3/4 cases. In general, small residues such as C, G, A, T, and S promoted intermediate CTL responses inducing strong responses in 54% of the combinations available for analysis. Conversely, epitopes flanked by aromatic and aliphatic amino acids induced optimal in vivo responses in only 36% and 17% of the cases, respectively. The negatively charged residue, D, yielded a suboptimal CTL response. The influence of C+1 amino acid on epitope immunogenicity was found to be statistically significant using a chi-square test (P<0.03). No significant influence on epitope immunogenicity was noted when similar analysis was performed for C-terminal residues more distal than the C+1 position.

Direct Evaluation of the Effect of the C1 Residue on Epitope Immunogenicity

To directly evaluate the effect of preferred versus deleterious types of amino acids in the C+1 flanking position, two multi-epitope constructs, referred to as HBV.1 and HBV.2 (FIG. 3b) were evaluated. As with HIV-FT, these HBV constructs encode the epitopes sequentially without intervening spacers. Indeed, the HBV.1 and HBV.2 were generated by replacing the HIV-1 epitopes in pMin1, an experimental multi-epitope construct previously characterized (Ishioka, supra) with similar HBV-derived epitopes.

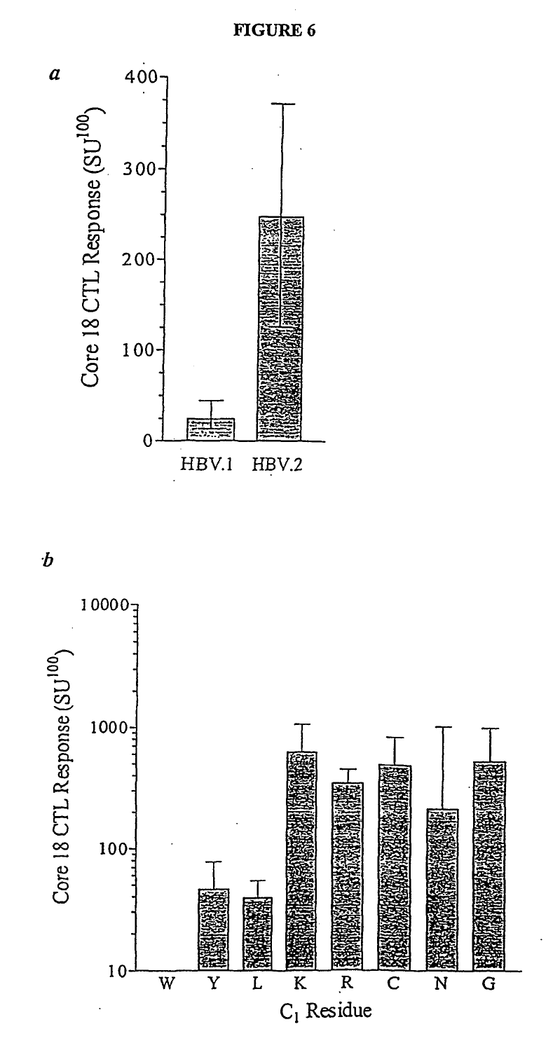

For HBV.1, the HIV-1 epitope directly following the highly immunogenic HBV Core 18 epitope was replaced with the HBV Pol 562 epitope. This altered the C+1 residue from a K to an F. The second construct, HBV.2, was produced by the insertion of an additional epitope, HBV Pol 629, between the HBV Core 18 and Pol 562 epitopes; a change that replaced the C+1 amino acid with a K residue. When the immunogenicity of the Core 18 epitope presented in these different contexts was evaluated in HLA-A*0201/K^{b} transgenic mice, it was determined that the Core 18 epitope was virtually non-immunogenic in HBV.1 but strongly immunogenic in HBV.2 (FIG. 6a). The reduction of in vivo immunogenicity for this epitope was as would be predicted by our previous analysis.

To further test the effects of the C+1 flanking amino acid on CTL epitope immunogenicity, a set of constructs that differ from HBV.1 by the insertion of single amino acids at the C+1 position relative to the Core 18 epitope (FIG. 3b) was evaluated. Little or no CTL response was observed against the Core 18 epitope when flanked at the C+1 position by W, Y, or L (FIG. 6b). In contrast, insertion of a single K residue dramatically increased the CTL response to Core 18. The responses were comparable to those observed in HBV.2 in which the Core 18 epitope is flanked by Pol 629, an epitope with a K at the N-terminus of the epitope. Enhancement of the Core 18 CTL response was also observed to insertion of R, C, N, or G. The effect of these insertions is specific, as the immunogenicity of other epitopes within these constructs did not exhibit significant changes in CTL responses (data not shown). In conclusion, these data indicate that the C+1 amino acid can dramatically influence epitope immunogenicity.

Variations in CTL Epitope Immunogenicity are Correlated with the Amount Presented

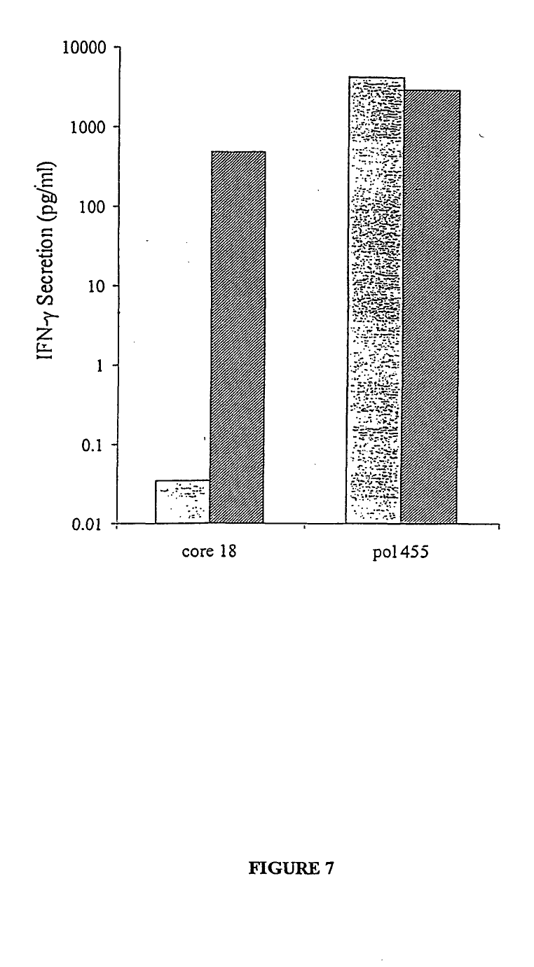

If the variation of the immunogenicity of Core 18 associated with different C+1 residues was the result of differential sensitivity to proteolytic cleavage then large differences in the levels of epitope presentation should be detectable in different constructs. To test this, Jurkat cells, expressing the same HLA-A*0201/K^{b} gene expressed in the transgenic mice, were transfected with an episomal vector expressing either HBV.1 or HBV.1K. The Core 18 epitope was presented at>10⁵ higher levels when a K was in the C+1 position, compared to the presence of an F in the same position (FIG. 7). It is unlikely that this difference in Core 18 presentation is attributed to differences in gene expression between target cell lines since presentation of Pol 455 varied by less than ten-fold. These data demonstrate the striking effect that amino acids at the C+1 position can exert on efficiency of epitope presentation in multi-epitope DNA vaccines. Thus, these data show that the immunogenicity of CTL epitopes in a DNA vaccine can be optimized through design considerations that affect the level of epitope presentation. This type of optimization is applicable to epitope-based vaccines delivered using other formats, such as viral vectors as well as other expression vectors known to those of skill in the art, since the effects are exerted after the antigen is transcribed and translated.

In summary, for flanking residues, it was found that either very small residues such as A, C or G, or large residues such as Q, W, K, or R were generally associated with good or outstanding responses. The absence of a C+1 residue because of a stop codon in the multi-epitope construct, or the presence of intermediate size residues such as S or T was associated with a more intermediate response pattern. Finally, in the case of a negatively charged residue, D; aliphatic (V, I, L, M) or aromatic-non tryptophan residues (Y, F), relatively poor responses were observed. These results show that the particular residue flanking the epitope's C-terminus can dramatically influence the response frequency and magnitude. Flanking residues at the C+1 position can also be introduced in combination with spacer sequences. Thus, a residue that favors immunogenicity, preferably, K, R, N, A, or G, is included as a flanking residue of a spacer.

Sorting and Optimization of Multi-Epitope Constructs

To develop multi-epitope constructs using the invention, the epitopes for inclusion in the multi-epitope construct are sorted and optimized using the parameters defined herein. Sorting and optimization can be performed using a computer or, for fewer numbers of epitopes, not using a computer.

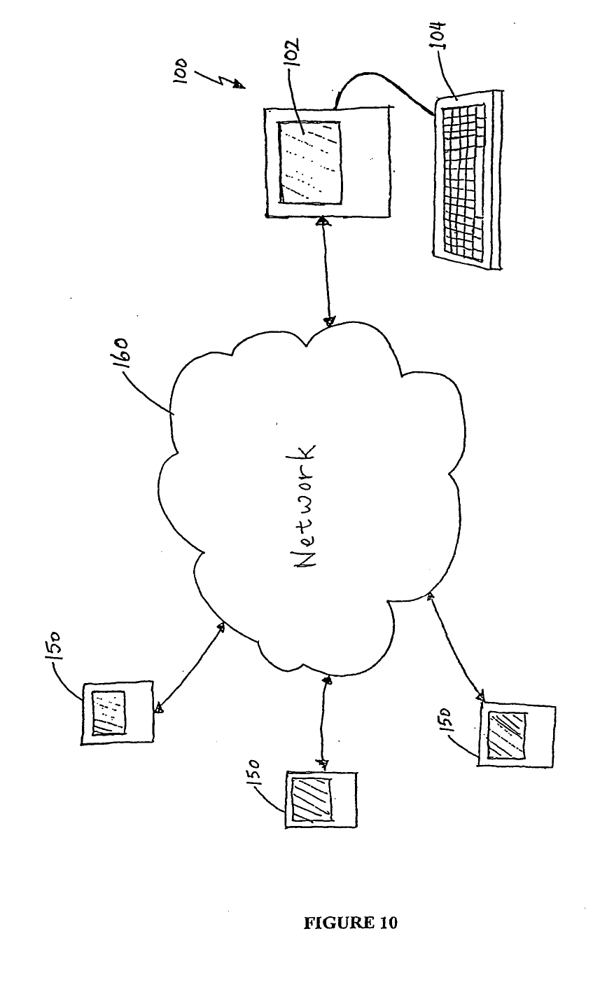

Computerized optimization can typically be performed as follows. The following provides an example of a computerized system that identifies and optimizes, e.g., provides for a minimal number of junctional epitopes and a maximal number of flanking residues, epitope combinations. FIG. 10 illustrates a computer system 100 for performing the optimization of multi-epitope constructs, in accordance with one embodiment of the invention. The computer system 100 may be a conventional-type computer which includes processing circuitry, e.g., a central processing unit (CPU), memory, e.g., a hard disk drive (ROM), a random access memory (RAM), cache, and other components, devices and circuitry (not shown) typically found in computers today. In a preferred embodiment, the computer system 100 includes, among other components and devices, a Macintosh G3 333 MHz processor, a six Gigabit (GB) hard drive, 96 Megabits of RAM, and 512 Kilabits (KB) of cache memory, capable of searching 600,000 to 700,000 permutations per second. The computer system 100 further includes a monitor 102 for displaying text, graphics and other information to a user and a keyboard 104 for allowing a user to input data, commands, and other information to the computer system 100.

As shown in FIG. 10, in one embodiment, the computer system 100 may communicate with one or more remote computers 150 through a computer network 160 such that registered users at remote locations can perform the junctional analyses and multi-epitope construct optimization procedures described herein by logging on at the remote computers 150 and supplying a required password or user identification. The computer network 160 may be a local area network (LAN), a wide area network (WAN), or the world-wide web (i.e., Internet). These types of networks are well-known in the art and, therefore, a discussion of these networks and their communication protocols is not provided herein.

In a preferred embodiment, the computer system 100 stores a software program, e.g., object code, in the hard drive memory of the computer system 100. This object code is executed by the CPU for performing the functions described herein. One component, or module, of the software program carries out the function of analyzing and identifying junctional epitopes at the peptide junctions of the polypeptide encoded by a multi-epitope nucleic acid construct as well as evaluating combinations of spacer and flanking residues at these junctions. This software module is referred to herein as the “Junctional Analyzer” module or program. In a preferred embodiment, the Junctional Analyzer analyzes and processes peptides entered by a user in accordance with other criteria, data and operating parameters described below.

FIGS. 11A-B (hereinafter FIG. 11) illustrate an exemplary input text file 200 containing user input data and parameters which is used by the Junctional Analyzer program, in accordance with one embodiment of the invention. As shown in FIG. 11, various types of input data are provided to the program. First, a user may enter a set of peptides or epitopes 202 for processing. A set of weights 204 for each amino acid when it appears in a C+1 and N−1 position is also entered into the text file by the user. In one embodiment, the weight values are determined by statistical or empirical analysis of experimental results which reflect the immunogenicity or antigenicity “enhancement” effects of each amino acid when it is placed at the C+1 or N−1 positions of a polypeptide. However, the assignment of weight values for each amino acid may be performed by any number of methodologies, including in vitro and in vivo studies, which would be apparent to those of ordinary skill in the art, depending on the desired criteria used to determine the weight values. Some examples of such experiments or studies are described in further detail below.

In a preferred embodiment, a database containing different epitope/flanking residue combinations is stratified on the basis of epitope immunogenicity and the number of optimal versus suboptimal responses are sorted to rank the amino acids and assign enhancement weight values. The text file also contains a set of motifs 206 to use in detecting junctional epitopes. In a preferred embodiment, the user may also enter a maximum number of amino acids (spacers and flanking) to insert between each pair of peptides (MaxInsertions) 208 to function as spacers and/or flanking residues. Other parameters, values or commands (collectively referred to herein as “parameters”) to control the operation of the program may also be entered such as, for example: “OutputToScreen (Y/N)” 210; “OutputToFile (Y/N)” 212; the minimum function value to accept as a valid result (“MinimumAccepted”) 214; the maximum number of results having the same function value (“MaxDuplicateFunctionValue”) 216; the maximum time allowed for a search in minutes (“SearchTime”) 218; whether an Exhaustive Search is desired (“Exhaustive=Y/N”) 220; the number of Stochastic search probes (“NumStochasticProbes”) 222; the maximum number of hits allowed per single probe during a stochastic search (“MaxHitsPerProbe”) 224; and whether the start of each probe should be random or other (“RandomProbeStart(Y/N)”) 226. These parameters are provided for purposes of illustration only. Other parameters to control the operation and output format of the program may be entered as would be obvious to those of ordinary skill in the art.

The motifs 206 in the text file 200 provide a “mask” or structural model for identifying junctional epitopes. For example the first motif 206a shown in FIG. 11, XXXX(FY)XX(LIMV), defines an epitope that is eight amino acids in length. The value “X” indicates that any amino acid may be at that position of the epitope. The value “(FY)” indicates that either an F amino acid or a Y amino acid may be in the fifth position of the epitope. Similarly, “(LIMV)” indicates that any one of the listed amino acids, L, I, M or V, may be in the eighth position of the epitope. Therefore if a sequence of eight amino acids spanning a junction of two peptides satisfies the above motif criteria, it is identified as a junctional epitope.

FIG. 12 illustrates a flow chart diagram of one embodiment of the Junctional Analyzer program. At step 301, the program receives user inputs and instructions for performing the junctional analysis operation. In a preferred embodiment, the program uses an input text file 200 as shown in FIG. 11 to input parameters 202-226. As is well-known in the art, such a text file may be derived, for example, from a Microsoft Excel™ spreadsheet file or document, to specify desired input parameters (e.g., epitopes, motifs, flanking residue weight values, maximum number of hits, maximum search time, etc.) for its operation. At step 303, the Junctional Analyzer program generates a list of all epitope pairs. For example, if ten epitopes are entered by the user, there will be a total of ninety (10×9) epitope (peptide) pairs. Next, at step 305, for each pair of peptides or epitopes, the program determines the set of insertions that results in the minimum number of junctional epitopes and/or the maximum effect from the C+1 and N−1 contribution of spacing residues. To make this determination, the program calculates a function value for each possible combination of spacers for each peptide pair, where the number of spacers can range from 0 to MaxInsertions 208 (FIG. 11) and any arrangement of known or prespecified amino acids may be considered. In a preferred embodiment, the function value is calculated using the following equation: F=(C+N)/J, where C is the enhancement weight value for a flanking amino acid located at the C+1 position of an epitope, N is the enhancement weight value for a flanking amino acid located at the N−1 position of an epitope, and J is the number of junctional epitopes present. Since multiple motifs maybe satisfied at one junction of a peptide pair, J may be a number greater than one. When J=0, F=2(C+N). This second equation was chosen because for a fixed value of (C+N), the function value F will double when J changes from two to one, and will double again when J changes from one to zero. It is understood, however, that the above equations are exemplary only and that other equations for evaluating peptide pairs can be easily added to the program at any time. Modifications or changes to the above equations, depending on the desired criteria for emphasis or evaluation, would be readily apparent to those of ordinary skill in the art. At step 307, the program outputs the optimum combination of insertions (spacing and/or flanking residues) for each pair of peptides and the maximum function value for each pair of peptides. In a preferred embodiment, at step 307, the output from this program is generated as an output text file that lists, for each pair of peptides, the insertion that yields the maximum function result.

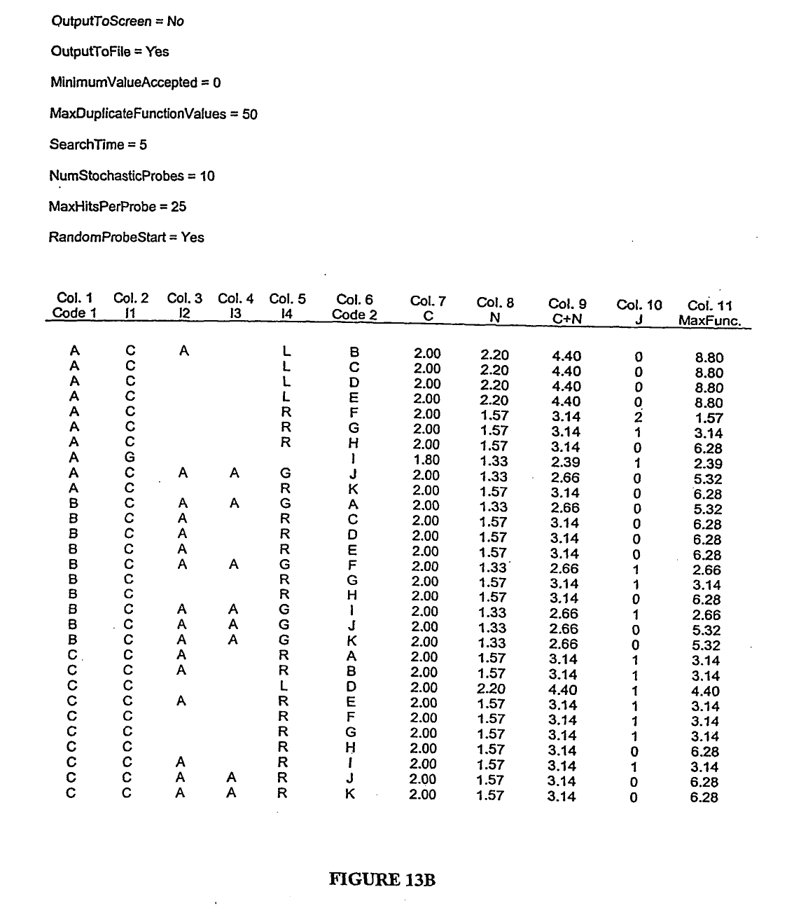

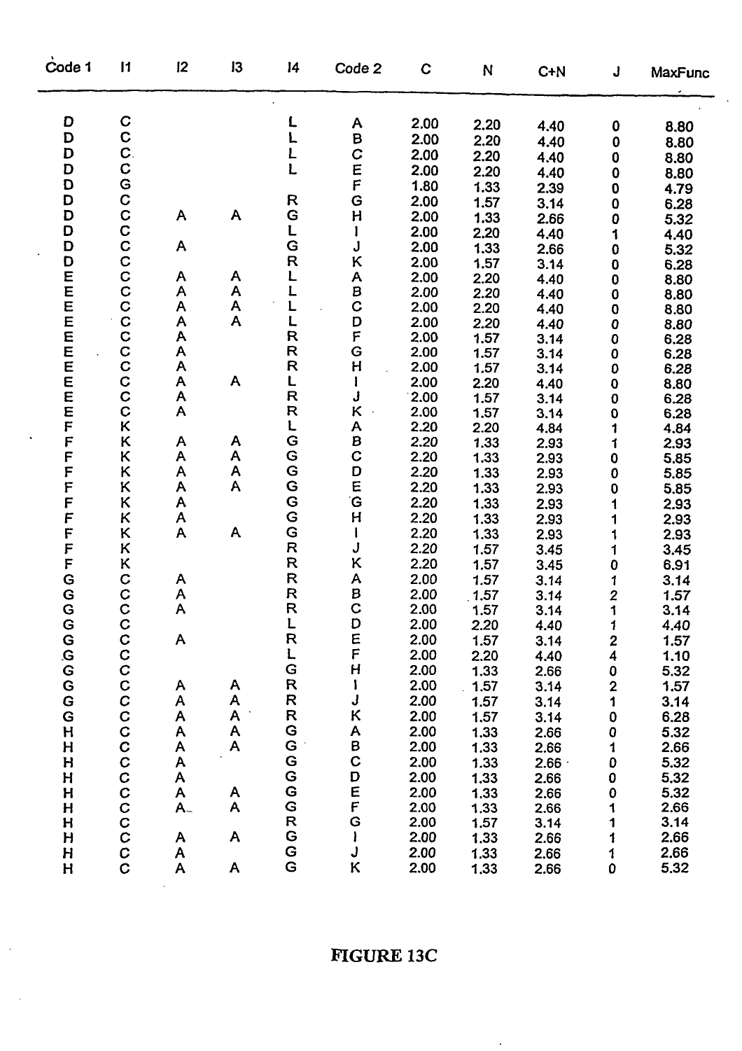

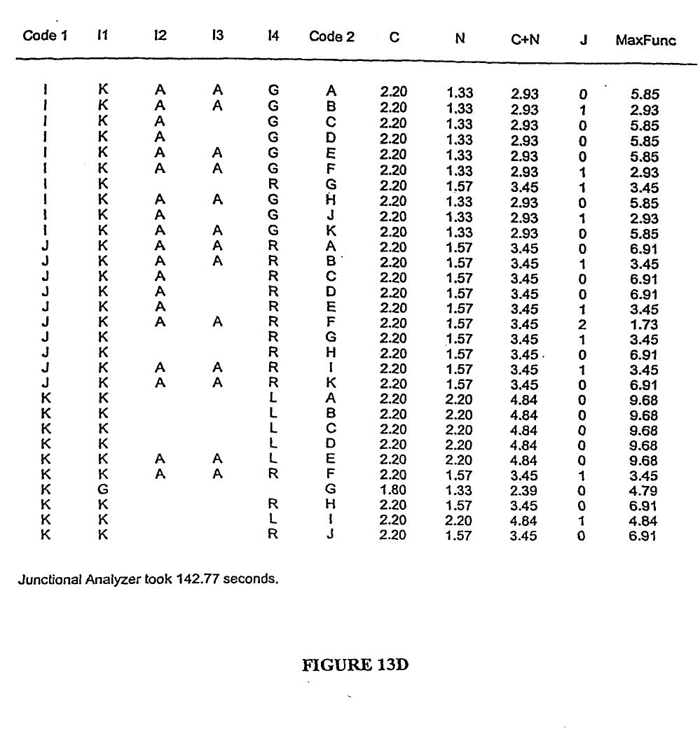

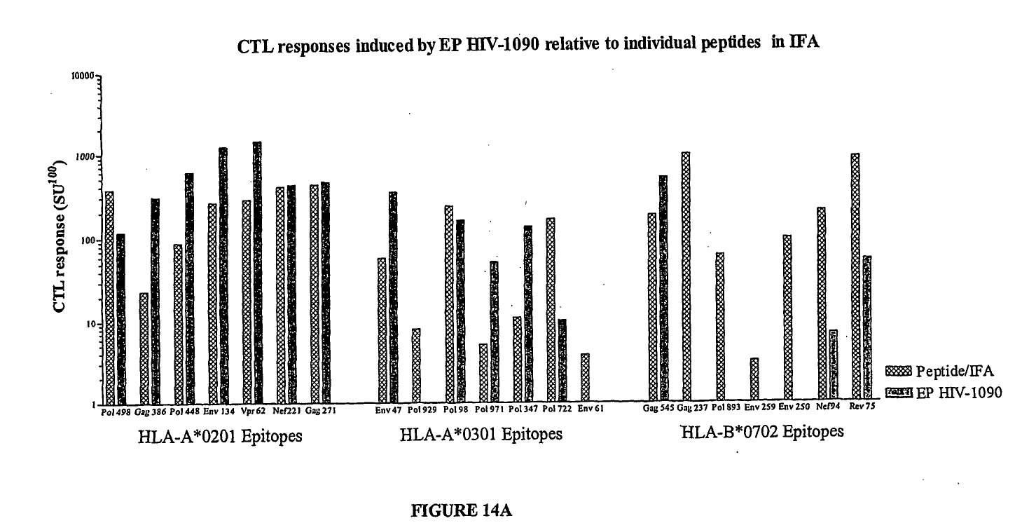

FIGS. 13A-D (hereinafter FIG. 13) illustrate an exemplary output text file 400 that lists, for each peptide pair, the spacer combination having the maximum function value. In the example shown in FIG. 13, eleven peptides, labeled A-K 202 (FIG. 11), were processed, the Motifs 206 were used to detect junctional epitopes, the enhancement weight values for each potential flanking residue 204 were used, and MaxInsertions 208 was set to four. Other parameters for controlling the operation and format of the Junctional Analyzer program were set as illustrated by the parameter settings 402. For purposes of convenience, in a preferred embodiment, these input parameters are repeated in the output text file 400. The output text file 400 includes an output table 404 which contain the results of steps 305 (FIG. 12). The first column (Col. 1) of the output table 404 indicates the first peptide of a pair. The second column (Col. 2) of the output table lists the first amino acid insertions which function both as a spacer and the C+1 flanking amino acid. The third column lists a second spacer amino acid. The fourth column lists a third spacer amino acid. The fifth column lists a fourth spacer amino acid which is also the N−1 flanking amino acid for the second peptide of the pair which is listed in column six. The seventh column lists the enhancement weight value of the C+1 flanking amino acid listed in column two. The eighth column lists the enhancement weight value of the N−1 flanking amino acid listed in column six 412. The ninth column lists the sum of the C+1 and N−1 enhancement weight values. The tenth column lists the number of junctional epitopes found in the peptide pair and the eleventh column lists the maximum function value for the peptide pair based on the equations listed above. For example, the first row of the output table 404 shows that for the peptide pair A-B, corresponding to the peptides VLAEAMSQV-ILKEPVHGV, the spacer combination of three amino acids, CAL, eliminates all junctional epitopes and provides a maximum function value of 8.80. It is understood, however, that other output options may be implemented in accordance with the invention. For example, the output table 404 may show the top 32 results for each pair of peptides, or show every result for all possible insertions in the order evaluated, or trace the motif search process to generate large output files, depending on the level of detail and/or analysis desired by the user.

In a preferred embodiment, the information contained in the output table 404 is used to perform either an “Exhaustive J Search” or a “Stochastic J Search” to identify a polypeptide construct linking all eleven peptides, including optimum spacer combinations. For eleven peptides, for example, there will be ten junctions. Therefore the permutation which yields the largest sum of function values taking into account all ten junctions is identified as the “optimum” permutation(s) of the multi-epitope constructs. In one embodiment, for the convenience of the user, the output text file 400 will also contain the original list of peptides/epitopes 202, the weight values used 204, the motifs used 206, and MaxInsertion value 208, and other parameter settings 402 entered into the input text file 200 of FIG. 11.

The “Exhaustive J Search” looks at all permutations of the peptides and selects the ones that have the largest function sum. However, due to the factorial nature of permutations, as the number of peptides to be processed increases, the time required to complete an Exhaustive J Search increases almost exponentially. For example, using a standard Macintosh 333 MHz computer, the estimated running time for 13 peptides is approximately 2.9 hours and would be approximately 40 hours for 14 peptides. The “Stochastic J Search” is designed to search many areas of the permutation sequence, rather than the entire permutation space, and report the best function sum that it finds. By reporting only permutations that meet or exceed the current maximum function total, it is possible to search a much broader area of the permutation sequence. This technique has been successful with as many as 20 peptides. The time to perform an exhaustive search of 20 peptides is estimated to be on the order of 1.3×105 years.

Referring again to FIG. 12, at step 309, the program determines whether to perform an Exhaustive or Stochastic search of the possible permutations of polypeptides from the output text file 400. In a preferred embodiment, the determination at step 309 is made by the user who inputs whether the search will be Exhaustive or Stochastic as indicated by the input parameter, Exhaustive (Y/N) 220 (FIG. 11). In other embodiments, the program may automatically select either a Stochastic or Exhaustive search depending on the number of peptides to be processed. For example, if less than 14 epitopes are to be included, an Exhaustive search routine is automatically selected by the program. The Exhaustive search program examines all permutations of the epitopes making up the multi-epitope construct to find the one(s) with the best value for the sum of the optimizing function for all pairs of epitopes. This is guaranteed to find the “best” permutation(s) since all are examined. If 14 or more epitopes are to be included in the multi-epitope construct, a Stochastic search is used. In a preferred embodiment, the Stochastic search uses a Monte Carlo technique, known to those of skill in the art, to examine many regions of the permutation space to find the best estimate of the optimum arrangement of the peptides. However, other methods of Stochastic searching may be implemented in accordance with the invention. For example, rather than randomly picking a starting permutation for each stochastic probe, the program may require that each probe begin with a permutation beginning with a different one of the peptides entered by the user. For example, if there were just three peptides, A, B and C, the three probes would begin with, for example, ABC, BAC and CBA. This method provides a fairly uniform coverage of the possible permutations.

If a Stochastic search has been selected, next, at step 311, the program begins the Stochastic search by initiating a probe. Next, at step 313, the program determines if the maximum search time per probe has been exceeded. If the maximum search time has not been reached, next, at step 315, the program determines whether a single probe has reached or exceeded the maximum number of “hits” per probe. In one embodiment, a probe hit is registered when a permutation's function value sum is the same as or greater than the largest function sum previously registered for one or more previously analyzed permutations. If the maximum number of hits per probe has not been reached, then, at step 317, the current stochastic probe evaluates the next permutation or set of permutations and the process returns step 313. If at step 315 it is determined that the maximum number of hits per probe has been reached or exceeded, then, the program proceeds to step 319, where the program determines whether a maximum number of probes have already been executed. Also, if at step 313, it is determined that the maximum time limit per probe has been reached or exceeded, the program proceeds to step 319 to determine if the maximum number of probes have been completed. If, at step 319, it is determined that the maximum number of probes has not been reached, the program returns to step 311 and a new search probe is initiated. If at step 319 it is determined that the maximum number of probes have been executed, the program then proceeds to step 323 where it outputs the best set of optimum permutations identified up to that point. This “best set” may consist of only those permutations having the highest function sum or, alternatively, may consist of the permutations having the top three highest function sums, for example, or any other output criteria desired by the user.

In one preferred embodiment, if a probe has received a maximum number of hits specified per probe, any unused time for that probe is divided by the remaining probes to decide how much time should be allocated to each of the remaining probes. In other words, if a probe terminates early because of finding too many hits then the remaining probes are allocated more time. Such functionality is easily implemented by those of ordinary skill in the computer programming arts.

If at step 309, an Exhaustive search has been selected, then, at step 321, an exhaustive search is initiated which analyzes every permutation, as described above. At the completion of the Exhaustive analysis, the program proceeds to step 323 where it outputs the “best set” of optimum permutations found. As mentioned above, this “best set” may include those permutations with the highest sum function values, or the top three highest sum function values, or permutations meeting any desired criteria specified by the user (e.g., top 30 permutations with the highest function values).

For each of the decision steps or determination steps discussed above (e.g., steps 313, 315 and 319), the program maybe set to perform a query at periodic intervals (e.g., every five seconds) or, alternatively, the program may be set to perform a query after a specified number of permutations (e.g., five) have been analyzed or after every permutation has been analyzed. Any one of these operation and timing protocols is easily implemented and adjusted by those of ordinary skill in the art.

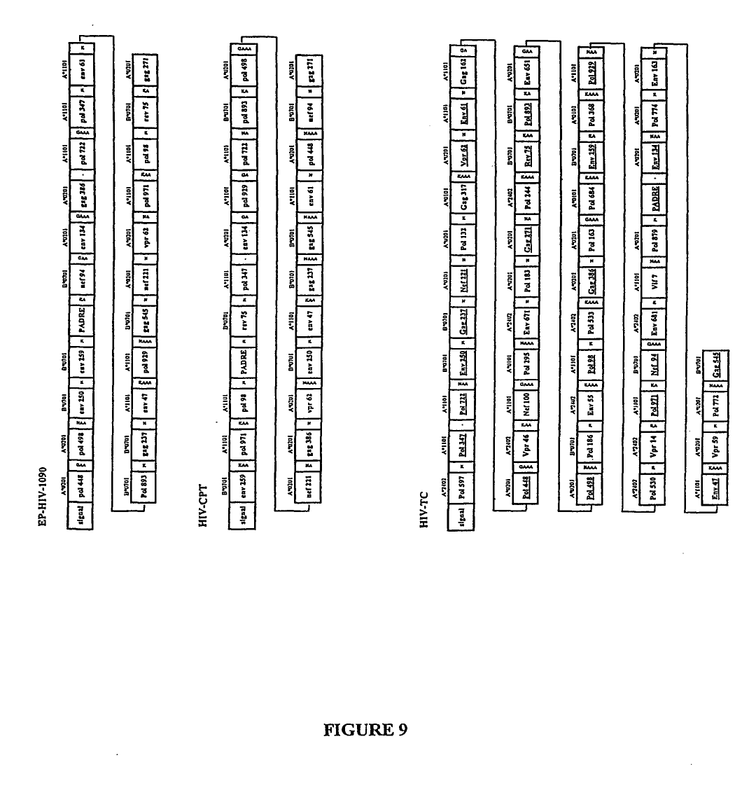

The Program output provides a list of the best arrangements of the epitopes. Since many permutations may have the same value of the evaluation function, several are generated so that other factors can be considered in choosing the optimum arrangement. Examples of multi-epitope constructs generated using the above-described computerized techniques are illustrated in FIG. 9. An exemplary process flow implemented by the method and system of the invention is provided above. As would be readily apparent to those of ordinary skill, other factors such as charge distribution, hydrophobic/hydrophilic region analysis, or folding prediction could also be incorporated into the evaluation function to further optimize the multi-epitope constructs. In addition, the multi-epitope construct may be further optimized by processing a multi-epitope construct already optimized by the process through the same or similar process one or more additional times. In the subsequent rounds of processing one or more parameters may be modified as compared to the parameters used in the first round of optimization. An example of a multi-epitope construct that was optimized in two rounds is the HBV-30CL construct.

Multi-epitope constructs can also be optimized by determining the structure of each construct to be considered. Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units.

Structural predictions such as charge distribution, hydrophobic/hydrophilic region analysis, or folding predictions can be performed using sequence analysis programs known to those of skill in the art, for example, hydrophobic and hydrophilic domains can be identified (see, e.g., Kyte & Doolittle, J. Mol. Biol. 157:105-132 (1982) and Stryer, Biochemistry (3^{rd} ed. 1988); see also any of a number of Internet based sequence analysis programs, such as those found at dot.imgen.bcm.tmc.edu.

A three-dimensional structural model of a multi-epitope construct can also be generated. This is generally performed by entering amino acid sequence to be analyzed into the computer system. The amino acid sequence represents the primary sequence or subsequence of the protein, which encodes the structural information of the protein. The three-dimensional structural model of the protein is then generated by the interaction of the computer system, using software known to those of skill in the art.

The amino acid sequence represents a primary structure that encodes the information necessary to form the secondary, tertiary and quaternary structure of the protein of interest. The software looks at certain parameters encoded by the primary sequence to generate the structural model. These parameters are referred to as “energy terms,” and primarily include electrostatic potentials, hydrophobic potentials, solvent accessible surfaces, and hydrogen bonding. Secondary energy terms include van der Waals potentials. Biological molecules form the structures that minimize the energy terms in a cumulative fashion. The computer program is therefore using these terms encoded by the primary structure or amino acid sequence to create the secondary structural model. The tertiary structure of the protein encoded by the secondary structure is then formed on the basis of the energy terms of the secondary structure. The user can enter additional variables such as whether the protein is membrane bound or soluble, its location in the body, and its cellular location, e.g., cytoplasmic, surface, or nuclear. These variables along with the energy terms of the secondary structure are used to form the model of the tertiary structure. In modeling the tertiary structure, the computer program matches hydrophobic faces of secondary structure with like, and hydrophilic faces of secondary structure with like. Those multi-epitope constructs that are most readily accessible to the HLA processing apparatus are then selected.

Assessment of Immunogenicity of Multi-Epitope Vaccines

The development of multi-epitope constructs represents a unique challenge, because the species-specificity of the peptide binding to MHC. Different MHC types from different species tend to bind different sets of peptides (Rammensee et al., Immunogenetics, Vol. 41(4):178-228 (1995)). As a result, it is not possible to test in regular laboratory animals a construct composed of human epitopes. Alternatives to overcome this limitation are generally available. They include: 1) testing analogous constructs incorporating epitopes restricted by non-human MHC; 2) reliance on control epitopes restricted by non human MHC; 3) reliance on crossreactivity between human and non-human MHC; 4) the use of HLA transgenic animals; and 5) antigenicity assays utilizing human cells in vivo. The following is a brief overview of the development of the technology for analyzing antigenicity and immunogenicity.

Class I HLA Transgenics

The utility of HLA transgenic mice for the purpose of epitope identification (Sette et al., J Immunol, Vol. 153(12):5586-92 (1994); Wentworth et al., Int Immunol, Vol. 8(5):651-9 (1996); Engelhard et al., J Immunol, Vol. 146(4):1226-32 (1991); Man et al., Int Immunol, Vol. 7(4):597-605 (1995); Shirai et al., J Immunol, Vol. 154(6):2733-42 (1995)), and vaccine development (Ishioka et al., J Immunol, Vol. 162(7):3915-25 (1999)) has been established. Most of the published reports have investigated the use of HLA A2.1K^{b} mice but it should be noted that B*27, and B*3501 mice are also available. Furthermore, HLA A*11/K^{b} mice (Alexander et al., J Immunol, Vol. 159(10):4753-61 (1997)), and HLA B7/K^{b} and HLA A1/K^{b} mice have also been generated.

Data from 38 different potential epitopes was analyzed to determine the level of overlap between the A2.1-restricted CTL repertoire of A2.1/K^{b}-transgenic mice and A2.1+humans (Wentworth et al., Eur J Immunol, Vol. 26(1):97-101 (1996)). In both humans and mice, an MHC peptide binding affinity threshold of approximately 500 nM correlates with the capacity of a peptide to elicit a CTL response in vivo. A high level of concordance between the human data in vivo and mouse data in vivo was observed for 85% of the high-binding peptides, 58% of the intermediate binders, and 83% of the low/negative binders. Similar results were also obtained with HLA A11 and HLA B7 transgenic mice (Alexander et al., J Immunol, Vol. 159(10):4753-61 (1997)). Thus, because of the extensive overlap that exists between T cell receptor repertoires of HLA transgenic mouse and human CTLs, transgenic mice are valuable for assessing immunogenicity of the multi-epitope constructs described herein.

The different specificities of TAP transport as it relates to HLA A11 mice does not prevent the use of HLA-A11 transgenic mice of evaluation of immunogenicity. While both murine and human TAP efficiently transport peptides with an hydrophobic end, only human TAP has been reported to efficiently transport peptides with positively charged C terminal ends, such as the ones bound by A3, A11 and other members of the A3 supertype. This concern does not apply to A2, A1 or B7 because both murine and human TAP should be equally capable of transporting peptides bound by A2, B7 or A1 . Consistent with this understanding, Vitiello (Vitiello et al., J Exp Med, Vol. 173(4):1007-15 (1991)) and Rotzschke (Rotzschke O, Falk K., Curr Opin Immunol, Vol. 6(1):45-51 (1994)) suggested that processing is similar in mouse and human cells, while Cerundolo (Rotzschke O, Falk K., Curr Opin Immunol, Vol. 6(1):45-51 (1994)) suggested differences in murine versus human cells, both expressing HLA A3 molecules. However, using HLA A11 transgenics, expression of HLA molecules on T and B cells in vivo has been observed, suggesting that the reported unfavorable specificity of murine TAP did not prevent stabilization and transport of A11/K^{b} molecules in vivo (Alexander et al., J Immunol, Vol. 159(10):4753-61 (1997)). These data are in agreement with the previous observation that peptides with a charged C termini could be eluted from murine cells transfected with A11 molecules (Maier et al., Immunogenetics; Vol. 40(4):306-8 (1994)). Responses in HLA A11 mice to complex antigens, such as influenza, and most importantly to A11 restricted epitopes encoded by multi-epitope constructs (Ishioka et al., J Immunol, Vol. 162(7):3915-25 (1999)) has also been detected. Thus, the TAP issue appears to be of minor concern with transgenic mice.

Another issue of potential relevance in the use of HLA transgenic mice is the possible influence of β2 microglobulin on HLA expression and binding specificity. It is well known that human β2 binds both human and mouse MHC with higher affinity and stability than mouse β2 microglobulin (Shields et al., Mol Immunol Vol. 35(14-15):919-28 (1998)). It is also well known that more stable complexes of MHC heavy chain and β2 facilitate exogenous loading of MHC Class I (Vitiello et al., Science, Vol. 250(4986):1423-6 (1990)). We have examined the potential effect of this variable by generating mice that are double transgenics for HLA/K^{b} and human β2. Expression of human β2 was beneficial in the case HLA B7/K^{b} mice, and was absolutely essential to achieve good expression levels in the case of HLA A1 transgenic mice. Accordingly, HLA/K^{b} and β2 double transgenic mice are currently and routinely bred and utilized by the present inventors. Thus, HLA transgenic mice can be used to model HLA-restricted recognition of four major HLA specificities (namely A2, A11, B7 and A1) and transgenic mice for other HLA specificities can be developed as suitable models for evaluation of immunogenicity.

Antigenicity Testing for Class I Epitopes

Several independent lines of experimentation indicate that the density of Class I/peptide complexes on the cell surface may correlate with the level of T cell priming. Thus, measuring the levels at which an epitope is generated and presented on an APC's surface provides an avenue to indirectly evaluate the potency of multi-epitope nucleic acid vaccines in human cells in vitro. As a complement to the use of HLA Class I transgenic mice, this approach has the advantage of examining processing in human cells. (Ishioka et al., J Immunol, Vol. 162(7):3915-25 (1999))

Several possible approaches to experimentally quantitate processed peptides are available. The amount of peptide on the cell surface can be quantitated by measuring the amount of peptide eluted from the APC surface (Sijts et al., J Immunol, Vol. 156(2):683-92 (1996); Demotz et al., Nature, Vol. 342(6250):682-4 (1989)). Alternatively, the number of peptide-MHC complexes can be estimated by measuring the amount of lysis or lymphokine release induced by infected or transfected target cells, and then determining the concentration of peptide necessary to obtain equivalent levels of lysis or lymphokine release (Kageyama et al., J Immunol, Vol. 154(2):567-76 (1995)).

A similar approach has also been used to measure epitope presentation in multi-epitope nucleic acid-transfected cell lines. Specifically, multi-epitope constructs that are immunogenic in HLA transgenic mice are also processed into optimal epitopes by human cells transfected with the same constructs, and the magnitude of the response observed in transgenic mice correlates with the antigenicity observed with the transfected human target cells (Ishioka et al., J Immunol, Vol. 162(7):3915-25 (1999)).

Using antigenicity assays, a number of related constructs differing in epitope order or flanking residues can be transfected into APCs, and the impact of the aforementioned variables on epitope presentation can be evaluated. This can be a preferred system for testing where a relatively large number of different constructs need to be evaluated. Although it requires large numbers of epitope-specific CTLs, protocols that allow for the generation of highly sensitive CTL lines (Alexander-Miller et al., Proc Natl Acad Sci US A, Vol. 93(9):4102-7 (1996)) and also for their expansion to large numbers (Greenberg P. D., Riddell S. R., Science, Vol. 285(5427):546-51 (1999)) have been developed to address this potential problem.

It should also be kept in mind that, if the cell selected for the transfection is not reflective of the cell performing APC function in vivo, misleading results could be obtained. Cells of the B cell lineage, which are known “professional” APCs, are typically employed as transfection recipients. The use of transfected B cells of this type is an accepted practice in the field. Furthermore, a good correlation has already been noted between in vitro data utilizing transfected human B cells and in vivo results utilizing HLA transgenic mice, as described in more detail herein.

Measuring HTL Responses

In preferred embodiments, vaccine constructs are optimized to induce Class II restricted immune responses. One method of evaluating multi-epitope constructs including Class II epitopes, is to use HLA-DR transgenic mice. Several groups have produced and characterized HLA-DR transgenic mice (Taneja V., David C. S., Immunol Rev, Vol. 169:67-79 (1999)).

An alternative also exists which relies on crossreactivity between certain human MHC molecules and particular MHC molecules expressed by laboratory animals. Bertoni and colleagues (Bertoni et al., J Immunol, Vol. 161(8):4447-55 (1998)) have noted that appreciable crossreactivity can be demonstrated between certain HLA Class I supertypes and certain PATR molecules expressed by chimpanzees. Crossreactivity between human and macaques at the level of Class II (Geluk et al., J Exp Med, Vol. 177(4):979-87 (1993)) and Class I molecules (Dzuris, et al., J. Immunol., July 1999) has also been noted. Finally, it can also be noted that the motif recognized by human HLA B7 supertype is essentially the same as the one recognized by the murine Class I L^{d} (Rammensee et al., Immunogenetics, Vol. 41(4):178-228 (1995)). Of relevance to testing HLA DR restricted epitopes in mice, it has been shown by Wall, et al (Wall et al., J. Immunol., 152:4526-36 (1994)) that similarities exist in the motif of DR1 and IA^{b}. We routinely breed our transgenic mice to take advantage of this fortuitous similarity. Furthermore, we have also shown that most of our peptides bind to IA^{b}, so that we use these mice for the study of CTL and HTL immunogenicity.

Measuring and Quantitating Immune Responses from Clinical Samples

A crucial element to assess vaccine performance is to evaluate its capacity to induce immune responses in vivo. Analyses of CTL and HTL responses against the immunogen, as well as against common recall antigens are commonly used and are known in the art. Assays employed included chromium release, lymphokine secretion and lymphoproliferation assays.

More sensitive techniques such as the ELISPOT assay, intracellular cytoline staining, and tetramer staining have become available in the art. It is estimated that these newer methods are 10- to 100-fold more sensitive than the common CTL and HTL assays (Murali-Krishna et al., Immunity, Vol. 8(2): 177-87 (1998)), because the traditional methods measure only the subset of T cells that can proliferate in vitro, and may, in fact, be representative of only a fraction of the memory T cell compartment (Ogg G. S., McMichael A. J., Curr Opin Immunol, Vol. 10(4):393-6 (1998)). Specifically in the case of HIV, these techniques have been used to measure antigen-specific CTL responses from patients that would have been undetectable with previous techniques (Ogg et al., Science, Vol. 279(5359):2103-6 (1998); Gray et al., J Immunol, Vol. 162(3):1780-8 (1999); Ogg et al., J Virol, Vol. 73(11):9153-60 (1999); Kalams et al., J Virol, Vol. 73(8):6721-8 (1999); Larsson et al., AIDS, Vol. 13(7):767-77 (1999); Come et al., J Acquir Immune Defic Syndr Hum Retrovirol, Vol. 20(5):442-7 (1999)).

With relatively few exceptions, direct activity of freshly isolated cells has been difficult to demonstrate by the means of traditional assays (Ogg G. S., McMichael A. J., Curr Opin Immunol, Vol. 10(4):393-6 (1998)). However, the increased sensitivity of the newer techniques has allowed investigators to detect responses from cells freshly isolated from infected humans or experimental animals (Murali-Krishna et al., Immunity, Vol. 8(2):177-87 (1998); Ogg G. S., McMichael A. J., Curr Opin Immunol, Vol. 10(4):393-6 (1998)). The availability of these sensitive assays, that do not depend on an in vitro restimulation step, has greatly facilitated the study of CTL function in natural infection and cancer. In contrast, assays utilized as an endpoint to judge effectiveness of experimental vaccines are usually performed in conjunction with one or more in vitro restimulation steps (Ogg G. S., McMichael A. J., Curr Opin Immunol, Vol. 10(4):393-6 (1998)). In fact, with few exceptions (Hanke et al., Vaccine, Vol. 16(4):426-35 (1998)), freshly isolated Class I-restricted CD8+ T cells have been difficult to demonstrate in response to immunization with experimental vaccines designed to elicit CTL responses. The use of sensitive assays, such as ELISPOT or in situ IFNγ ELISA, have been combined with a restimulation step to achieve maximum sensitivity; MHC tetramers are also used for this purpose.

MHC tetramers were first described in 1996 by Altman and colleagues. They produced soluble HLA-A2 Class I molecules which were folded with HIV-specific peptides containing a CTL epitope complexed together into tetramers tagged with fluorescent markers. These are used to label populations of T cells from HIV-infected individuals that recognize the epitope (Ogg G. S., McMichael A. J., Curr Opin Immunol, Vol. 10(4):393-6 (1998)). These cells were then quantified by flow cytometry, providing a frequency measurement for the T cells that are specific for the epitope. This technique has become very popular in HIV research as well as in other infectious diseases (Ogg G. S., McMichael A. J., Curr Opin Immunol, Vol. 10(4):393-6 (1998); Ogg et al., Science, Vol. 279(5359):2103-6 (1998); Gray et al., J Immunol, Vol. 162(3):1780-8 (1999); Ogg et al., J Virol, Vol. 73(11):9153-60 (1999); Kalams et al., J Virol, Vol. 73(8):6721-8 (1999)). However, HLA polymorphism can limit the general applicability of this technique, in that the tetramer technology relies on defined HLA/peptide combinations. However, it has been shown that a variety of peptides, including HIV-derived peptides, are recognized by peptide-specific CTL lines in the context of different members of the A2, A3 and B7 supertypes (Threlkeld et al., J Immunol, Vol. 159(4):1648-57 (1997); Bertoni et al., J Clin Invest, Vol. 100(3):503-13 (1997)). Taken together these observations demonstrate that a T cell receptor (TCR) for a given MHC/peptide combination can have detectable affinity for the same peptide presented by a different MHC molecule from the same supertype.

In circumstances in which efficacy of a prophylactic vaccine is primarily correlated with the induction of a long-lasting memory response, restimulation assays can be the most appropriate and sensitive measures to monitor vaccine-induced immunological responses. Conversely, in the case of therapeutic vaccines, the main immunological correlate of activity can be the induction of effector T cell function, most aptly measured by primary assays. Thus, the use of sensitive assays allows for the most appropriate testing strategy for immunological monitoring of vaccine efficacy.

Antigenicity of Multi-Epitope Constructs in Transfected Human APC's

Antigenicity assays are performed to evaluate epitope processing and presentation in human cells. An episomal vector to efficiently transfect human target cells with multi-epitope nucleic acid vaccines is used to perform such an analysis.

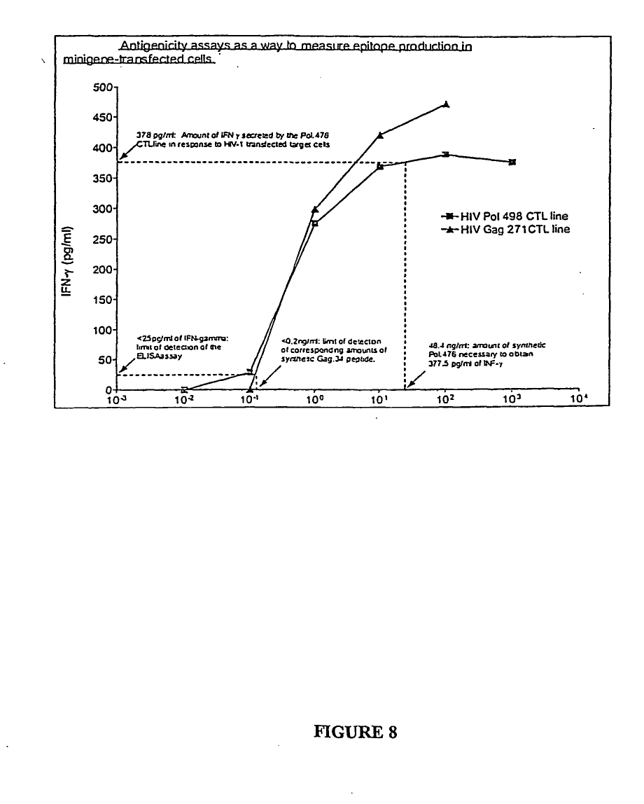

For example, 221 A2K^{b} target cells were transfected with an HIV-1 EpiGene™ vaccine. The 221 A2K^{b} target cell expresses the A2K^{b} gene that is expressed in HLA transgenic mice, but expresses no endogenous Class I (Shimizu Y, DeMars R., J Immunol, Vol. 142(9):3320-8 (1989)). These transfected cells were assayed for their capacity to present antigen to CTL lines derived from HLA transgenic mice and specific for various HIV-derived CTL epitopes. To correct for differences in antigen sensitivity of different CTL lines, peptide dose titrations, using untransfected cells as APC, were run in parallel. Representative data is presented in FIG. 8. In the case of HIV Pol 498-specific CTL, the transfected target cells induced the release of 378 pg/ml of IFNγ. Inspection of the peptide dose responses reveals that, 48 ng/ml of exogenously added peptide was necessary to achieve similar levels of IFNγ release. These results demonstrate that relatively large amounts of Pol 498 epitope are presented by the transfected cells, equivalent to 48 ng/ml of exogenously added peptide.

TABLE 5
Comparison between antigenicity in transfected human cells and
immunogenicity in HLA transgenic mice of the HIV-1 minigene

| | Antigenicity | | Immunogenicity | |
|---|---|---|---|---|
| Epitope | Peptide Equivalents¹⁾ | n²⁾ | % response³⁾ | Magnitude⁴⁾ |
| HIV Pol 498 | 30.5 | (6) | 95% | 46.7 |
| HIV Env 134 | 6.2 | (3) | 62% | 16.1 |
| HIV Nef 221 | 2.1 | (5) | 82% | 3.8 |
| HIV Gag 271 | <0.2 | (6) | 31% | 4 |

¹⁾ng/ml;
²⁾number of independent experiments;
³⁾% of CTL cultures yielding positive results;
⁴⁾Lytic Units

By comparison, less than 25 pg/ml IFNγ was detected utilizing the CTL specific for the Gag 271 epitope. The control peptide titration with untransfected target cells revealed that this negative result could not be ascribed to poor sensitivity of the particular CTL line utilized, because as little as 0.2 pg/ml of “peptide equivalents” (PE) could be detected. Thus, it appears that the Gag 271 epitope is not efficiently processed and presented in the HIV- 1 transfected target cells. Utilizing the “peptide equivalents” figure as an approximate quantitation of the efficiency of processing, it can be estimated that at least 200-fold less Gag 271 is presented by the transfected targets, compared to the Pol 498 epitope.

The results of various independent determinations for four different epitopes contained within HIV-FT are compiled in Table 5. The amount of each epitope produced from the HIV-FT transfected cells ranged from 30.5 PE for Pol 498, to a low of less than 0.2 PE for Gag 271. The two epitopes Env 134 and Nef221 were associated with intermediate values, of 6.1 and 2.1 PE, respectively.

These results were next correlated with the in vivo immunogenicity values observed for each epitope after immunization with the HIV-FT construct. The Pol 498 epitope was also the most immunogenic, as would be predicted. The Env 134 and Nef 221 epitopes, for which intermediate immunogenicity was observed in vivo, were also processed in vitro with intermediate efficiency by the transfected human cells. Finally, the Gag 271, for which no detectable in vitro processing was observed was also associated with in vivo immunogenicity suboptimal in both frequency and magnitude.

These data have several important implications. First, they suggest that different epitopes contained within a given construct may be processed and presented with differential efficiency. Second, they suggest that immunogenicity is proportional to the amount of processed epitope generated. Finally, these results provide an important validation of the use of transgenic mice for the purpose of optimization of multi-epitope vaccines destined for human use.

III. Preparation of Multi-Epitope Constructs

Epitopes for inclusion in the multi-epitope constructs typically bear HLA Class I or Class II binding motifs as described, for example, in PCT applications PCT/US00/27766, or PCT/US00/19774. Multi-epitope constructs can be prepared according to the methods set forth in Ishioka, et al., J Immunol (1999) 162(7):3915-3925, for example.

Multiple HLA class II or class I epitopes present in a multi-epitope construct can be derived from the same antigen, or from different antigens. For example, a multi-epitope construct can contain one or more HLA epitopes that can be derived from two different antigens of the same virus or from two different antigens of different viruses. Epitopes for inclusion in a multi-epitope construct can be selected by one of skill in the art, e.g., by using a computer to select epitopes that contain HLA allele-specific motifs or supermotifs. The multi-epitope constructs of the invention can also encode one or more broadly cross-reactive binding, or universal, HLA class II epitopes, e.g., PADRE® (Epimmune, San Diego, Calif.), (described, for example, in U.S. Pat. No. 5,736,142) or a PADRE® family molecule.

Universal HLA Class II epitopes can be advantageously combined with other HLA Class I and Class II epitopes to increase the number of cells that are activated in response to a given antigen and provide broader population coverage of HLA-reactive alleles. Thus, the multi-epitope constructs of the invention can include HLA epitopes specific for an antigen, universal HLA class II epitopes, or a combination of specific HLA epitopes and at least one universal HLA class II epitope.

HLA Class I epitopes are generally about 8 to about 13 amino acids in length, in particular 8, 9, 10, or 11 amino acids in length. HLA Class II epitopes are generally about 6 to 25 amino acids in length, in particular about 13 to 21 amino acids in length. An HLA Class I or II epitope can be derived from any desired antigen of interest. The antigen of interest can be a viral antigen, surface receptor, tumor antigen, oncogene, enzyme, or any pathogen, cell or molecule for which an immune response is desired. Epitopes can be selected based on their ability to bind one or multiple HLA alleles. Epitopes that are analogs of naturally occuring sequences can also be included in the multi-epitope constructs described herein. Such analog peptides are described, for example, in PCT applications PCT/US97/03778, PCT/US00/19774, and co-pending U.S. Ser. No. 09/260,714 filed Mar. 1, 1999.

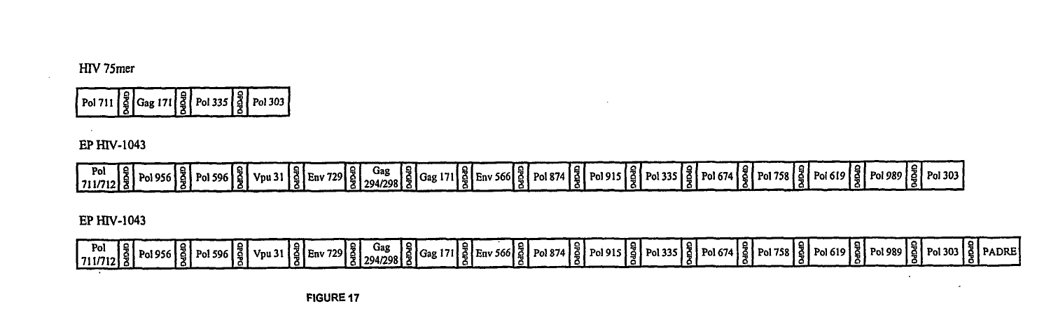

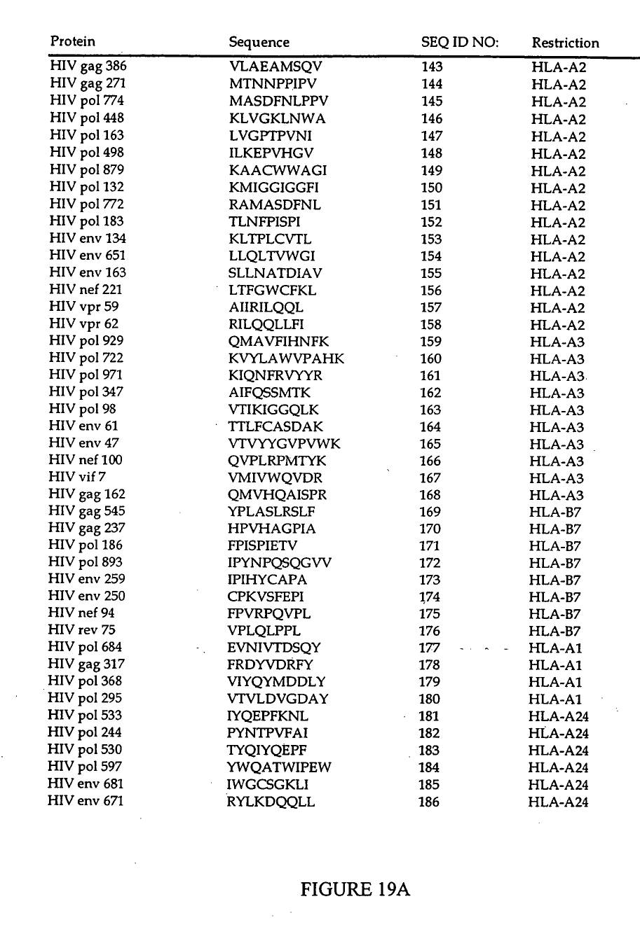

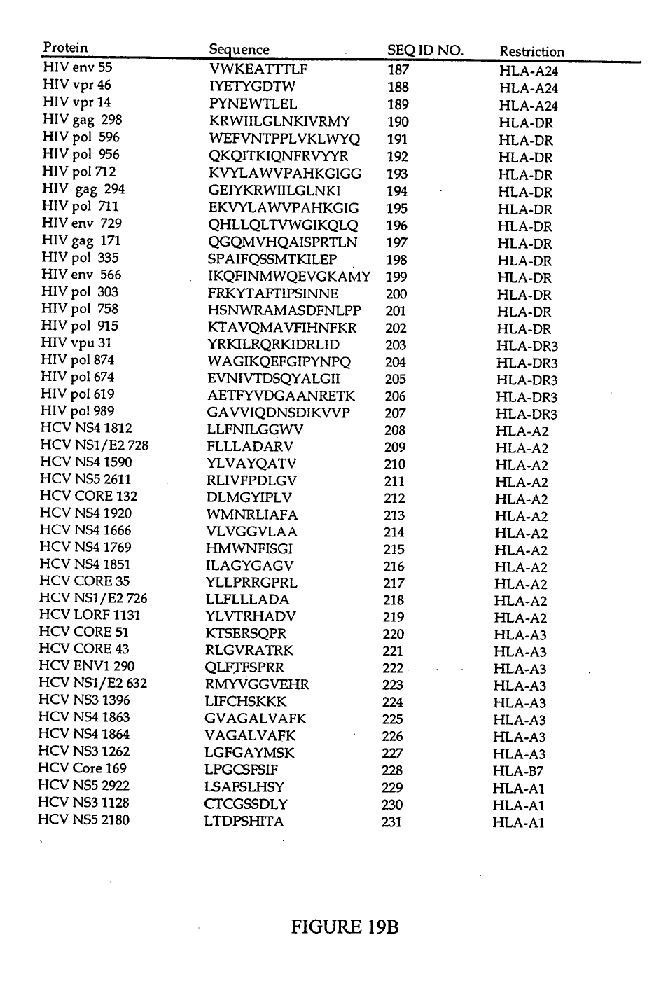

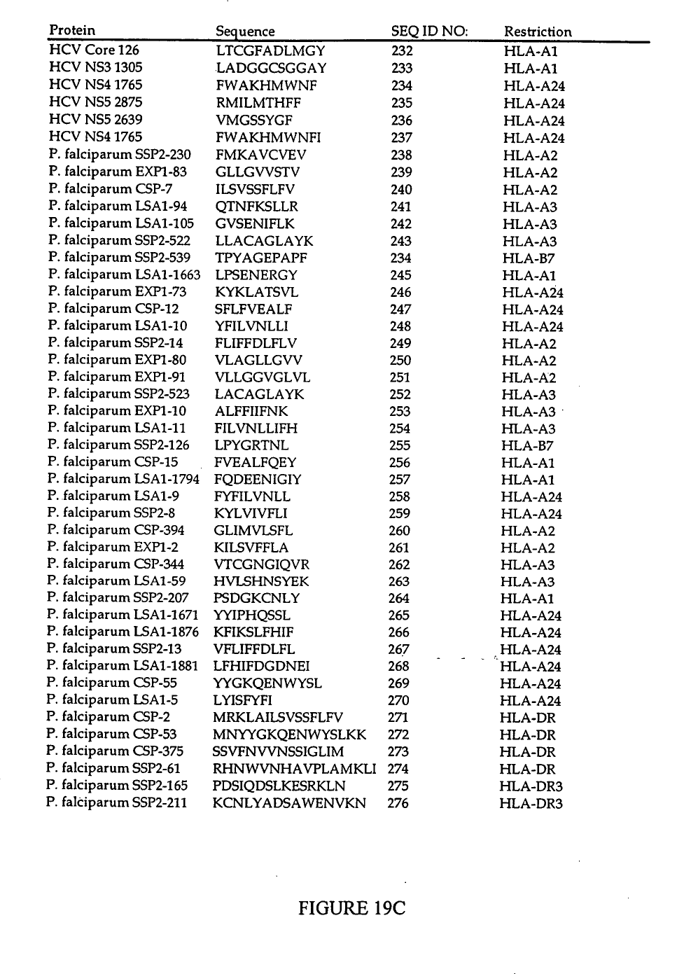

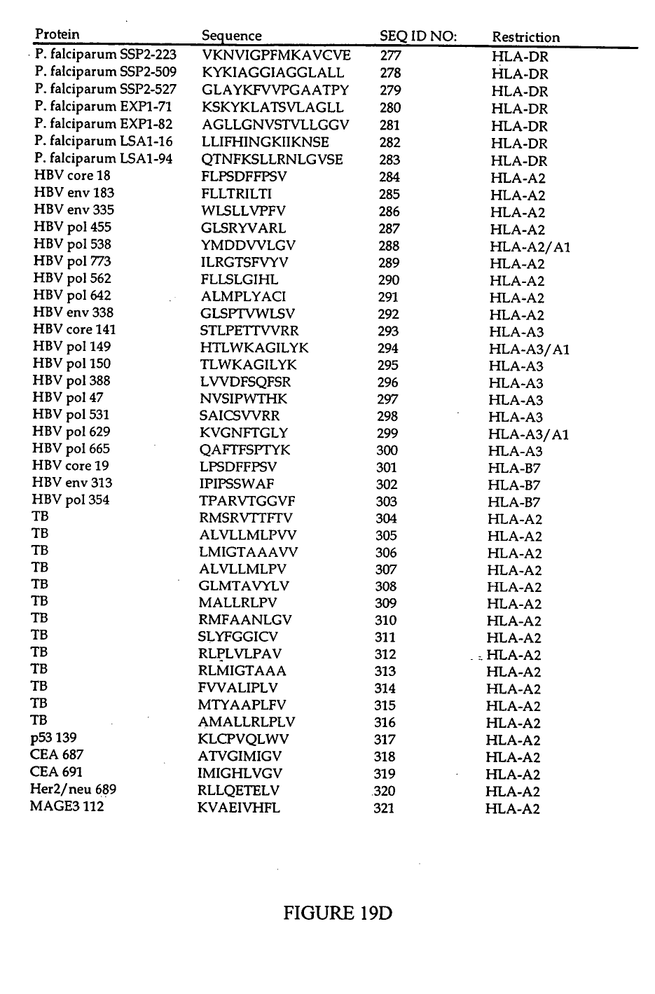

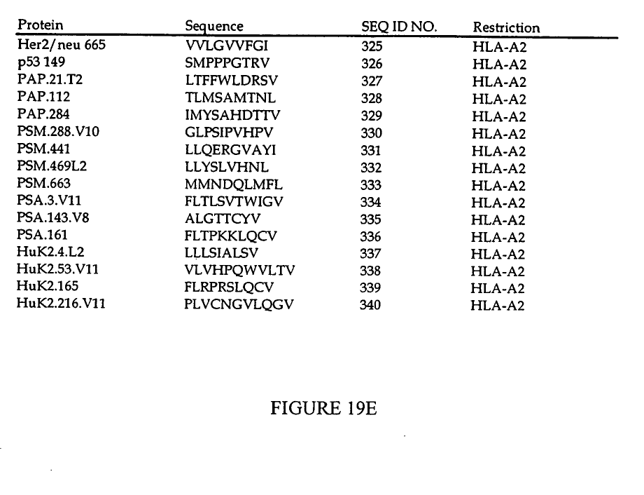

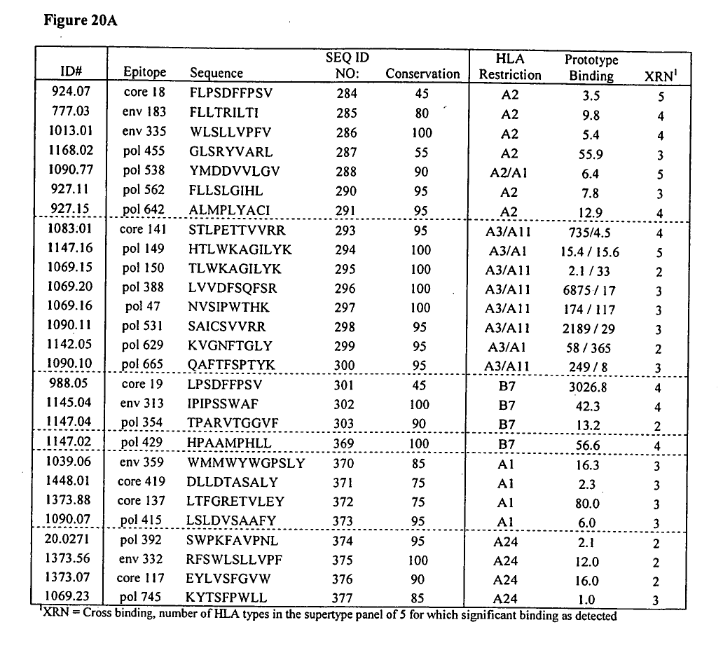

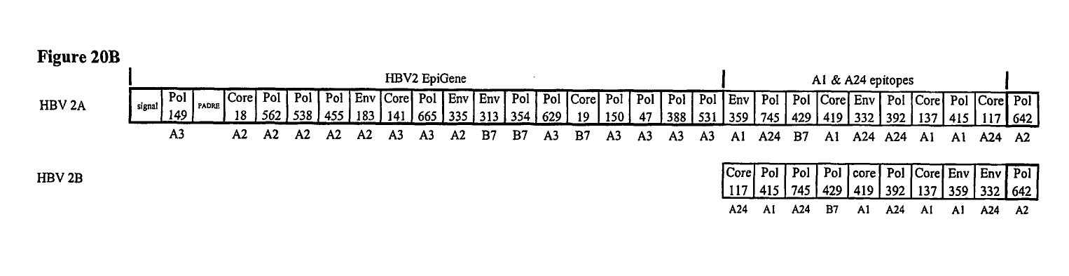

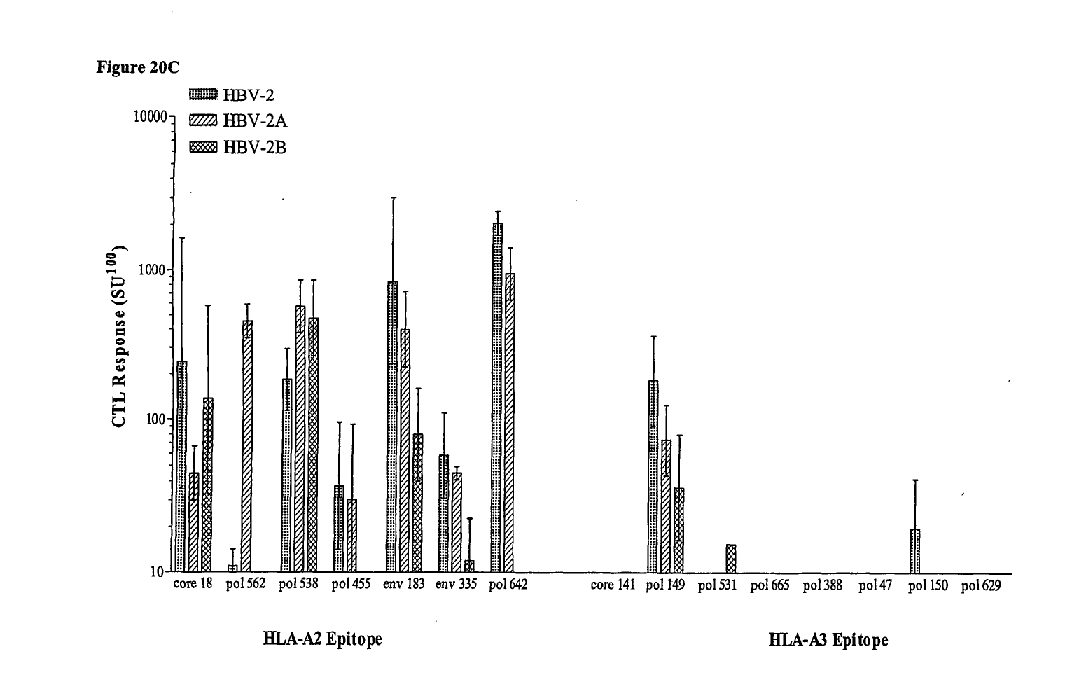

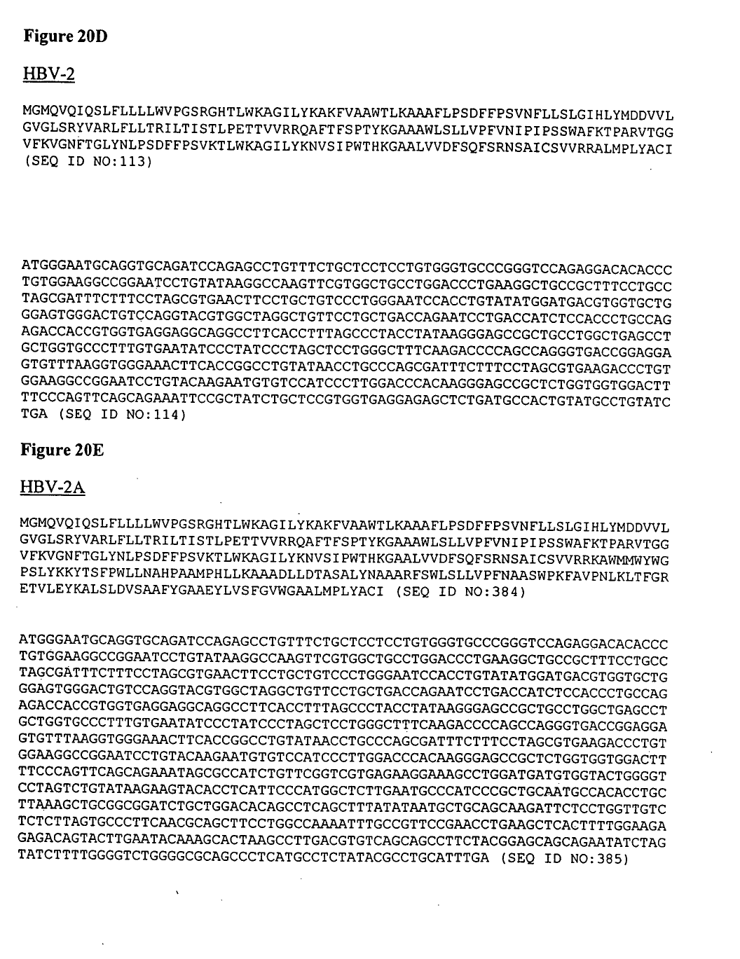

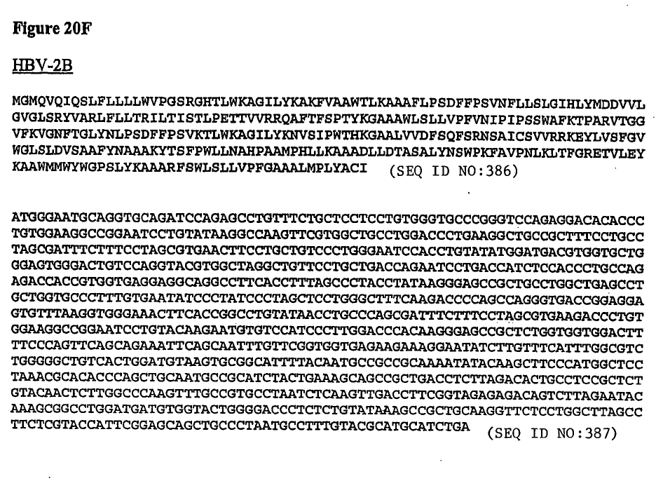

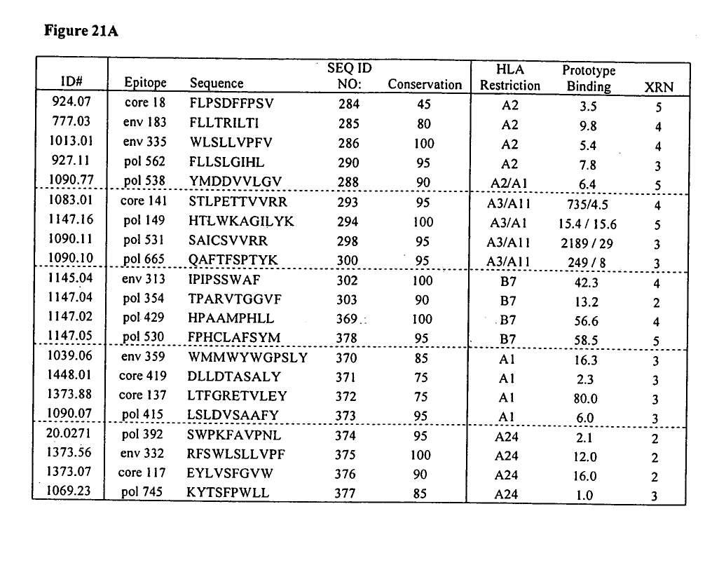

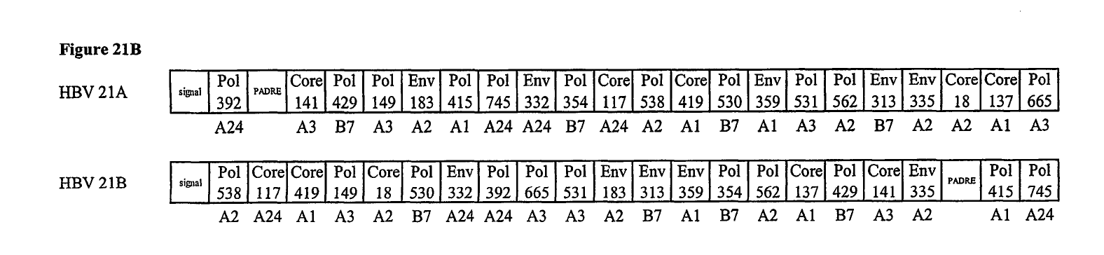

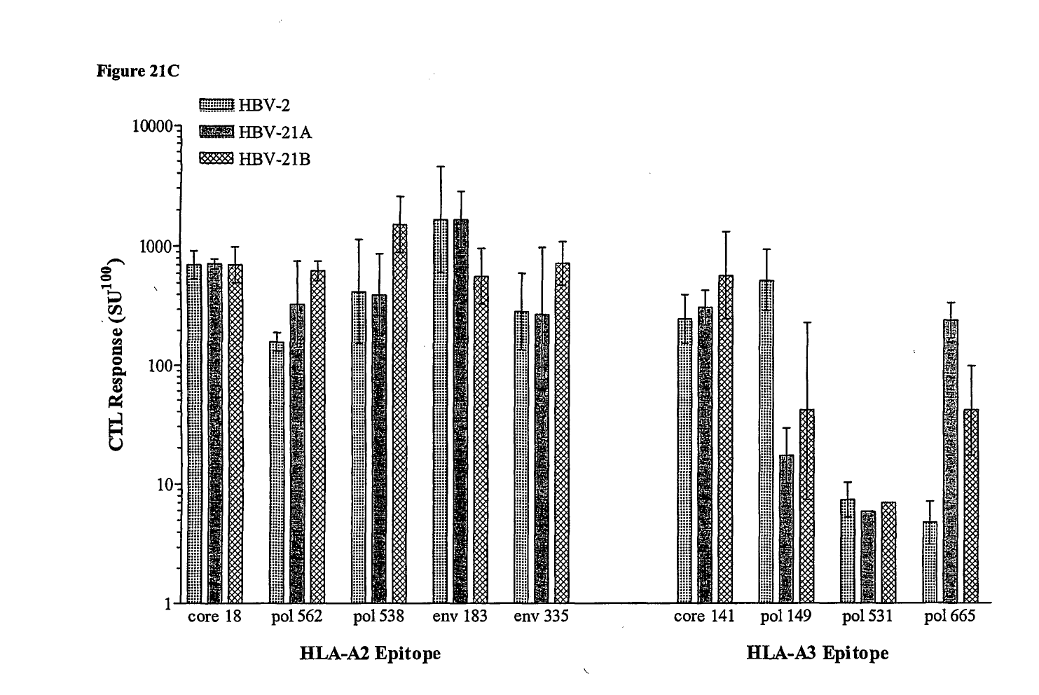

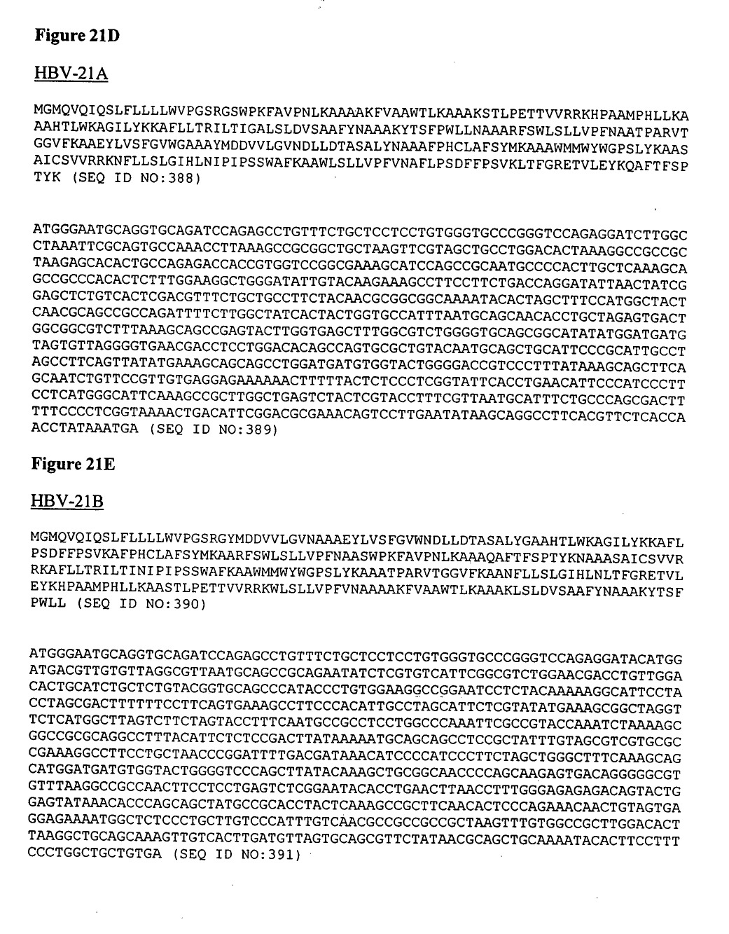

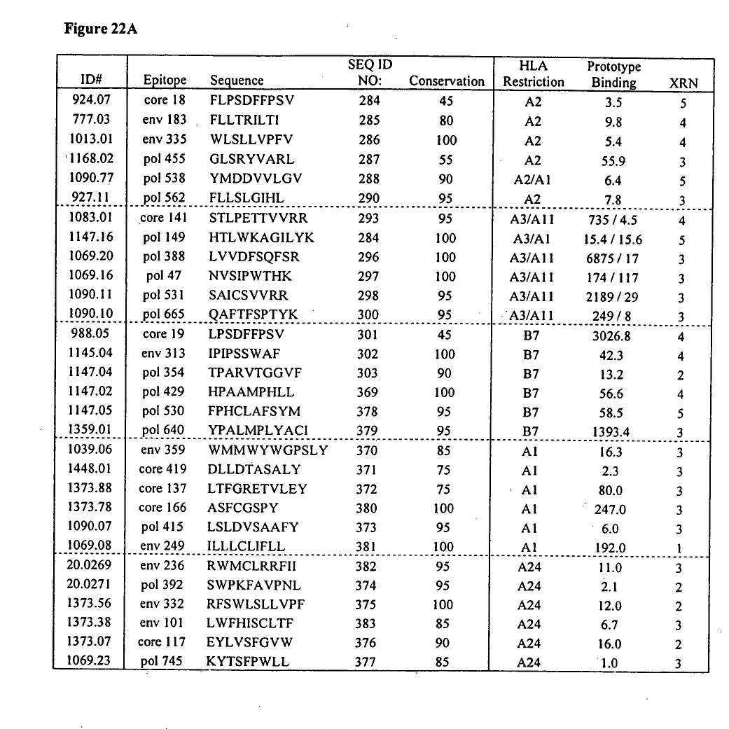

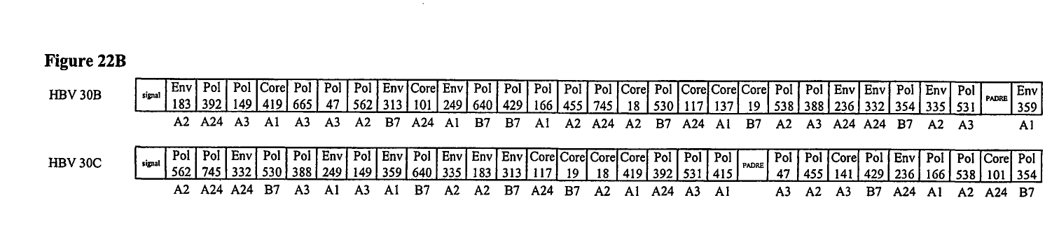

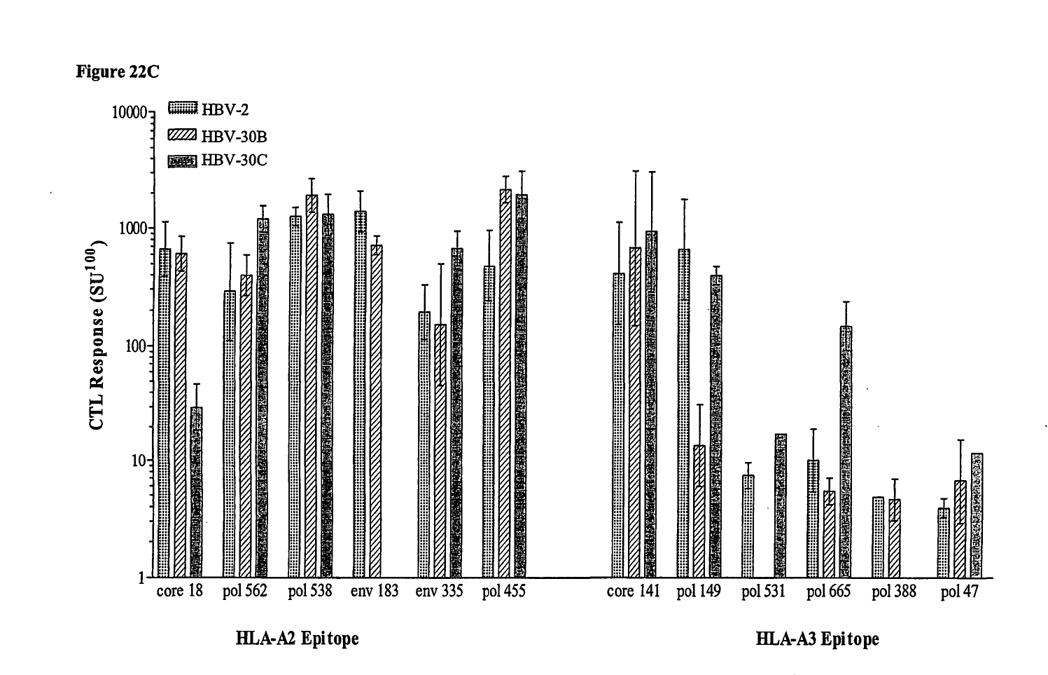

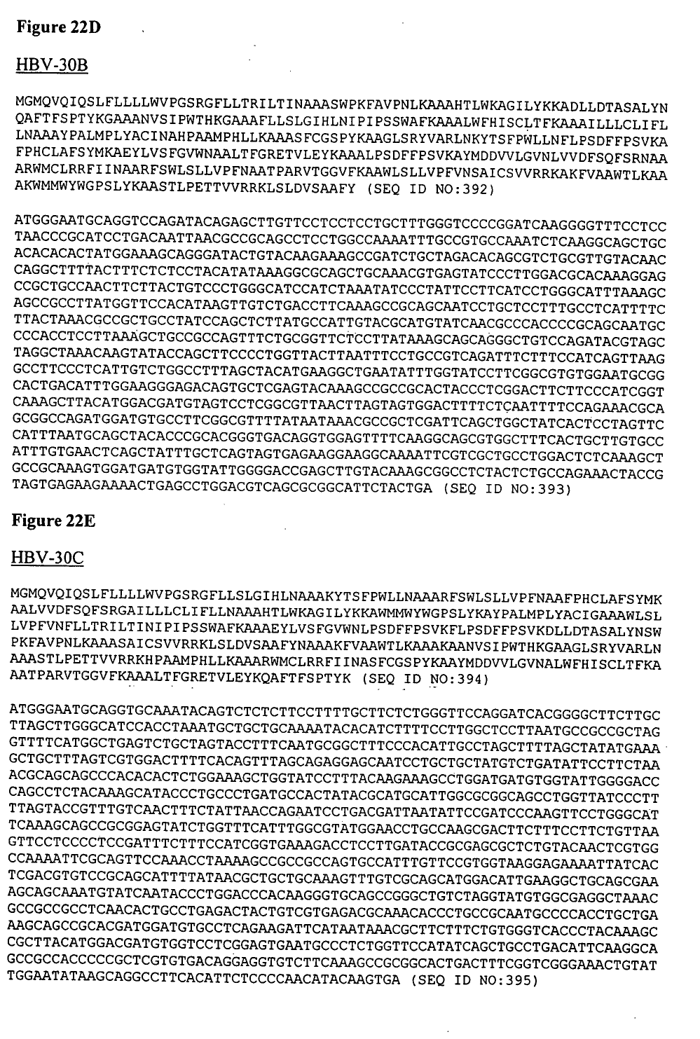

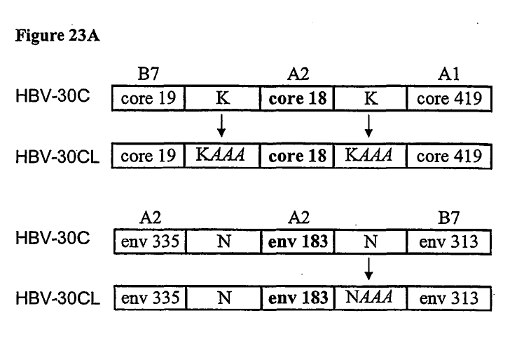

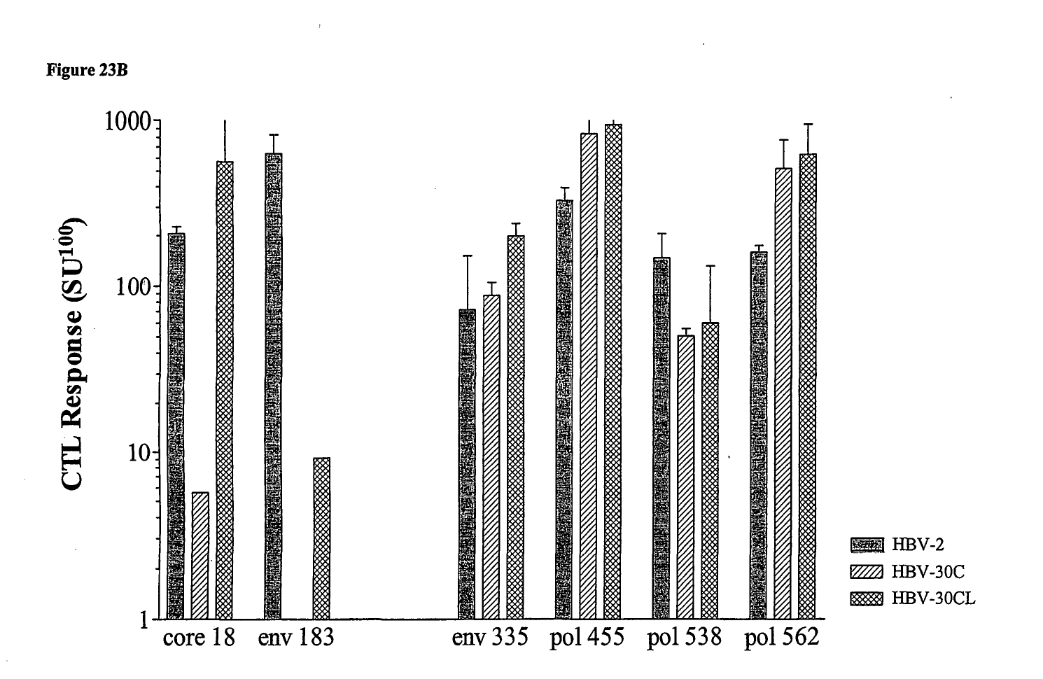

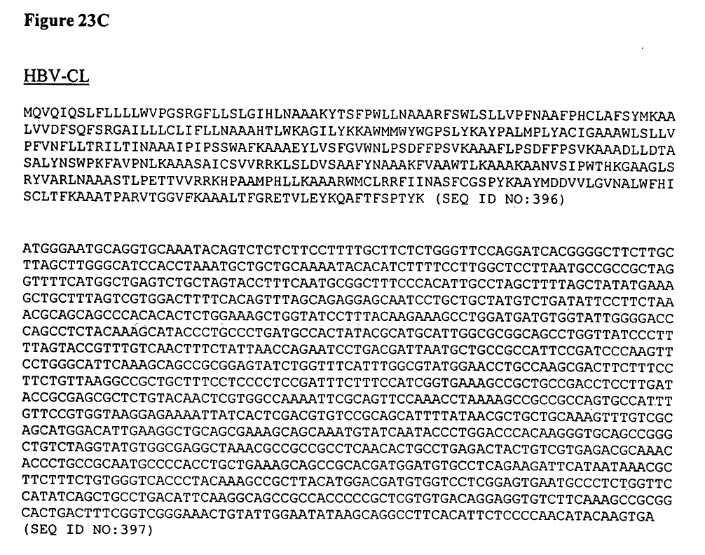

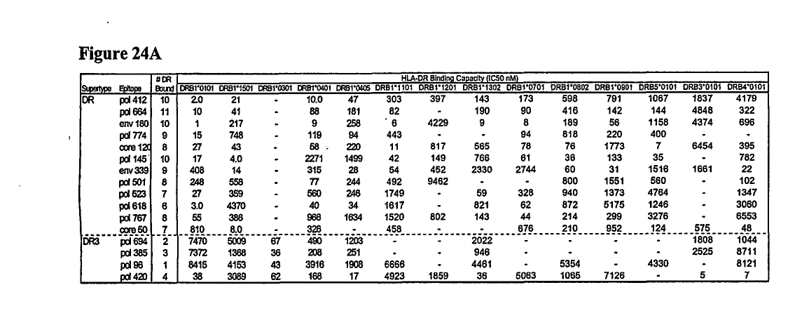

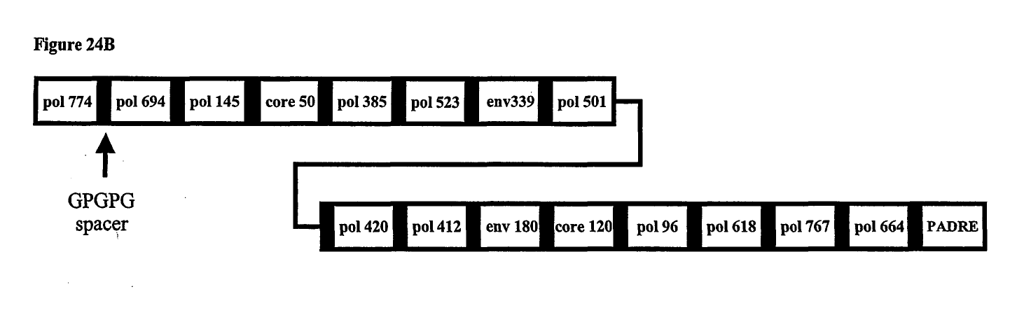

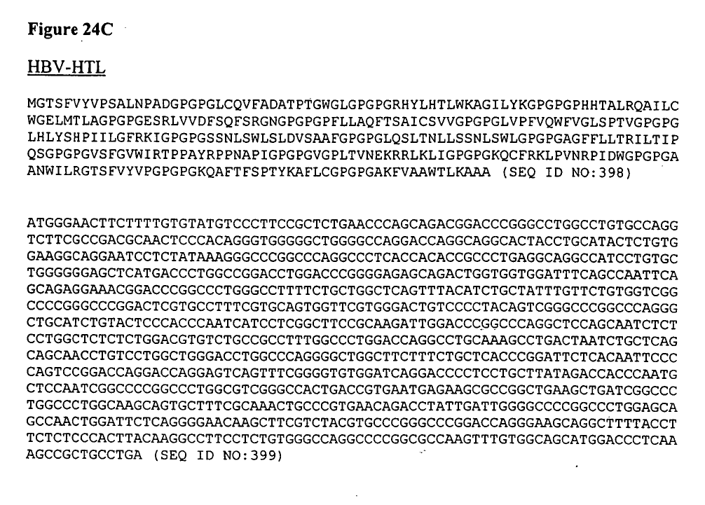

Given the methods described herein for optimizing epitope configuration and spacers between the epitopes, the skilled artisan may include any HLA epitopes into the multi-epitope constructs described herein. FIGS. 2, 3, 9, 17, and 18A-18N depict exemplary constructs, where epitopes in the constructs are listed in FIGS. 19A-19E. Exemplary constructs are also set forth in FIGS. 20B, 20D, 20E, and 20F (epitopes are listed in FIG. 20A); FIGS. 21B, 21D, and 21E (epitopes are listed in FIG. 21A); FIGS. 22B, 22D, and 22E (epitopes are listed in 22A); FIG. 23C; and FIGS. 24B and 24C (epitopes are listed in FIG. 24A). Multi-epitope constructs may include five or more, or six, seven, eight, nine, ten, eleven, twelve, thirteen, fourteen, fifteen, twenty, twenty-five, or thirty or more of the epitopes set forth in FIGS. 19A-19E, 20A, 21A, 22A, and 24A. Multi-epitope constructs that include any combinations of these epitopes can be optimized using the procedures set forth herein, and spacers can be optimized as well.

Multi-epitope constructs can be generated using methodology well known in the art. For example, polypeptides comprising the multi-epitope constructs can be synthesized and linked. Typically, multi-epitope constructs are constructed using recombinant DNA technology.

IV. Expression Vectors and Construction of a Multi-Epitope Constructs

The multi-epitope constructs of the invention are typically provided as an expression vector comprising a nucleic acid encoding the multi-epitope polypeptide. Construction of such expression vectors is described, for example in PCT/US99/10646. The expression vectors contain at least one promoter element that is capable of expressing a transcription unit encoding the nucleic acid in the appropriate cells of an organism so that the antigen is expressed and targeted to the appropriate HLA molecule. For example, for administration to a human, a promoter element that functions in a human cell is incorporated into the expression vector.

In preferred embodiments, the invention utilizes routine techniques in the field of recombinant genetics. Basic texts disclosing the general methods of use in this invention include Sambrook et al., Molecular Cloning, A Laboratory Manual (2nd ed. 1989); Kriegler, Gene Transfer and Expression: A Laboratory Manual (1990); and Current Protocols in Molecular Biology (Ausubel et al., eds., 1994); Oligonucleotide Synthesis. A Practical Approach (Gait, ed., 1984); Kuijpers, Nucleic Acids Research 18(17):5197 (1994); Dueholm, J. Org. Chem. 59:5767-5773 (1994); Methods in Molecular Biology, volume 20 (Agrawal, ed.); and Tijssen, Laboratory Techniques in Biochemistry and Molecular Biology—Hybridization with Nucleic Acid Probes, e.g., Part I, chapter 2 “Overview of principles of hybridization and the strategy of nucleic acid probe assays” (1993)).

The nucleic acids encoding the epitopes are assembled in a construct according to standard techniques. In general, the nucleic acid sequences encoding multi-epitope polypeptides are isolated using amplification techniques with oligonucleotide primers, or are chemically synthesized. Recombinant cloning techniques can also be used when appropriate. Oligonucleotide sequences are selected which either amplify (when using PCR to assemble the construct) or encode (when using synthetic oligonucleotides to assemble the construct) the desired epitopes.

Amplification techniques using primers are typically used to amplify and isolate sequences encoding the epitopes of choice from DNA or RNA (see U.S. Pat. Nos. 4,683,195 and 4,683,202; PCR Protocols: A Guide to Methods and Applications (Innis et al., eds, 1990)). Methods such as polymerase chain reaction (PCR) and ligase chain reaction (LCR) can be used to amplify epitope nucleic acid sequences directly from MRNA, from cDNA, from genomic libraries or cDNA libraries. Restriction endonuclease sites can be incorporated into the primers. Multi-epitope constructs amplified by the PCR reaction can be purified from agarose gels and cloned into an appropriate vector.

Synthetic oligonucleotides can also be used to construct multi-epitope constructs. This method is performed using a series of overlapping oligonucleotides, representing both the sense and non-sense strands of the gene. These DNA fragments are then annealed, ligated and cloned. Oligonucleotides that are not commercially available can be chemically synthesized according to the solid phase phosphoramidite triester method first described by Beaucage & Caruthers, Tetrahedron Letts. 22:1859-1862 (1981), using an automated synthesizer, as described in Van Devanter et. al., Nucleic Acids Res. 12:6159-6168 (1984). Purification of oligonucleotides is by either native acrylamide gel electrophoresis or by anion-exchange HPLC as described in Pearson & Reanier, J. Chrom. 255:137-149 (1983).

The epitopes of the multi-epitope constructs are typically subcloned into an expression vector that contains a strong promoter to direct transcription, as well as other regulatory sequences such as enhancers and polyadenylation sites. Suitable promoters are well known in the art and described, e.g., in Sambrook et al. and Ausubel et al. Eukaryotic expression systems for mammalian cells are well known in the art and are commercially available. Such promoter elements include, for example, cytomegalovirus (CMV), Rous sarcoma virus LTR and SV40.

The expression vector typically contains a transcription unit or expression cassette that contains all the additional elements required for the expression of the multi-epitope construct in host cells. A typical expression cassette thus contains a promoter operably linked to the multi-epitope construct and signals required for efficient polyadenylation of the transcript. Additional elements of the cassette may include enhancers and introns with functional splice donor and acceptor sites.

In addition to a promoter sequence, the expression cassette can also contain a transcription termination region downstream of the structural gene to provide for efficient termination. The termination region may be obtained from the same gene as the promoter sequence or may be obtained from different genes.

The particular expression vector used to transport the genetic information into the cell is not particularly critical. Any of the conventional vectors used for expression in eukaryotic cells may be used. Expression vectors containing regulatory elements from eukaryotic viruses are typically used in eukaryotic expression vectors, e.g., SV40 vectors, CMV vectors, papilloma virus vectors, and vectors derived from Epstein Bar virus.

The multi-epitope constructs of the invention can be expressed from a variety of vectors including plasmid vectors as well as viral or bacterial vectors. Examples of viral expression vectors include attenuated viral hosts, such as vaccinia or fowlpox. As an example of this approach, vaccinia virus is used as a vector to express nucleotide sequences that encode the peptides of the invention. Upon introduction into a host bearing a tumor, the recombinant vaccinia virus expresses the immunogenic peptide, and thereby elicits a host CTL and/or HTL response. Vaccinia vectors and methods useful in immunization protocols are described in, e.g., U.S. Pat. No. 4,722,848.

A wide variety of other vectors useful for therapeutic administration or immunization, e.g. adeno and adeno-associated virus vectors, retroviral vectors, non-viral vectors such as BCG (Bacille Calmette Guerin), Salmonella typhi vectors, detoxified anthrax toxin vectors, and the like, will be apparent to those skilled in the art.

Immunogenicity and antigenicity of the multi-epitope constructs are evaluated as described herein.

Targeting Sequences

The expression vectors of the invention may encode one or more MHC epitopes operably linked to a MHC targeting sequence, and are referred to herein as “targeting nucleic acids” or “targeting sequences.” The use of a MHC targeting sequence enhances the immune response to an antigen, relative to delivery of antigen alone, by directing the peptide epitope to the site of MHC molecule assembly and transport to the cell surface, thereby providing an increased number of MHC molecule-peptide epitope complexes available for binding to and activation of T cells.

MHC Class I targeting sequences can be used in the present invention, e.g., those sequences that target an MHC Class I epitope peptide to a cytosolic pathway or to the endoplasmic reticulum (see, e.g., Rammensee et al., Inmunogenetics 41:178-228 (1995)). For example, the cytosolic pathway processes endogenous antigens that are expressed inside the cell. Although not wishing to be bound by any particular theory, cytosolic proteins are thought to be at least partially degraded by an endopeptidase activity of a proteasome and then transported to the endoplasmic reticulum by the TAP molecule (transporter associated with processing). In the endoplasmic reticulum, the antigen binds to MHC Class I molecules. Endoplasmic reticulum signal sequences bypass the cytosolic processing pathway and directly target endogenous antigens to the endoplasmic reticulum, where proteolytic degradation into peptide fragments occurs. Such MHC Class I targeting sequences are well known in the art, and include, e.g., signal sequences such as those from Ig kappa, tissue plasminogen activator or insulin. A preferred signal peptide is the human. Ig kappa chain sequence. Endoplasmic reticulum signal sequences can also be used to target MHC Class II epitopes to the endoplasmic reticulum, the site of MHC Class I molecule assembly. MHC Class II targeting sequences can also be used in the invention, e.g., those that target a peptide to the endocytic pathway. These targeting sequences typically direct extracellular antigens to enter the endocytic pathway, which results in the antigen being transferred to the lysosomal compartment where the antigen is proteolytically cleaved into antigen peptides for binding to MHC Class II molecules. As with the normal processing of exogenous antigen, a sequence that directs a MHC Class II epitope to the endosomes of the endocytic pathway and/or subsequently to lysosomes, where the MHC Class II epitope can bind to a MHC Class II molecule, is a MHC Class II targeting sequence. For example, group of MHC Class II targeting sequences useful in the invention are lysosomal targeting sequences, which localize polypeptides to lysosomes. Since MHC Class II molecules typically bind to antigen peptides derived from proteolytic processing of endocytosed antigens in lysosomes, a lysosomal targeting sequence can function as a MHC Class II targeting sequence. Lysosomal targeting sequences are well known in the art and include sequences found in the lysosomal proteins LAMP-1 and LAMP-2 as described by August et al. U.S. Pat. No. 5,633,234, issued May 27, 1997), which is incorporated herein by reference.

Other lysosomal proteins that contain lysosomal targeting sequences include HLA-DM. HLA-DM is an endosomal/lysosomal protein that functions in facilitating binding of antigen peptides to MHC Class II molecules. Since it is located in the lysosome, HLA-DM has a lysosomal targeting sequence that can function as a MHC Class II molecule targeting sequence (Copier et al., J. Immunol. 157:1017-1027 (1996), which is incorporated herein by reference).

The resident lysosomal protein HLA-DO can also function as a lysosomal targeting sequence. In contrast to the above described resident lysosomal proteins LAMP-1 and HLA-DM, which encode specific Tyr-containing motifs that target proteins to lysosomes, HLA-DO is targeted to lysosomes by association with HLA-DM (Liljedahl et al., EMBO J. 15:4817-4824 (1996)), which is incorporated herein by reference. Therefore, the sequences of HLA-DO that cause association with HLA-DM and, consequently, translocation of HLA-DO to lysosomes can be used as MHC Class II targeting sequences. Similarly, the murine homolog of HLA-DO, H2-DO, can be used to derive a MHC Class II targeting sequence. A MHC Class II epitope can be fused to HLA-DO or H2-DO and targeted to lysosomes.

In another example, the cytoplasmic domains of B cell receptor subunits Ig-α and Ig-β mediate antigen internalization and increase the efficiency of antigen presentation as described in, for example, Bonnerot et al., Immunity 3:335-347 (1995). Therefore, the cytoplasmic domains of the Ig-α and Ig-β proteins can function as MHC Class II targeting sequences that target a MHC Class II epitope to the endocytic pathway for processing and binding to MHC Class II molecules.

Another example of a MHC Class II targeting sequence that directs MHC Class II epitopes to the endocytic pathway is a sequence that directs polypeptides to be secreted, where the polypeptide can enter the endosomal pathway. These MHC Class II targeting sequences that direct polypeptides to be secreted mimic the normal pathway by which exogenous, extracellular antigens are processed into peptides that bind to MHC Class II molecules. Any signal sequence that functions to direct a polypeptide through the endoplasmic reticulum and ultimately to be secreted can function as a MHC Class II targeting sequence so long as the secreted polypeptide can enter the endosomal/lysosomal pathway and be cleaved into peptides that can bind to MHC Class II molecules.

In another example, the Ii protein binds to MHC Class IMHC Class II molecules in the endoplasmic reticulum, where it functions to prevent peptides present in the endoplasmic reticulum from binding to the MHC Class II molecules. Therefore, fusion of a MHC Class II epitope to the Ii protein targets the MHC Class II epitope to the endoplasmic reticulum and a MHC Class II molecule. For example, the CLIP sequence of the Ii protein can be removed and replaced with a MHC Class IMHC Class II epitope sequence so that the MHC Class II epitope is directed to the endoplasmic reticulum, where the epitope binds to a MHC Class II molecule.

In some cases, antigens themselves can serve as MHC Class II or I targeting sequences and can be fused to a universal MHC Class II epitope to stimulate an immune response. Although cytoplasmic viral antigens are generally processed and presented as complexes with MHC Class I molecules, long-lived cytoplasmic proteins such as the influenza matrix protein can enter the MHC Class IMHC Class II molecule processing pathway as described in, for example, Gueguen & Long, Proc. Natl. Acad. Sci. USA 93:14692-14697 (1996). Therefore, long-lived cytoplasmic proteins can function as a MHC Class IMHC Class II targeting sequence. For example, an expression vector encoding influenza matrix protein fused to a universal MHC Class IMHC Class II epitope can be advantageously used to target influenza antigen and the universal MHC Class IMHC Class II epitope to the MHC Class IMHC Class II pathway for stimulating an immune response to influenza.

Other examples of antigens functioning as MHC Class IMHC Class II targeting sequences include polypeptides that spontaneously form particles. The polypeptides are secreted from the cell that produces them and spontaneously form particles, which are taken up into an antigen-presenting cell by endocytosis such as receptor-mediated endocytosis or are engulfed by phagocytosis. The particles are proteolytically cleaved into antigen peptides after entering the endosomal/lysosomal pathway.

One such polypeptide that spontaneously forms particles is HBV surface antigen (HBV-S) as described in, for example, Diminsky et al., Vaccine 15:637-647 (1997) or Le Borgne et al., Virology 240:304-315 (1998). Another polypeptide that spontaneously forms particles is HBV core antigen as described in, for example, Kuhröber et al., International Immunol. 9:1203-1212 (1997). Still another polypeptide that spontaneously forms particles is the yeast Ty protein as described in, for example, Weber et al., Vaccine 13:831-834 (1995). For example, an expression vector containing HBV-S antigen fused to a universal MHC Class IMHC Class II epitope can be advantageously used to target HBV-S antigen and the universal MHC Class IMHC Class II epitope to the MHC Class IMHC Class II pathway for stimulating an immune response to HBV.

Administration In Vivo

The invention also provides methods for stimulating an immune response by administering an expression vector of the invention to an individual. Administration of an expression vector of the invention for stimulating an immune response is advantageous because the expression vectors of the invention target MHC epitopes to MHC molecules, thus increasing the number of CTL and HTL activated by the antigens encoded by the expression vector.

Initially, the expression vectors of the invention are screened in mouse to determine the expression vectors having optimal activity in stimulating a desired immune response. Initial studies are therefore carried out, where possible, with mouse genes of the MHC targeting sequences. Methods of determining the activity of the expression vectors of the invention are well known in the art and include, for example, the uptake of ³H-thymidine to measure T cell activation and the release of ⁵¹Cr to measure CTL activity as described below in Examples II and III. Experiments similar to those described in Example IV are performed to determine the expression vectors having activity at stimulating an immune response. The expression vectors having activity are further tested in human. To circumvent potential adverse immunological responses to encoded mouse sequences, the expression vectors having activity are modified so that the MHC Class IMHC Class II targeting sequences are derived from human genes. For example, substitution of the analogous regions of the human homologs of genes containing various MHC Class IMHC Class II targeting sequences are substituted into the expression vectors of the invention. Expression vectors containing human MHC Class IMHC Class II targeting sequences, such as those described in Example I below, are tested for activity at stimulating an immune response in human.

The invention also relates to pharmaceutical compositions comprising a pharmaceutically acceptable carrier and an expression vector of the invention. Pharmaceutically acceptable carriers are well known in the art and include aqueous or non-aqueous solutions, suspensions and emulsions, including physiologically buffered saline, alcohol/aqueous solutions or other solvents or vehicles such as glycols, glycerol, oils such as olive oil or injectable organic esters.

A pharmaceutically acceptable carrier can contain physiologically acceptable compounds that act, for example, to stabilize the expression vector or increase the absorption of the expression vector. Such physiologically acceptable compounds include, for example, carbohydrates, such as glucose, sucrose or dextrans, antioxidants such as ascorbic acid or glutathione, chelating agents, low molecular weight polypeptides, antimicrobial agents, inert gases or other stabilizers or excipients. Expression vectors can additionally be complexed with other components such as peptides, polypeptides and carbohydrates. Expression vectors can also be complexed to particles or beads that can be administered to an individual, for example, using a vaccine gun. One skilled in the art would know that the choice of a pharmaceutically acceptable carrier, including a physiologically acceptable compound, depends, for example, on the route of administration of the expression vector.

The invention further relates to methods of administering a pharmaceutical composition comprising an expression vector of the invention to stimulate an immune response. The expression vectors are administered by methods well known in the art as described in, for example, Donnelly et al. (Ann. Rev. Immunol. 15:617-648 (1997)); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997). In one embodiment, the multi-epitope construct is administered as naked nucleic acid.

A pharmaceutical composition comprising an expression vector of the invention can be administered to stimulate an immune response in a subject by various routes including, for example, orally, intravaginally, rectally, or parenterally, such as intravenously, intramuscularly, subcutaneously, intraorbitally, intracapsularly, intraperitoneally, intracisternally or by passive or facilitated absorption through the skin using, for example, a skin patch or transdermal iontophoresis, respectively. Furthermore, the composition can be administered by injection, intubation or topically, the latter of which can be passive, for example, by direct application of an ointment or powder, or active, for example, using a nasal spray or inhalant. An expression vector also can be administered as a topical spray, in which case one component of the composition is an appropriate propellant. The pharmaceutical composition also can be incorporated, if desired, into liposomes, microspheres or other polymer matrices as described in, for example, Felgner et al., U.S. Pat. No. 5,703,055; Gregoriadis, Liposome Technology, Vols. I to III (2nd ed. 1993). Liposomes, for example, which consist of phospholipids or other lipids, are nontoxic, physiologically acceptable and metabolizable carriers that are relatively simple to make and administer.

The expression vectors of the invention can be delivered to the interstitial spaces of tissues of an animal body as described in, for example, Felgner et al., U.S. Pat. Nos. 5,580,859 and 5,703,055. Administration of expression vectors of the invention to muscle is a particularly effective method of administration, including intradermal and subcutaneous injections and transdermal administration. Transdermal administration, such as by iontophoresis, is also an effective method to deliver expression vectors of the invention to muscle. Epidermal administration of expression vectors of the invention can also be employed. Epidermal administration involves mechanically or chemically irritating the outermost layer of epidermis to stimulate an immune response to the irritant (Carson et al., U.S. Pat. No. 5,679,647).

Other effective methods of administering an expression vector of the invention to stimulate an immune response include mucosal administration as described in, for example, Carson et al., U.S. Pat. No. 5,679,647. For mucosal administration, the most effective method of administration includes intranasal administration of an appropriate aerosol containing the expression vector and a pharmaceutical composition. Suppositories and topical preparations are also effective for delivery of expression vectors to mucosal tissues of genital, vaginal and ocular sites. Additionally, expression vectors can be complexed to particles and administered by a vaccine gun.

The dosage to be administered is dependent on the method of administration and will generally be between about 0.1 μg up to about 200 μg. For example, the dosage can be from about 0.05 μg/kg to about 50 mg/kg, in particular about 0.005-5 mg/kg. An effective dose can be determined, for example, by measuring the immune response after administration of an expression vector. For example, the production of antibodies specific for the MHC Class II epitopes or MHC Class I epitopes encoded by the expression vector can be measured by methods well known in the art, including ELISA or other immunological assays. In addition, the activation of T helper cells or a CTL response can be measured by methods well known in the art including, for example, the uptake of ³H-thymidine to measure T cell activation and the release of ⁵¹Cr to measure CTL activity (see Examples II and III below).

The pharmaceutical compositions comprising an expression vector of the invention can be administered to mammals, particularly humans, for prophylactic or therapeutic purposes. Examples of diseases that can be treated or prevented using the expression vectors of the invention include infection with HBV, HCV, HIV and CMV as well as prostate cancer, renal carcinoma, cervical carcinoma, lymphoma, condyloma acuminatum and acquired immunodeficiency syndrome (AIDS).

In therapeutic applications, the expression vectors of the invention are administered to an individual already suffering from cancer, autoimmune disease or infected with a virus. Those in the incubation phase or acute phase of the disease can be treated with expression vectors of the invention, including those expressing all universal MHC Class II epitopes, separately or in conjunction with other treatments, as appropriate.

In therapeutic and prophylactic applications, pharmaceutical compositions comprising expression vectors of the invention are administered to a patient in an amount sufficient to elicit an effective immune response to an antigen and to ameliorate the signs or symptoms of a disease. The amount of expression vector to administer that is sufficient to ameliorate the signs or symptoms of a disease is termed a therapeutically effective dose. The amount of expression vector sufficient to achieve a therapeutically effective dose will depend on the pharmaceutical composition comprising an expression vector of the invention, the manner of administration, the state and severity of the disease being treated, the weight and general state of health of the patient and the judgment of the prescribing physician.

### EXAMPLES

The following examples are offered to illustrate, but not to limit the claimed invention. It is understood that the examples and embodiments described herein are for illustrative purposes only and that various modifications or changes in light thereof are suggested to persons skilled in the art and are to be included within the spirit and purview of this application and scope of the appended claims.

Examples 1-9 provide examples of assays for evaluating the immunogenicity and antigenicity of multi-epitope constructs.

### Example 1

Antigenicity Assays

High-affinity peptide-specific CTL lines can be generated from splenocytes of transgenic mice that have been primed with DNA, peptide/IFA, or lipopeptide. Briefly, splenocytes from transgenic mice are stimulated 0.1 μg/ml peptide and LPS blasts. Ten days after the initial stimulation, and weekly thereafter, cells are restimulated with LPS blasts pulsed for 1 hour with 0.1 μg/ml peptide. CTL lines are assayed 5 days following restimulation in an in situ IFNγ ELISA as described above. Alternatively, CTL lines that are derived from, e.g., patients infected with the targeted pathogen or who have the targeted disease, e.g., cancer, can be used. Specific CTL lines that are not available either from transgenic mice or from patients are generated from PBMC of normal donors, drawing on the expertise in the art.

Target cells to be used in these assays are Jurkat or 0.221 cells transfected with A2.1/K^{b}, A11/K^{b}, A1/K^{b}, or B7/K^{b} for CTL lines derived from transgenic mice. All these cell lines are currently available to us (Epimmune Inc., San Diego, Calif.). In the case of human CTL lines, 0.221 cells transfected with the appropriate human HLA allele are utilized. We currently have 0.221 cells transfected with A2 and A1, and are generating A11, A24 and B7 transfectants. In an alternative embodiment, if unforeseen problems arise in respect to target cells, LPS blasts and EBV-transformed lines are utilized for murine and human CTL lines, respectively.

To assay for antigenicity, serially diluted CTLs are incubated with 105 target cells and multiple peptide concentrations ranging from 1 to 10⁻⁶ μg/ml. In addition, CTLs are also incubated with target cells transfected with an episomal vector containing a multi-epitope construct of interest. Episomal vectors are known in the art.

The relative amount of peptide generated by natural processing within the multi-epitope nucleic acid-transfected APCs is quantitated as follows. The amount of IFNγ generated by the CTL lines upon recognition of the transfected target cells are recorded. The amount of synthetic peptide necessary to yield the same amount of IFNγ are interpolated from a standard curve generated when the same CTL line is incubated in parallel with known concentrations of peptide.

### Example 2

Mice, Immunizations and Cell Cultures

The derivation of the HLA-A2.1/K^{b} (Vitiello et al., J Exp Med, Vol. 173(4):1007-15 (1991)) and A11K^{b} (Alexander et al., J Immunol, Vol. 159(10):4753-61 (1997)) transgenic mice used in this study has been described. HLA B7 K^{b} transgenic mice are available in house (Epimmune Inc., San Diego, Calif.). HLA DR2, DR3 and DR4 transgenic mice are obtained from C. David (Mayo Clinic). Non-transgenic H-2^{b} mice are purchased from Charles River Laboratories or other commercial vendors. Immunizations are performed as described in (Ishioka et al., J Immunol, Vol. 162(i):3915-25 (1999)). All cells are grown in culture medium consisting of RPMI 1640 medium with HEPES (Gibco Life Technologies) supplemented with 10% FBS, 4 mM L-glutamine, 50 μM 2-ME, 0.5 mM sodium pyruvate, 100 μg/ml streptomycin and 100 U/ml penicillin.

HLA transgenic mice and antigenicity assays are used for the purpose of testing and optimization CTL responses. The natural crossreactivity between HLA-DR and IA^{b} can also be exploited to test HTL responses. This evaluation provides an assessment of the antigenicity and immunogenicity of multi-epitope constructs.

### Example 3

Proliferation Assays

To assess the ability of HTL epitopes to induce an immune response, assays such as proliferation assays are often performed. For example, mouse CD4 T lymphocytes are immunomagnetically isolated from splenic single cell suspensions using DynaBeads Mouse CD4 (L3T4) (Dynal). Briefly, 2×10⁷ spleen cells are incubated with 5.6×10⁷ magnetic beads for 40 minutes at 4° C., and then washed 3 times. Magnetic beads are detached using DetachaBead Mouse CD4 (Dynal). Isolated CD4 T lymphocytes (2×10⁵ cells/well) are cultured with 5×10⁵ irradiated (3500 rad) syngeneic spleen cells in triplicate in flat-bottom 96-well microtiter plates. Purified peptides are added to wells at a final concentration of 20, 1, 0.05 and 0 μg/ml and cells are cultured for a total of 4 days. Approximately 14 hour before harvesting, 1 μCi of ³H-thymidine (ICN) is added to each well. The wells are harvested onto Unifilter GF/B plates (Packard) using the Filtermate Harvester Packard). ³H-Thymidine incorporation is determined by liquid scintillation counting using the TopCount™ microplate scintillation counter (Packard).

### Example 4

⁵¹Chromium Release Assay

This assay to measure CTL activity is well known in the art. The assay quantifies the lytic activity of the T cell population by measuring the percent ⁵¹Cr released from a ⁵¹Cr-labeled target population (Brunner et al., Immunology, Vol. 14(2):181-96 (1968)). Data derived from the chromium release assay is usually expressed either as a CTL frequency/10⁶ cell (limiting dilution analysis, LDA; (Current Protocols in Immunology, Vol 1, John Wiley & Sons, Inc., USA 1991 Chapter 3; Manual of Clinical Laboratory Immunology, Fifth edition, ASM Press, 1997 Section R), or by a less cumbersome quantitative assessment of bulk CTL activity (lytic Units; LU assay). In a LU assay, the standard E:T ratio versus percent cytotoxicity data curves generated in a ⁵¹Cr-release assay are converted into lytic units (LU) per 10⁶ effector cells, with 1 LU defined as the lytic activity required to achieve 30% lysis of target cells (Wunderlick, J., Shearer, G., and Livingston, A. In: J. Coligan, A. Kruisbeek, D. Margulies, E. Shevach, and W. Strober (Eds.), Current Protocols in Immunology, Vol 1, “Assays for T cell function: induction and measurement of cytotoxic T lymphocyte activity.” John Wiley & Sons, Inc., USA, p. 3.11.18). The LU calculation allows quantifying responses and thus readily comparing different experimental values.

### Example 5

In Situ IFNγ ELISA

An in situ IFNγ ELISA assay has been developed and optimized for both freshly isolated and peptide-restimulated splenocytes (see, e.g., McKinney et al., J. Immunol. Meth. 237 (1-2):105-117 (2000))IFN. This assay is based on the ELISPOT assay, but utilizes a soluble chromagen, making it readily adaptable to high-throughput analysis. In both the primary and restimulation assays, this technique is more sensitive than either a traditional supernatant ELISA or the ⁵¹Cr-release assay, in that responses are observed in the in situ ELISA that are not detectable in these other assays. On a per cell basis, the sensitivity of the in situ ELISA is approximately one IFNγ secreting cell/10⁴ plated cells.

96-well ELISA plates are coated with anti-IFNγ (rat anti-mouse IFNα MAb, Clone R4-6A2, Pharmingen) overnight at 4° C., and then blocked for 2 hours at room temperature with 10% FBS in PBS. Serially diluted primary splenocytes or CTLs are cultured for 20 hours with peptide and 10⁵ Jurkat A2.1/K^{b} cells/well at 37° C. with 5% CO₂. The following day, the cells are washed out and the amount of IFNγ that had been secreted into the wells is detected in a sandwich ELISA, using biotinylated α-IFNγ (rat anti-mouse IFNγ mAb, Clone XMG1.2, Pharmingen) to detect the secreted IFNγ. HRP-coupled strepavidin (Zymed) and TMB (ImmunoPure® TMB Substrate Kit, Pierce) are used according to the manufacturer's directions for color development. The absorbance is read at 450 nm on a Labsystems Multiskan RC ELISA plate reader. In situ IFNγ ELISA data is evaluated in secretory units (SU), based on the number of cells that secrete 100 pg of IFNγ in response to a particular peptide, corrected for the background amount of IFN in the absence of peptide.

### Example 6

ELISPOT Assay

The ELISPOT assay quantifies the frequency of T cells specific for a given peptide by measuring the capacity of individual cells to be induced to produce and release specific lymphokines, usually IFNγ. The increased sensitivity of the ELISPOT assay has allowed investigators to detect responses from cells freshly isolated from infected humans or experimental animals (Murali-Krishna et al., Immunity, Vol. 8(2):177-87 (1998); Ogg et al., Science, Vol. 279(5359):2103-6 (1998)). The ELISPOT assays are conducted as described above for the IFNγ ELISA until the final steps, where ExtrAvidin-AP (Sigma, 1:500. dilution) is used in place HRP-strepavidin. Color is developed using the substrate 5-BCIP (BioRad) according to the manufacturer's directions. Spots are counted using a phase contrast microscope. Alternatively, spots are counted utilizing the Zeiss KS ELISPOT reader. In this case the BCIP/NBT substrate is used.

The ELISPOT assay is routinely utilized to quantitate immune responses. The spots can be manually counted, however, in a preferred mode, a KS ELISPOT reader from Zeiss, a microscope-based system with software specifically designed to recognize and count spots is used.

### Example 7

Tetramer Staining

Tetramer staining is a flow cytometric technique that detects epitope-specific human CD8⁺ T-lymphocytes based on the interaction between the peptide epitope, class I antigen and the T-cell receptor specific for the epitope. This assay allows for the rapid quantitation of epitope specific human CD8⁺ T-lymphocytes in freshly isolated blood samples. MHC tetramers for various HIV peptide/HLA combinations, obtained, e.g., from the NIH repository (Tetramer Core Facility: http://www.miaid.nih.gov/reposit/tetramer/index.html). To label epitope-specific cells, 1×10⁶ PBMC in a 100 μl volume are incubated in the dark for 40 minutes with 5 μg/ml of the appropriate tetramer plus monoclonal antibodies that recognize human CD3 and CD8 (available in different fluorochrome-conjugated forms from commercial sources including PharMingen, San Diego, Calif. or BioSource, Carnarillo, Calif.). The cells are washed and paraformaldehyde fixed prior to analysis using a FACsan or FACSCalibur flow cytometer (Becton Dickinson Immunocytometry Systems, San Jose, Calif.). Sample data are analyzed using CellQuest software.

### Example 8

Assays from Clinical Samples

Various assays to evaluate the specific CD8⁺ CTL activity in frozen PBMC samples from patients or volunteers can be used. ELISPOT, chromium release, in situ IFNγ release, proliferation and tetramer assays are all useful to evaluate responses from various experimental models, e.g., those of murine and/or primate origin.

Experimental methods for the murine version of these assays are described above, and these are adapted to human systems as described (Livingston et al, J Immunol, Vol. 159(3):1383-92 (1997); Heathcote et al., Hepatology, Vol. 30(2):531-6 (1999); Livingston et al., J Immunol, Vol. 162(5):3088-95 (1999)) and can be further adapted a recognized by one of ordinary skill in the art. Calculations on the amounts of frozen PBMC samples necessary to complete the assays are also described greater detail in Example 14.

### Example 9

Transgenic Animals

Transgenic mice (HLA-A2.1/K^{b}H2^{b}; HLA-A11/K^{b}; HLA-B7/K^{b}) are immunized intramuscularly in the anterior tibialis muscle or subcutaneously in the base of the tail with doses up to 100 μg of DNA or peptide in 10-100 μl volumes. DNA is formulated in saline, and peptides in IFA. 11-21 days later, the animals are sacrificed using CO₂ asphyxiation, their spleens removed and used as the source of cells for in vitro determination of CTL function. Typically, 3-6 mice per experimental group are used. In addition, spleens from non-immunized mice are used as a source of APC for restimulation of CTL cultures. Both males and females of 8-12 weeks of age are used.

### Example 10

Demonstration of Simultaneous Induction of Responses Against Multiple CTL and HTL Epitopes

Construction and testing of CTL epitope strings:

This example provides an example of testing mutliple CTL and HTL epitopes. For example, epitope strings encompassing 10-12 different CTL epitopes under the control of a single promoter are synthesized and incorporated in a standard plasmid, pcDNA 3.1 (Invitrogen, San Diego). These constructs include a standard signal sequence and a universal HTL epitope, PADRE®. Each set of epitopes is chosen to allow balanced population coverage. To facilitate testing and optimization, a balanced representation of epitopes that have been shown to be immunogenic in transgenic mice, and/or antigenic in humans are included.

The specific order of these CTL epitopes is chosen to minimize Class I junctional motifs by the use of the computer program, as described herein. If, despite best efforts regarding order optimization, potential junctional epitopes are still present in a construct in accordance with the invention, corresponding peptides are synthesized to monitor for CTL responses against such epitopes in HLA transgenic mice. Generally, minimization of junctional motifs is successful and adequate. However, if responses against any junctional epitopes are detected, these junctional epitopes are disrupted by the use of short one to two residue spacers, such as K, AK, KA, KK, or A, compatible with expected proteolytic cleavage preferences discussed in the previous sections.

Since the ultimate use of optimized constructs is a human vaccine, optimized human codons are utilized. However, to facilitate the optimization process in HLA transgenic mice, care are applied to select, whenever possible, human codons that are also optimal for mice. Human and murine codon usage is very similar. See, for example, Codon usage database at http://www.kazusa.or.jp/codon/.

Human cells transfected with the various multi-epitope nucleic acid vaccine constructs can be used in antigenicity assays, conducted in parallel with in vivo testing in HLA transgenic mice. Any potential discrepancy between multi-epitope nucleic acid vaccine efficacy, due to the differential codon usage, is addressed by the availability of these two different assay systems.

Typically, antigenicity and immunogenicity testing of plasmid constructs is conducted in parallel. In vivo testing in transgenic mice are performed for A2, A11, and B7 HLA transgenic mice. Following a protocol well established in our laboratory, cardiotoxin pretreated mice are injected i.m. with 100 μg of each plasmid and responses evaluated eleven days later (Ishioka et al., J Immunol, Vol. 162(7):3915-25 (1999)). Assays will include ELISPOT from freshly isolated cells, as well as interferon gamma release and cytotoxicity chromium release assays from restimulated cell cultures. All of the above mentioned techniques are well established in the art. The simultaneous measurement of responses against epitopes is not problematic, as large colonies of transgenic mice are already established “in house” for these HLA types. Groups of four to six mice are adequate to measure responses against six to ten different epitopes, in multiple readout assays. Testing of HLA A2-restricted, HIV-derived epitopes in HLA A2 transgenic mice is typically employed. However, should problems be encountered, antigenicity testing using human APC can be used as an alternative strategy, or, can be used to complement the transgenic mice studies.

For the purpose of extending the correlation between immunogenicity in transgenic animals and antigenicity, as noted in the studies reported herein, antigenicity testing is utilized to evaluate responses against epitopes such as Pol 498, Env 134, Nef 221, Gag 271, for which high affinity CTL lines are already available in house. For the purpose of generating additional suitable CTL lines, direct immunization of HLA transgenic mice with peptides emulsified in adjuvant, or lipopeptides are utilized, as described herein, and routinely applied in our laboratory, to generate lines for use in antigenicity assays.

Antigenicity assays are also used, as a primary readout for epitopes for which in vivo optimization experiments are not feasible. These epitopes include A24 and possibly A1 restricted epitopes, as well as any epitope which is non-immunogenic in HLA transgenic mice. In any such cases, we use human CTL lines, generated from pathogen exposed individuals. Alternatively, we generate CTL lines for in vitro CTL induction, using GMCSF/IL4-induced dendritic cells and peripheral blood lymphocytes (Celis et al., Proc Natl Acad Sci USA, Vol. 91(6):2105-9 (1994)).

Episomal vectors encoding the multi-epitope constructs are generated and transfected into appropriate human cell lines to generate target cells. For example, the human T cell line Jurkat can be used, but lymphoblastoid cell lines have also been successfully utilized. For experiments utilizing CTL lines of human origin, well-characterized HLA-matched, homozygous, EBV cell lines are commonly used as a source of purified-MHC and as target cells and are used as recipients of the multi-epitope nucleic acid transfections. For experiments utilizing CTL lines derived from HLA transgenic mice, a collection of Class I negative, EBV-transformed, mutant cell lines 0.221 (Shimizu Y, DeMars R, J Immunol, Vol. 142(9):3320-8 (1989)) transfected with matching HLA/K^{b} chimeric constructs are used as the recipient of the multi-epitope nucleic acid transfections. Such cells effectively present peptide antigens to CTL lines (Celis et al., Proc Natl Acad Sci USA, Vol. 91(6):2105-9 (1994)).

Construction and Testing of HTL Epitope Strings:

Epitope strings encompassing 3-20 different HTL epitopes under the control of a single promoter are synthesized and incorporated into a standard plasmid, pcDNA 3.1 (Invitrogen, San Diego). To facilitate testing and optimization, each set of epitopes for a given construct is chosen to provide a balanced representation of epitopes which are already known to be immunogenic in IA^{b} mice. In addition, all the peptides corresponding to junctions are synthesized and tested for binding to IA^{b} and, most importantly, for binding to a panel of fourteen different DR molecules, representative of the most common DR alleles worldwide (Southwood et al., J Immunol, Vol. 160(7):3363-73 (1998)). Thus, HTL epitopes that are not directed to an antigen of interest are not created within these plasmids. However, should junctional regions with good DR binding potential (and hence, potential DR restricted immunogenicity in vivo) be detected, a spacer such as GPGPG is introduced to eliminate them. In all constructs, the number of Class I junctional motifs will also be minimized, as described herein.

Experimental vaccine plasmids are tested for immunogenicity using HLA DR transgenic mice and/or mice of the H2b haplotype. Proliferation and/or cytoline production are measured (IL5, IFNγ). In a typical protocol, cardiotoxin treated mice are injected i.m. with 100 μg of each plasmid and responses evaluated eleven days later (Ishioka et al., J Immunol, Vol. 162(7):3915-25 (1999)).

Testing for Interactions Between CTL and HTL Epitopes

The activities described above yield small, functional blocks of epitopes, which are utilized to demonstrate simultaneous responses/antigenicity against all epitopes analyzable. These blocks are the subject to further optimization, as described in the next example. Using these same constructs, immunodominance is assessed. Specifically, all the CTL epitope constructs are mixed together, or all the HTL epitope constructs are mixed together. The results obtained with the pool of constructs are then compared with the results obtained with the same construct, injected separately.

These constructs are also used to determine the effects of HTL epitopes on responses to CTL epitopes. Specifically, HTL and CTL containing plasmids are pooled and injected in mice, and CTL and HTL responses to selected epitopes are measured as described herein. Often, it is determined whether the presence, e.g., of HTL epitopes derived from the target antigen enhances CTL responses beyond the level of response attained using a plasmid-containing a pan DR binding epitope, e.g. PADRE® or a PADRE® family molecule, in the CTL epitope constructs. Typically, it is also determined whether PADRE® inhibits or augments responses to target antigen-derived HTL epitopes or conversely, if HTL epitopes derived from the antigen of interest inhibit or augment responses to PADRE®.

Responses to a large number of epitopes is attainable using this methodology. It is possible that the pooling of constructs may inhibit responses against some of the weaker epitopes. In this case, the pooling experiments are repeated after optimization.

### Example 11

Optimization of CTL and HTL Multi-Epitope Constructs

This example describes the optimization the CTL and HTL constructs described in Example 10. The potential influence of flanking residues on antigenicity and immunogenicity is also assessed in optimizing minigen constructs. These studies involve the inclusion of flanking residues, a synonym for which is “spacers,” which have been designed to facilitate effective processing.

Such an analysis can be performed as follows. First, the results of testing of the CTL multi-epitope constructs described in Example 10 are analyzed for trends and correlations between activity and the presence of specific residues at the 3 residues flanking the epitope's N- and C-termini. Epitopes for which suboptimal CTL priming is noted, that are suboptimal with respect to magnitude of response, are the targets for flanking region optimization. For each of the CTL multi-epitope nucleic acid vaccines, encoding 10-12 different CTL epitopes, ‘second generation’ multi-epitope nucleic acid vaccines, with optimized configuration, are produced.

In one embodiment, the first optimization design is to introduce either an Alanine (A) or Lysine (K) residue at position C+1 for all epitopes associated with suboptimal performance. A second optimization design is to introduce in the C+1 position, the residue naturally occurring in the target antigen, e.g., HIV, that are associated with antigenicity.

For selected epitopes, additional modifications are also introduced. Specifically, the effect of introducing other residue spacers at the epitope C- and N- termini are also investigated. Depending on the results of the analysis of the multi-epitope nucleic acid vaccines described in Example 10, residues investigated may further include, for example, G, Q, W, S and T. If junctional epitopes are created by these modifications, alternative epitope orders eliminating the junctional epitopes, are rationally designed, as described herein. All second generation constructs are tested for antigenicity and immunogenicity, as described herein.

As a result of these modifications, variations in activity that correspond to specific modifications of the multi-epitope constructs are identified. Certain modifications are found that have general, beneficial effects. To confirm this, generation and testing of additional multi-epitope nucleic acid vaccines in which all epitopes (also the ones which displayed acceptable antigenicity and immunogenicity) are subject to the same modification are conducted. In some instances, increased activity is noted for some epitopes but not others, or less desirably that certain modifications increase the activity of some, but decrease the activity of other epitopes. In such cases, additional multi-epitope nucleic acid vaccines are designed and tested, to retain the beneficial modifications, while excluding those alterations that proved to be detrimental or have no effect.

These multi-epitope nucleic acid vaccines are designated so that: a) a minimum of predicted junctional epitopes are present; and b) the epitopes which were not functional in the previous multi-epitope nucleic acid vaccines are now in a more efficacious context.

For HTL multi-epitope constructs, the data obtained from the “first generation” constructs are inspected for trends, in terms of junctional epitopes, and epitope position within the constructs, and proximity to spacers, e.g. GPGPG spacers. If specific trends are detected, second generation constructs are designed based on these trends. Alternatively, in case of multi-epitope consructs yielding suboptimal activity, the potential effectiveness of other targeting strategies, such as the ones based on Ii and LAMP are reevaluated, and compared to no targeting and simple, leader sequence targeting.

When large variations in activity of either the CTL or HTL multi-epitope constructs described in this section are detected, the results are consistent with influences such as conformational or “long-range” effects impacting construct activity. These variables can be analyzed by means of current molecular and cellular biology techniques. For example, cell lines transfected with the various multi-epitope constructs could be analyzed for mRNA expression levels, and stability by Northern analysis or primer extension assays. (Current Protocols in Molecular Biology, Vol 3, John Wiley & Sons, Inc. USA 1999).

In all multi-epitope nucleic acid vaccines, an antibody tag such as MYC/his can also be included. This tag allows for testing of protein expression levels. The inclusion of MYC/his tag (Manstein et al., Gene, Vol. 162(1):129-34 (1995)) also allows determination of the stability of the expressed products, by pulse-chase experiments. The results of these assays can then be compared with the results of the antigenicity and immunogenicity experiments. The results are inspected for the presence of trends and general rules, and correlation between the different variables examined.

### Example 12

Determination of the Simplest Plasmid Configuration Capable of Effective Delivery of Selected Epitopes

The experiments described in Examples 11 and 12 are designed to address variables concerning multi-epitope nucleic acid vaccine design. Ideally, a vector that can be used in humans is used through the entire program, but one DNA vaccine plasmid for the vaccine epitope optimization studies can be used and then switched to a vector suitable for human use. Actual vector selection is dependent on several variables. For example, the availability of vectors, suitable for human use, through a reliable source, such as the National Gene Vector Laboratory (University of Michigan) is a factor.

In this example, the optimized constructs are also ligated to form larger blocks of epitopes. All constructs are preferably designed to incorporate PADRE® peptides and leader sequence targeting in the case of CTL multi-epitope constructs. Specifically, two pairs of the 10-12 CTL epitope constructs are ligated to generate two 20-24 CTL epitope constructs. In a situation where ligation of epitopes yields suboptimal (decreased) activity compared to the smaller constructs, alternative combinations and orders of ligation are investigated. The specific pair of 20-24 CTL epitope constructs yielding optimal activity are then ligated and the resulting construct encompassing all CTL epitopes evaluated for activity. Once again up to two alternative orientations are investigated. Because of the relatively large size of this construct, the specific effect of targeting sequences are confirmed, since it is possible that leader sequence targeting are more effective on small size constructs, while larger size constructs may be most effectively targeted by ubiquitin signals. Specifically, one construct without any specific targeting sequences is generated and compared to a construct that is targeted for degradation by the addition of a ubiquitin molecule.

A similar strategy is used for HTL. Two pairs of the 3-5 HTL epitope constructs are ligated to generate two 7-9 HTL epitope constructs. Once again, in a situation where ligation of these epitopes yields suboptimal (decreased) activity, alternative combinations and order of ligation are investigated. The specific pair of 7-9 CTL epitope constructs yielding optimal activity are ligated and the resulting construct, encompassing all HTL epitopes, is evaluated for activity. Once again, up to two alternative orientations are investigated.

Based on these results an optimized plasmid configuration capable of effective delivery of a panel, e.g., of HIV epitopes, are selected for clinical trial evaluation. Of course, epitopes from any antigen of interest (infectious or disease-associated) can be used alone or in combination. This configuration will entail one or more HTL epitope constructs and one or more CTL epitope constructs. A combination of one long CTL and one long HTL epitope construct capable of effectively delivering all epitopes, is most preferable, as it simplifies further clinical development of the vaccine. In case undesirable interactions between the two constructs are observed when co-injected, injection of the different plasmids in the same animals, but in different injection sites, or at different points in time are examined. Alternatively, if a single CTL construct and HTL construct encoding all the desired epitopes is not identified, pools of constructs are considered for further development.

### Example 13

Evaluation and Characterization of CD8+ Lymphocyte Responses Induce Following Immunization With Multi-Epitope Vaccine

CD8+ lymphocyte responses were measured mostly relying on the ELISPOT technique. The ELISPOT assay is known in the art, and is regularly used in our laboratory. An automated Zeiss ELISPOT reader is also used as set forth herein. The assays utilized to measure CD8+ responses are primarily the IFNγ ELISPOT assay on freshly isolated cells as well as cells restimulated in vitro with peptide. In addition, in selected instances, chromium release assays are utilized. The results were correlated with the ones observed in the case of the ELISPOT assays. Tetramer staining on selected peptide/MHC combinations was also performed.

The clinical assay was developed and validated. The timing of this activity coincides with the period of time that follows selection of a clinical vaccine EpiGene™, and precedes the availability of actual samples from individuals enrolled in the clinical trial. Assays for CTL evaluation can be established based on experience in the art, for example, experience in establishing assays for CTL evaluations in the Phase I and II trials of the experimental HBV vaccine, Theradigm (Livingston et al, J Immunol, Vol. 159(3):1383-92 (1997); Heathcote et al., Hepatology, Vol. 30(2):531-6 (1999); Livingston et al., J Immunol, Vol. 162(5):3088-95 (1999)). Specifically, Ficoll-purified PBMC derived from normal subjects, as well from, e.g., unvaccinated volunteers can be used. As noted previously, other antigenic target(s) can be used in accordance with the invention.

### Example 14

Design of Optimized Multi-Epitope DNA-Based Vaccine Constructs

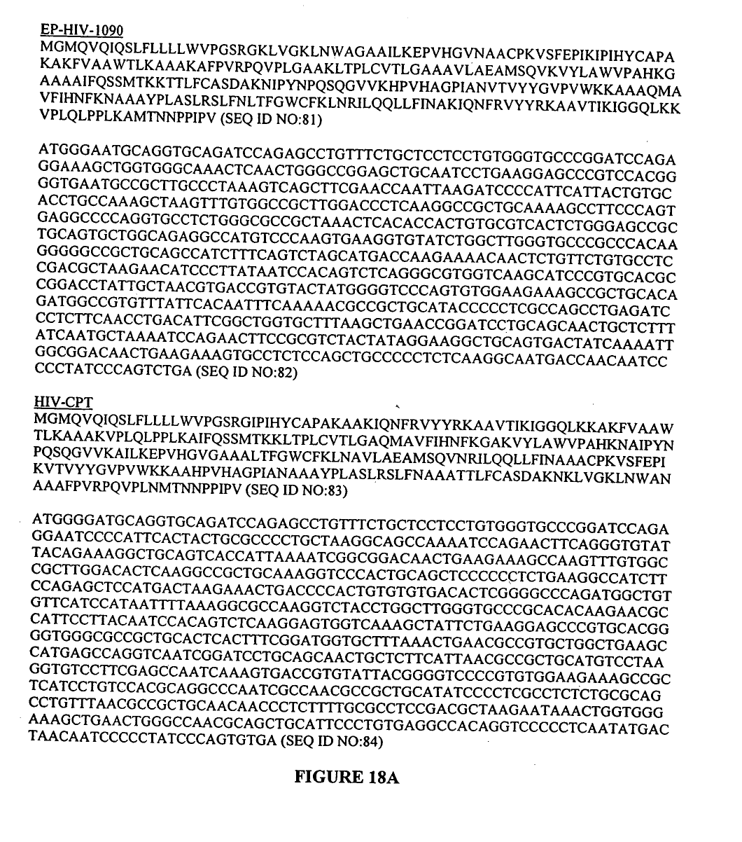

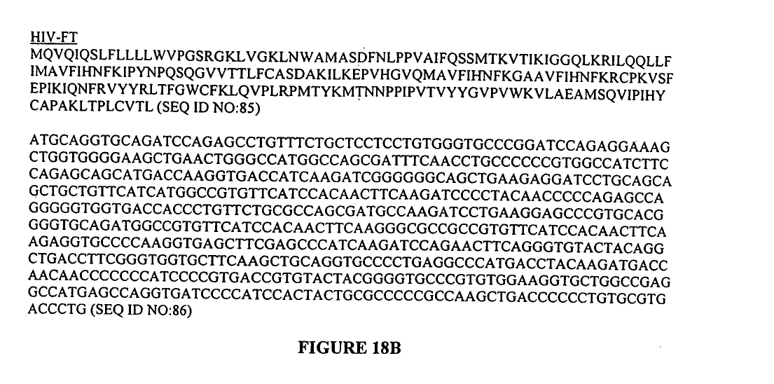

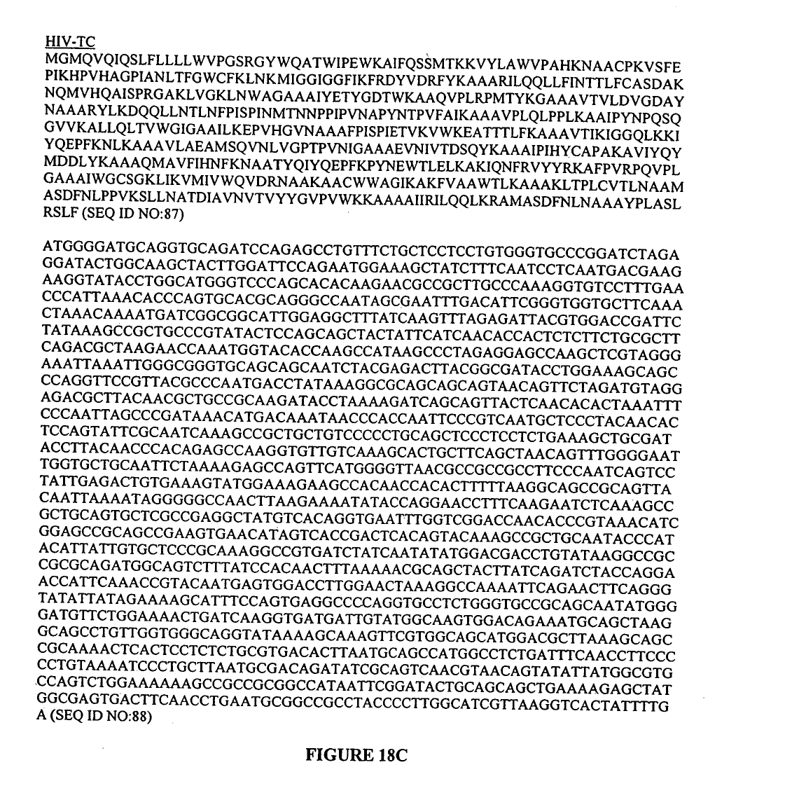

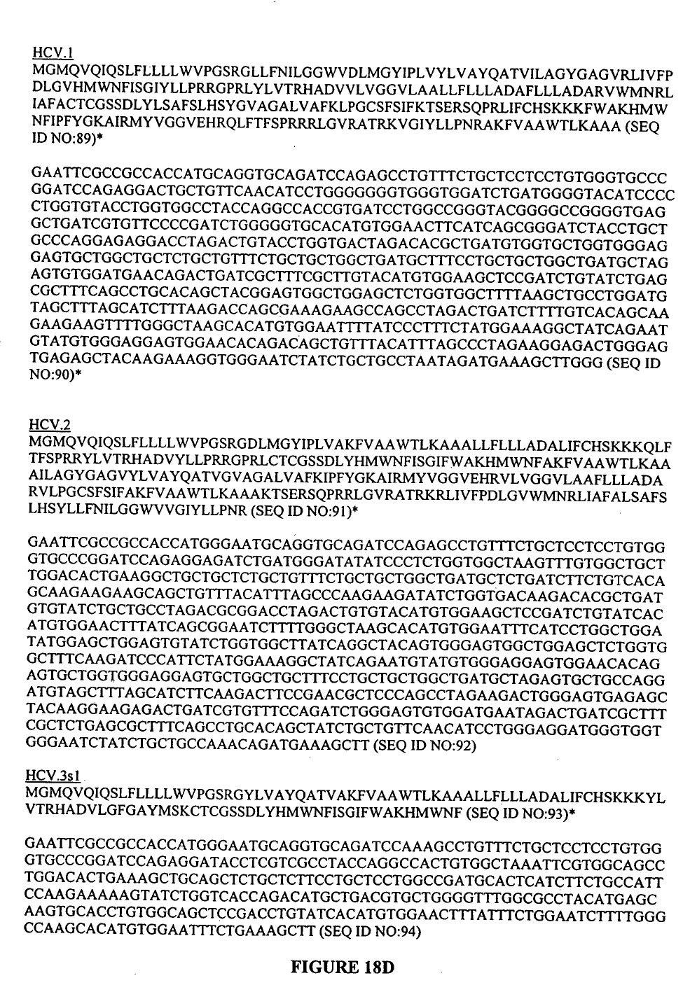

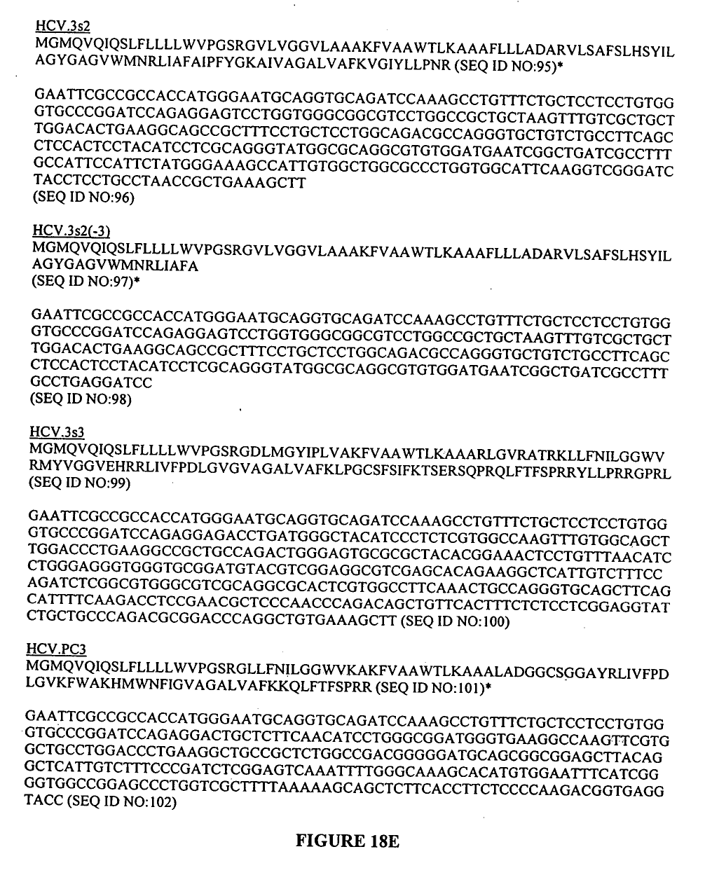

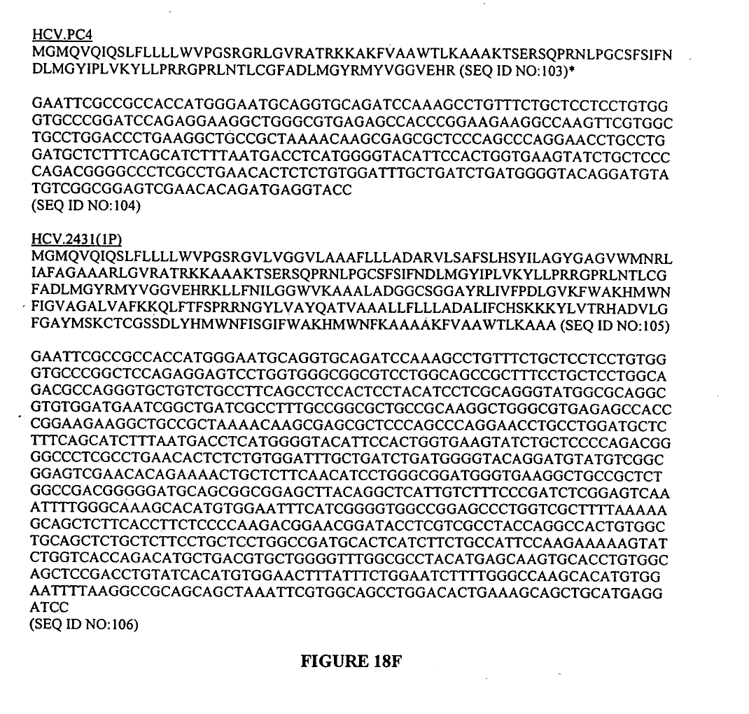

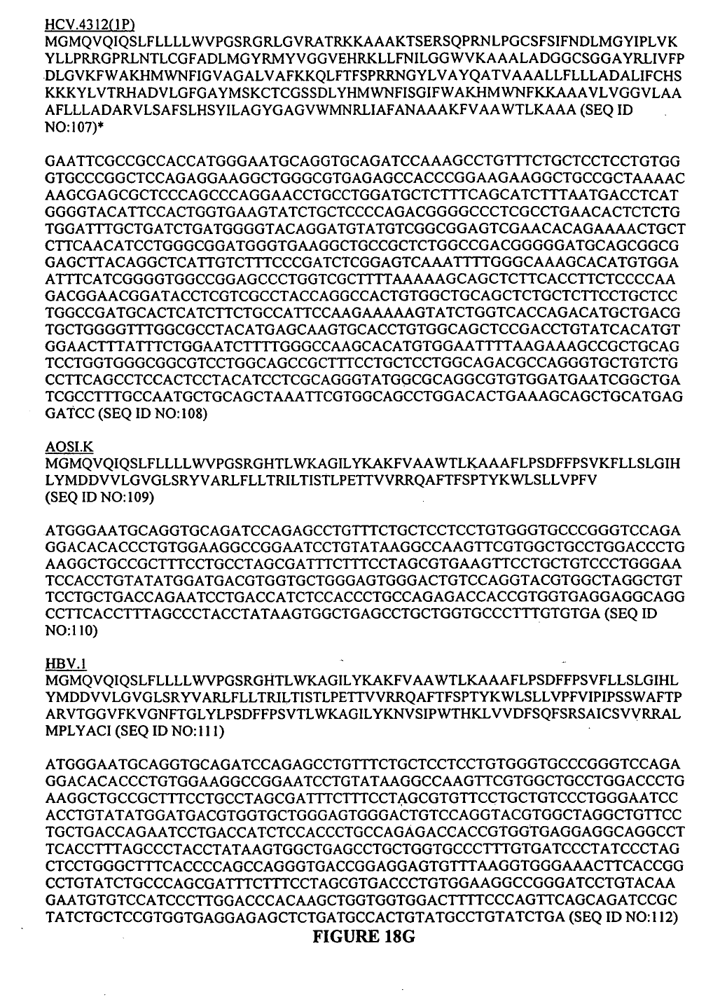

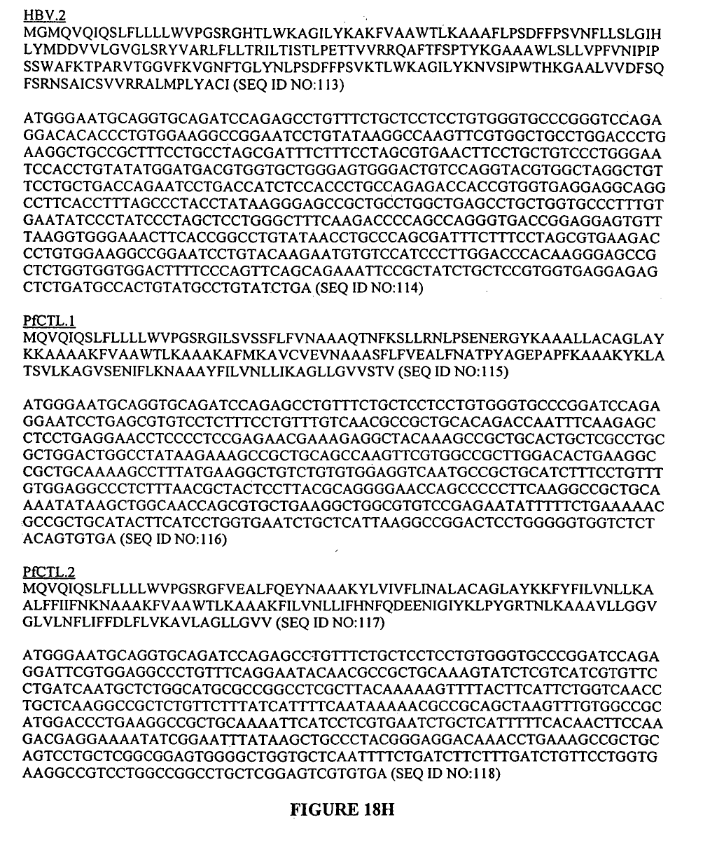

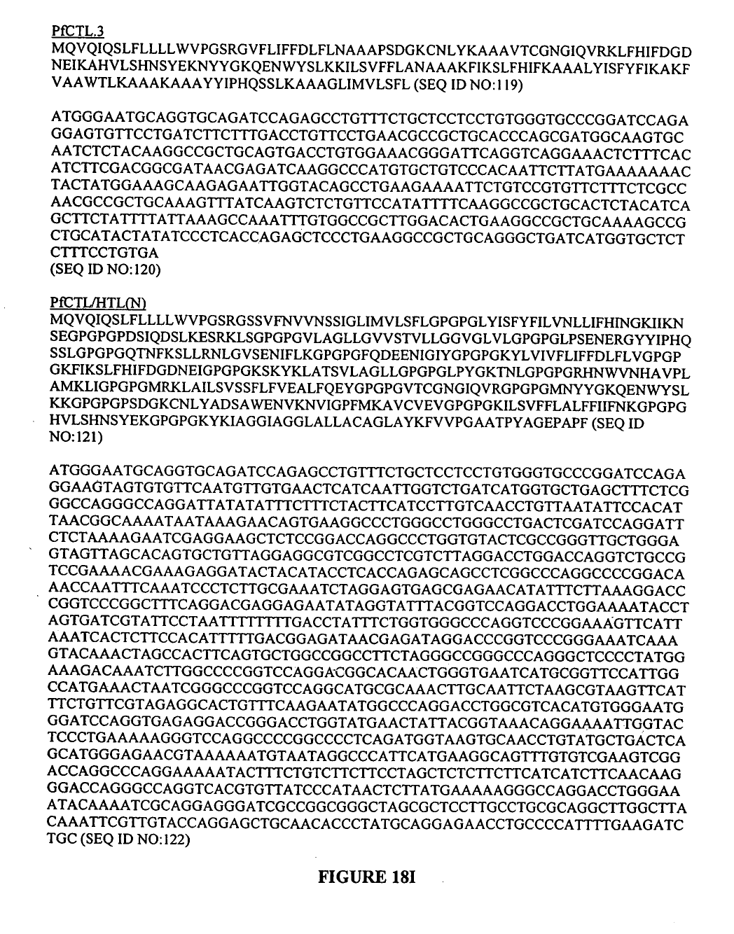

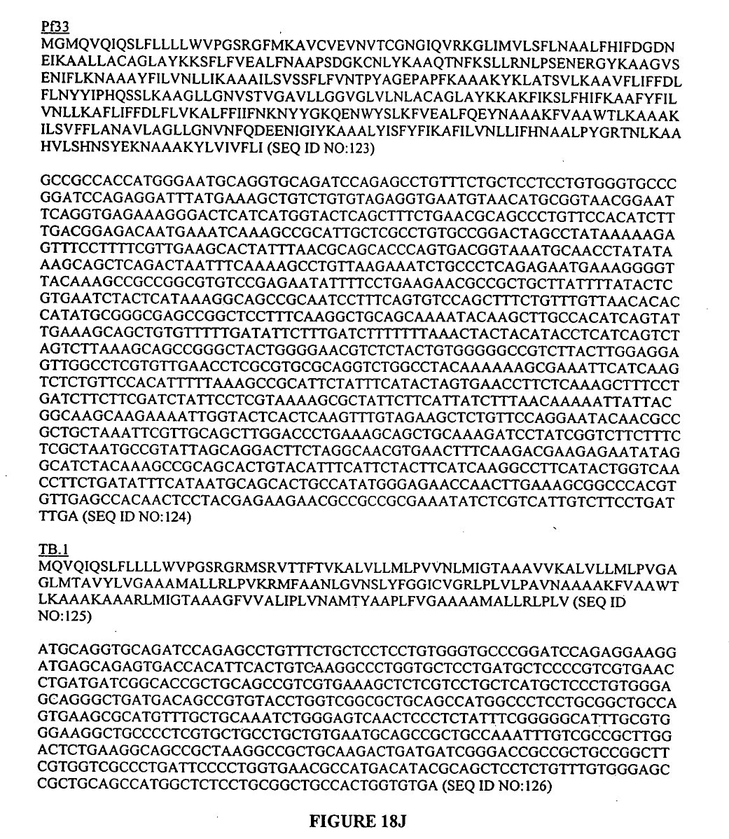

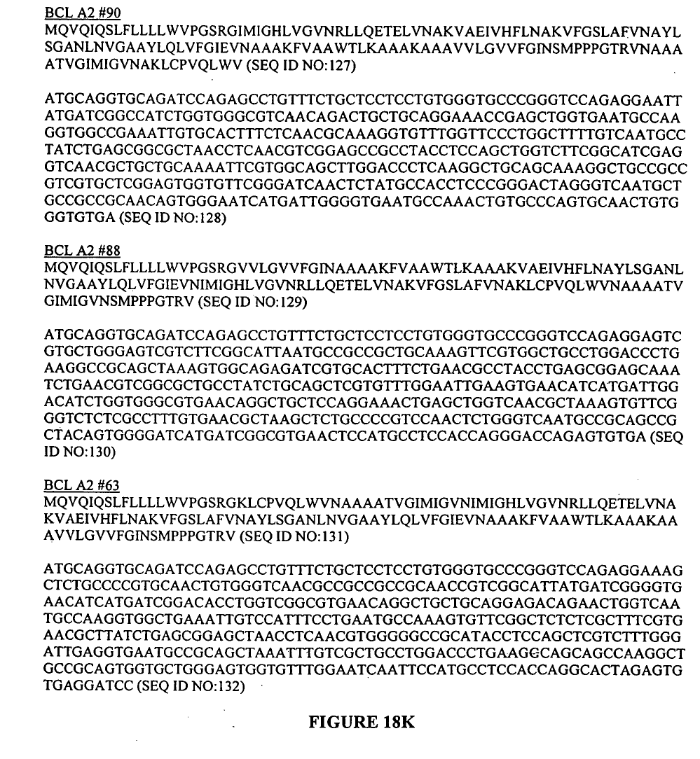

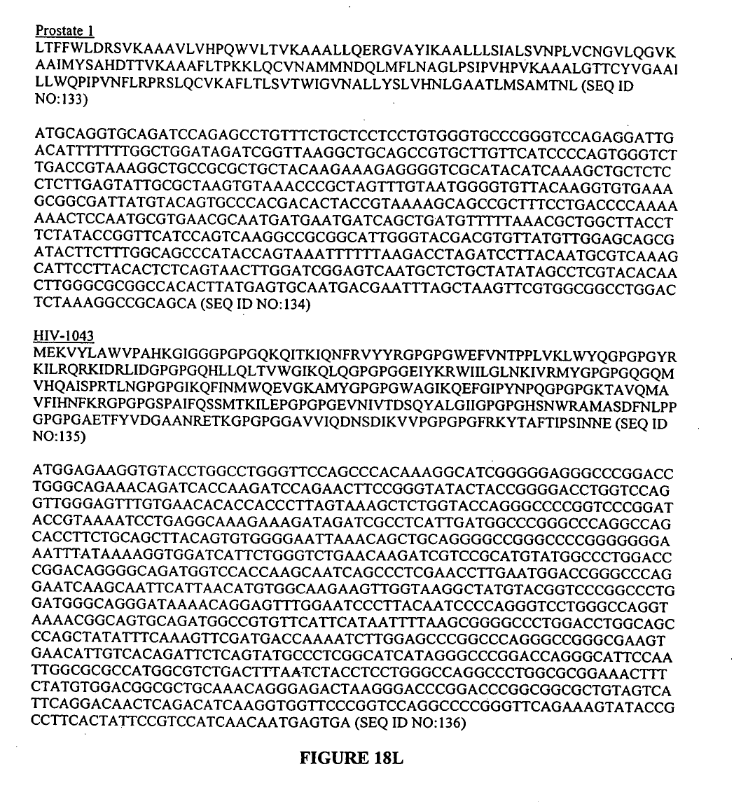

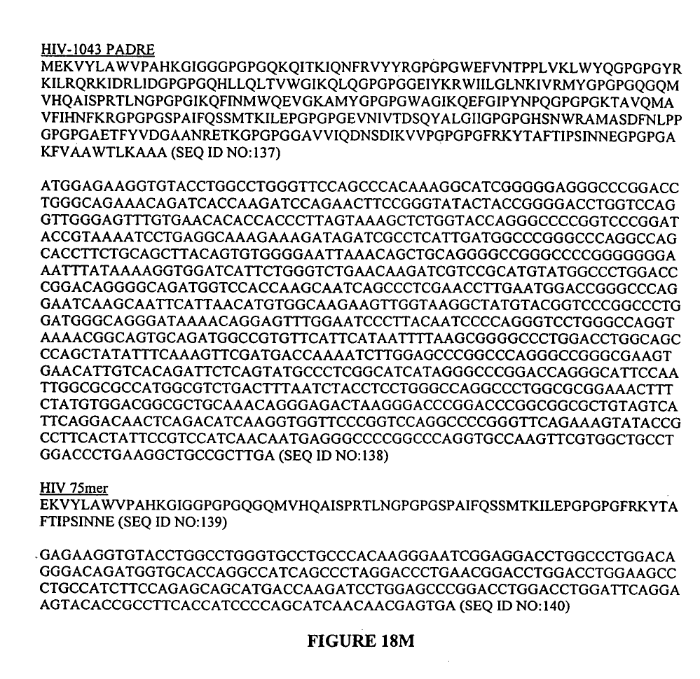

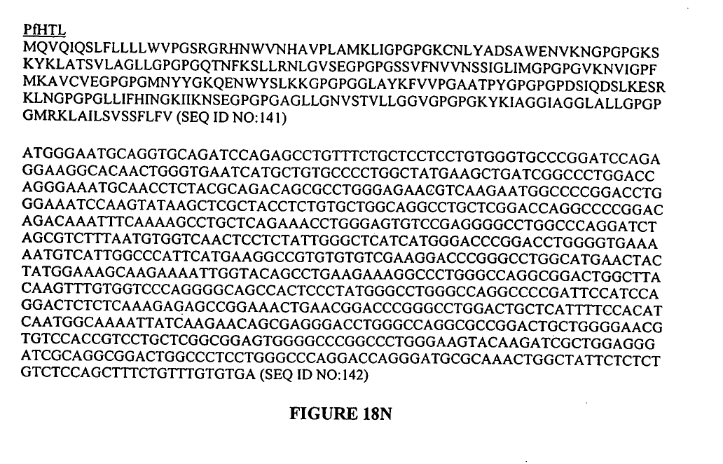

Optimized constructs were designed with the aid of the computer-assisted methods described above which simultaneously minimize the formation of junctional epitopes and optimize C+1 processing efficiency. The following motifs were utilized for junctional minimization: murine K^{b} (XXXX(FY)X₂₋₃(LIMV)); D^{b} (XXXXNX₂₋₃LMV)); human A2 (X(LM)X₆₋₇V); human A3/A11 (X(LIMV)X₆₋₇(KRY)); and human B7 (XPX₆₋₇(LIMVF)). The C+1 propensity values were calculated from the data presented in FIG. 6 and are as follows: K=2.2; N=2; G=1.8; T=1.5; A,F,S=1.33; W,Q=1.2; R=1.7; M,Y=1; I=0.86; L=0.76; V,D,H,E,P=0. Insertion of up to four amino acids was permitted. Examples of constructs designed by this procedure and other procedures set forth herein are depicted in FIG. 18. A number of these constructs were characterized in vitro and in vivo immunogenicity studies, which are set forth hereafter. FIG. 19 lists amino acid epitope sequences encoded by certain nucleic acid sequences in the multi-epitope constructs.

### Example 15

Immunogenicity Testing of Multi-Epitope CTL Constructs and Influence of Flanking Amino Acids

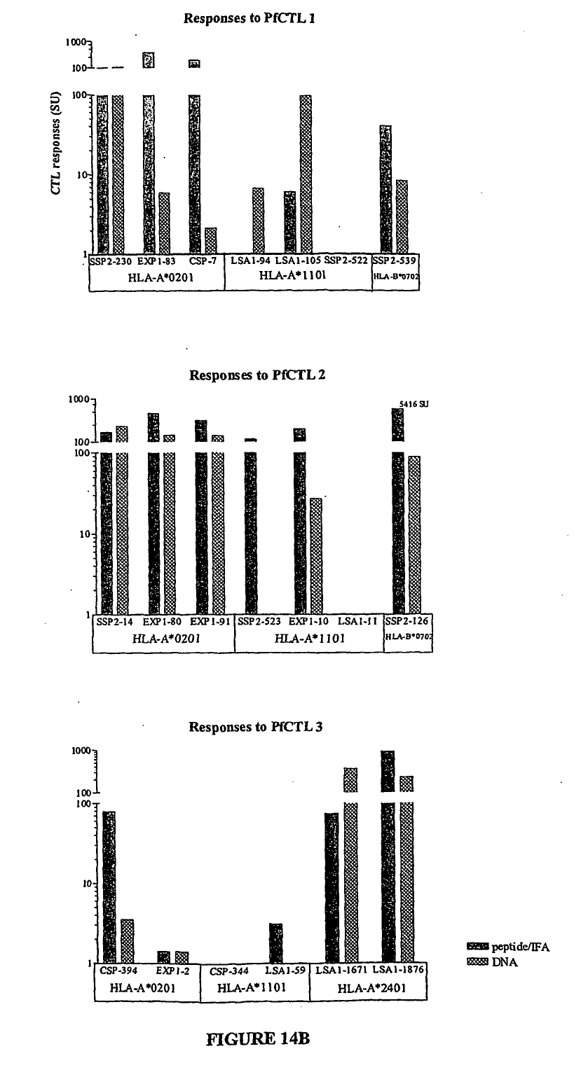

HLA transgenic mice were used for immunogenicity testing of different multi-epitope constructs. One group of mice were pretreated by injecting 50 μl of 10 μM cardiotoxin bilaterally into the tibialis anterior muscle, and then four or five days later, 100 μg of a DNA construct diluted in PBS was administered to the same muscle. In another group, each mouse was injected with a peptide emulsified in CFA, wherein the peptide corresponds to an epitope within the DNA construct administered to mice in the DNA injection group. Eleven to fourteen days after immunization, splenocytes from DNA vaccinated animals and peptide vaccinated animals were recovered and CTL activity was measured in one of several assays, including a standard ⁵¹Cr-release assay, an ELISPOT assay that measured γ-IFN production by purified CD8+ T-lymphocytes without peptide epitope-specific restimulation, and an in situ ELISA, which included an in vitro epitope-specific restimulation step with a peptide epitope. Examples of CTL activity induced by the EP-HIV-1090 construct upon stimulation with peptide epitopes are shown in FIG. 14A, and CTL activity induced by the PfCTL.1, PfCTL.2, and PFCTL.3 constructs upon stimulation with peptide epitopes are shown in FIG. 14B.

The effect of different amino acids in the C+1 flanking position was directly evaluated by inserting different amino acids at the C+1 position relative to the Core 18 epitope in the HBV.1 construct. The immunogenicity data clearly demonstrate reduced immunogenicity of the Core 18 epitope when it was flanked at the C+1 position by W, Y, or L (FIG. 6b). In contrast, insertion of a single K residue dramatically increased the CTL response to Core 18. Enhancement of CTL responses was also observed using R, C, N, or G at the C+1 position. These data clearly demonstrate that C+1 processing optimization can improve multi-epitope construct design.

### Example 16

Immunogenicity Testing of Multi-Epitope HTL Constructs and Influence of Spacer Sequences

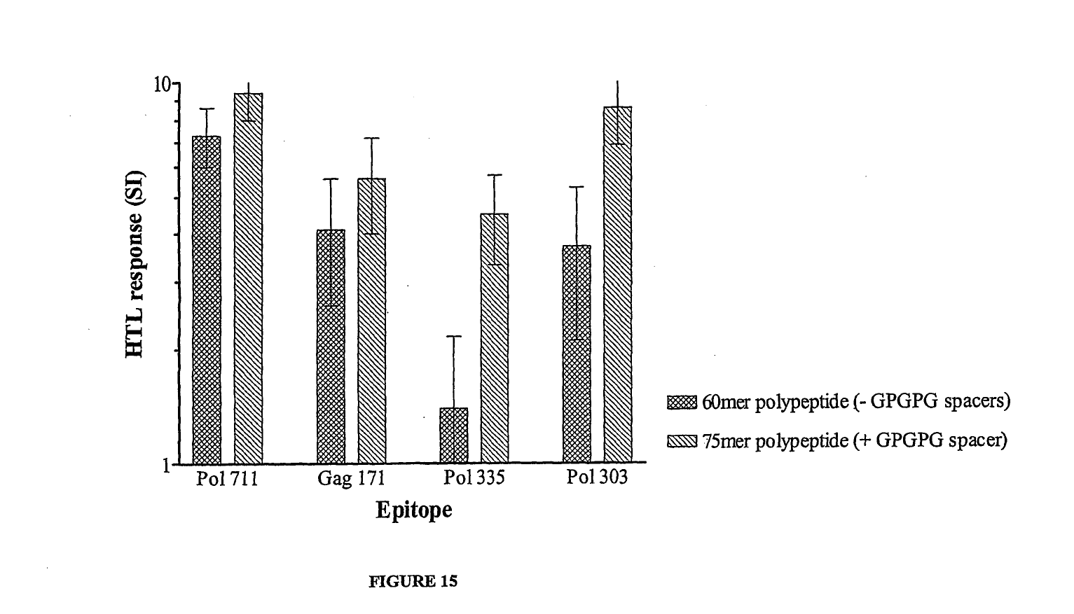

A universal spacer consisting of GPGPG was developed to separate HTL epitopes, thus disrupting junctional epitopes. The logic behind the design of this spacer is that neither G nor P are used as primary anchors, positions 1 and 6 in the core region of an HTL peptide epitope, by any known murine or human MHC Class IMHC Class II molecule. The gap of five amino acids introduced by this spacer separates adjacent epitopes so the amino acids of two epitopes cannot physically serve as anchors in the 1 and 6 positions. The utility of the GPGPG spacer was tested using synthetic peptides composed of four HIV-1 epitopes, one having three spacers and the other lacking spacers, known to bind mouse IA^{b}. HIV 75 mer was the construct having three GPGPG spacers and HIV 60 mer was the construct lacking the three spacers. Immunization of CB6F1 mice with the peptide in CFA induced HTL responses against 3 of 4 of the epitopes in the absence of the spacer but all epitopes were immunogenic when the spacer was present (FIG. 15). This evidence demonstrates that spacers can improve the performance of multi-epitope constructs.

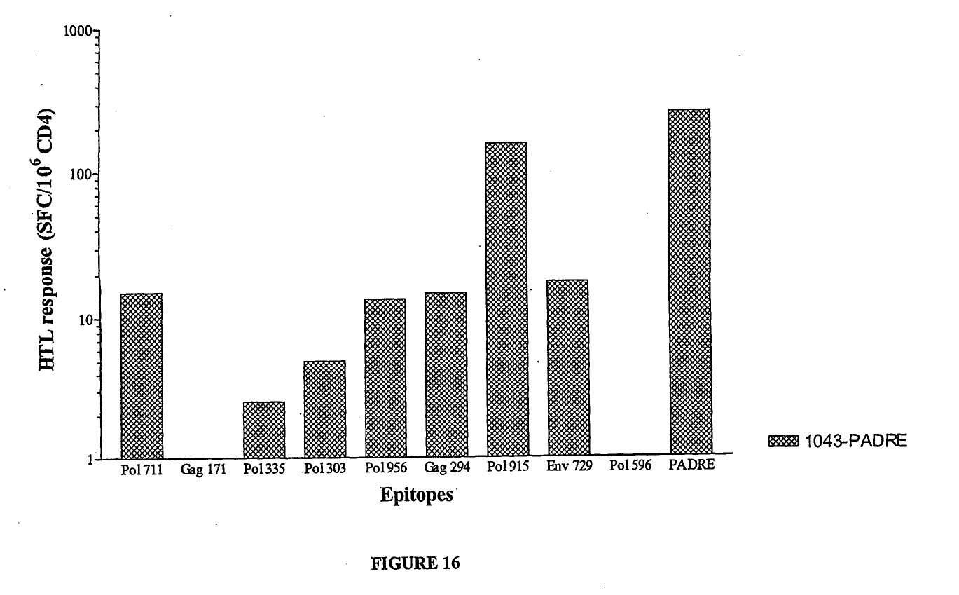

The ability of multi-epitope HTL DNA-based constructs to induce an HTL response in vivo was evaluated by intramuscular immunization of H2^{bxd} mice with an EP-HIV-1043-PADRE® construct. The EP-HIV-1043-PADRE® construct is set forth in FIG. 18, and the difference between EP-HIV-1043-PADRE® and EP-HIV-1043 is that the former includes a C-terminal GPGPG spacer followed by the PADRE® sequence AKFVAAWTLKAAA. Eleven days after immunization, no booster immunizations were administered, CD4 T cells were purified from the spleen, and peptide specific HTL responses were measured in a primary γ-IFN ELISPOT assay. Examples of HTL activity induced by constructs encoding HIV epitopes are shown in FIG. 16. Overall, the HTL responses induced by DNA immunization with the multi-epitope HIV HTL construct were generally of equal or greater magnitude than the responses induced by peptide immunization.

Thus, as described above, the invention provides a novel method and system for automatically analyzing polypeptide junctions, eliminating or reducing the number of junctional epitopes, and identifying spacer combinations to optimize the efficacy of multi-epitope constructs. Those skilled in the art will know, or be able to ascertain using no more than routine experimentation, many equivalents to the specific embodiments of the invention described herein. These equivalents are intended to be encompassed by the following claims.

1-38. (Cancelled).

39. A multi-epitope construct comprising a plurality of CTL epitope nucleic acids and a plurality of spacer nucleic acids, wherein:
the CTL epitope nucleic acids encode class I HLA epitope peptides of about eight to about eleven amino acids in length;
the spacer nucleic acids are positioned between the CTL epitope nucleic acids;
the spacer nucleic acids encode between one and eight amino acids;
one or more of the spacer nucleic acids encode an amino acid sequence that is different than the amino acid sequence encoded by other spacer nucleic acids; and
each of the spacer nucleic acids optimizes epitope processing and minimizes junctional epitopes.

40. The multi-epitope construct of claim 39, further comprising a targeting nucleic acid.

41. The multi-epitope construct of claim 39, further comprising a nucleic acid sequence encoding a HTL-specific epitope.

42. The multi-epitope construct of claim 39, wherein two or more of the spacer nucleic acids encodes an amino acid sequence that is different than the amino acid sequence encoded by other spacer nucleic acids.

43. The multi-epitope construct of claim 39, wherein one or more of the spacer nucleic acids encode an amino acid sequence comprising three consecutive alanine residues.

44. The multi-epitope construct of claim 39, wherein the epitope peptides are about nine amino acids in length.

45. The multi-epitope construct of claim 39, wherein the construct comprises five or more CTL epitope nucleic acids encoding class I HLA epitope peptides selected from the group consisting of FLPSDFFPSV (SEQ ID NO:284); FLLTRILTI (SEQ ID NO:285); WLSLLVPFV (SEQ ID NO:286); GLSRYVARL (SEQ ID NO:287); YMDDVVLGV (SEQ ID NO:288); FLLSLGIHL (SEQ ID NO:290); ALMPLYACI (SEQ ID NO:291); STLPETTVVRR (SEQ ID NO:293); HTLWKAGILYK (SEQ ID NO:294); TLWKAGILYK (SEQ ID NO:295); LVVDFSQFSR (SEQ ID NO:296); NVSIPWTHK (SEQ ID NO:297); SAICSVVRR (SEQ ID NO:298); KVGNFTGLY (SEQ ID NO:299); QAFTFSPTYK (SEQ ID NO:300); LPSDFFPSV (SEQ ID NO:301); IPIPSSWAF (SEQ ID NO:302); TPARVTGGVF (SEQ ID NO:303); HPAAMPHLL (SEQ ID NO:369); WMMWYWGPSLY (SEQ ID NO:370); DLLDTASALY (SEQ ID NO:371); LTFGRETVLEY (SEQ ID NO:372); LSLDVSAAFY (SEQ ID NO:373); SWPKFAVPNL (SEQ ID NO:374); RFSWLSLLVPF (SEQ ID NO:375); EYLVSFGVW (SEQ ID NO:376); KYTSFPWLL (SEQ ID NO:377).

46. The multi-epitope construct of claim 39, wherein the construct comprises five or more CTL epitope nucleic acids encoding class I HLA epitope peptides selected from the group consisting of FLPSDFFPSV (SEQ ID NO:284); FLLTRILTI (SEQ ID NO:285); WLSLLVPFV (SEQ ID NO:286); FLLSLGIHL (SEQ ID NO:290); YMDDVVLGV (SEQ ID NO:288); STLPETTVVRR (SEQ ID NO:293); HTLWKAGILYK (SEQ ID NO:294); SAICSVVRR (SEQ ID NO:298); QAFTFSPTYK (SEQ ID NO:300); IPIPSSWAF (SEQ ID NO:302); TPARVTGGVF (SEQ ID NO:303); HPAAMPHLL (SEQ ID NO:369); FPHCLAFSYM (SEQ ID NO:378); WMMWYWGPSLY (SEQ ID NO:370); DLLDTASALY (SEQ ID NO:371); LTFGRETVLEY (SEQ ID NO:372); LSLDVSAAFY (SEQ ID NO:373); SWPKFAVPNL (SEQ ID NO:374); RFSWLSLLVPF (SEQ ID NO:375); EYLVSFGVW (SEQ ID NO:376); KYTSFPWLL (SEQ ID NO:377).

47. The multi-epitope construct of claim 39, wherein the construct comprises five or more CTL epitope nucleic acids encoding class I HLA epitope peptides selected from the group consisting of FLPSDFFPSV (SEQ ID NO:284); FLLTRILTI (SEQ ID NO:285); WLSLLVPFV (SEQ ID NO:286); GLSRYVARL (SEQ ID NO:287); YMDDVVLGV (SEQ ID NO:288); FLLSLGIHL (SEQ ID NO:290); STLPETTVVRR (SEQ ID NO:293); HTLWKAGILYK (SEQ ID NO:294); LVVDFSQFSR (SEQ ID NO:296); NVSIPWTHK (SEQ ID NO:297); SAICSVVRR (SEQ ID NO:298); QAFTFSPTYK (SEQ ID NO:300); LPSDFFPSV (SEQ ID NO:301); IPIPSSWAF (SEQ ID NO:302); TPARVTGGVF (SEQ ID NO:303); HPAAMPHLL (SEQ ID NO:369); FPHCLAFSYM (SEQ ID NO:378); YPALMPLYACI (SEQ ID NO:379); WMMWYWGPSLY (SEQ ID NO:370); DLLDTASALY (SEQ ID NO:371); LTFGRETVLEY (SEQ ID NO:372); ASFCGSPY (SEQ ID NO:380); LSLDVSAAFY (SEQ ID NO:373); ILLLCLIFLL (SEQ ID NO:381); RWMCLRRFII (SEQ ID NO:382); SWPKFAVPNL (SEQ ID NO:374); RFSWLSLLVPF (SEQ ID NO:375); LWFHISCLTF (SEQ ID NO:383); EYLVSFGVW (SEQ ID NO:376) KYTSFPWLL (SEQ ID NO:377).

48. The multi-epitope construct of claim 39, wherein the construct comprises five or more CTL epitope nucleic acids encoding class I HLA epitope peptides selected from the group consisting of VLAEAMSQV (SEQ ID NO:143); MTNNPPIPV (SEQ ID NO:144); MASDFNLPPV (SEQ ID NO:145); KLVGKLNWA (SEQ ID NO:146); LVGPTPVNI (SEQ ID NO:147); ILKEPVHGV (SEQ ID NO:148); KAACWWAGI (SEQ ID NO:149); KMIGGIGGFI (SEQ ID NO:150); RAMASDFNL (SEQ ID NO:151); TLNFPISPI (SEQ ID NO:152); KLTPLCVTL (SEQ ID NO:153); LLQLTVWGI (SEQ ID NO:154); SLLNATDIAV (SEQ ID NO:155); LTFGWCFKL (SEQ ID NO:156); AIIILQQL (SEQ ID NO:157); RILQQLLFI (SEQ ID NO:158); QMAVFIHNFK (SEQ ID NO:159); KVYLAWVPAHK (SEQ ID NO:160); KIQNFRVYYR (SEQ ID NO:161); AIFQSSMTK (SEQ ID NO:162); VTIKIGGQLK (SEQ ID NO:163); TTLFCASDAK (SEQ ID NO:164); VTVYYGVPVWK (SEQ ID NO:165); QVPLRPMTYK (SEQ ID NO:166); VMIVWQVDR (SEQ ID NO:167); QMVHQAISPR (SEQ ID NO:168); YPLASLRSLF (SEQ ID NO:169); HPVHAGPIA (SEQ ID NO:170); FPISPIETV (SEQ ID NO:171); IPYNPQSQGVV (SEQ ID NO:1 72); IPIHYCAPA (SEQ ID NO:1 73); CPKVSFEPI (SEQ ID NO:174); FPVRPQVPL (SEQ ID NO:175); VPLQLPPL (SEQ ID NO:176); EVNIVTDSQY (SEQ ID NO:177); FRDYVDRFY (SEQ ID NO:178); VIYQYMDDLY (SEQ ID NO:179); VTVLDVGDAY (SEQ ID NO:180); IYQEPFKNL (SEQ ID NO:181); PYNTPVFAI (SEQ ID NO:182); TYQIYQEPF (SEQ ID NO:183); YWQATWIPEW (SEQ ID NO:184); IWGCSGKLI (SEQ ID NO:185); RYLKDQQLL (SEQ ID NO:186); VWKEATTTLF (SEQ ID NO:187); IYETYGDTW (SEQ ID NO:188); PYNEWTLEL (SEQ ID NO:189); LLFNILGGWV (SEQ ID NO:208); FLLLADARV (SEQ ID NO:209); YLVAYQATV (SEQ ID NO:210); RLIVFPDLGV (SEQ ID NO:21 1); DLMGYIPLV (SEQ ID NO:212); WMNRLIAFA (SEQ ID NO:213); VLVGGVLAA (SEQ ID NO:214); HMWNFISGI (SEQ ID NO:215); ILAGYGAGV (SEQ ID NO:216); YLLPRRGPRL (SEQ ID NO:217); LLFLLLADA (SEQ ID NO:218); YLVTRHADV (SEQ ID NO:219); KTSERSQPR (SEQ ID NO:220); RLGVRATRK (SEQ ID NO:221); QLFTFSPRR (SEQ ID NO:222); RMYVGGVEHR (SEQ ID NO:223); LIFCHSKKK (SEQ ID NO:224); GVAGALVAFK (SEQ ID NO:225); VAGALVAFK (SEQ ID NO:226); LGFGAYMSK (SEQ ID NO:227); LPGCSFSIF (SEQ ID NO:228); LSAFSLHSY (SEQ ID NO:229); CTCGSSDLY (SEQ ID NO:230); LTDPSHITA (SEQ ID NO:231); LTCGFADLMGY (SEQ ID NO:232); LADGGCSGGAY (SEQ ID NO:233); FWAKHMWNF (SEQ ID NO:234); RMILMTHFF (SEQ ID NO:235); VMGSSYGF (SEQ ID NO:236); FWAKHMWNFI (SEQ ID NO:237); FMKAVCVEV (SEQ ID NO:238); GLLGVVSTV (SEQ ID NO:239); ILSVSSFLFV (SEQ ID NO:240); QTNFKSLLR (SEQ ID NO:241); GVSENIFLK (SEQ ID NO:242); LLACAGLAYK (SEQ ID NO:243); TPYAGEPAPF (SEQ ID NO:244); LPSENERGY (SEQ ID NO:245); KYKLATSVL (SEQ ID NO:246); SFLFVEALF (SEQ ID NO:247); YFILVNLLI (SEQ ID NO:248); FLIFFDLFLV (SEQ ID NO:249); VLAGLLGVV (SEQ ID NO:250); VLLGGVGLVL (SEQ ID NO:251); LACAGLAYK (SEQ ID NO:252); ALFFIIFNK (SEQ ID NO:253); FILVNLLIFH (SEQ ID NO:254); LPYGRTNL (SEQ ID NO:255); FVEALFQEY (SEQ ID NO:256); FQDEENIGIY (SEQ ID NO:257); FYFILVNLL (SEQ ID NO:258); KYLVIVFLI (SEQ ID NO:259); GLIMVLSFL (SEQ ID NO:260); KILSVFFLA (SEQ ID NO:261); VTCGNGIQVR (SEQ ID NO:262); HVLSHNSYEK (SEQ ID NO:263); PSDGKCNLY (SEQ ID NO:264); YYIPHQSSL (SEQ ID NO:265); KFIKSLFHIF (SEQ ID NO:266); VFLIFFDLFL (SEQ ID NO:267); LFHIFDGDNEI (SEQ ID NO:268); YYGKQENWYSL (SEQ ID NO:269); LYISFYFI (SEQ ID NO:270); FLPSDFFPSV (SEQ ID NO:284); FLLTRILTI (SEQ ID NO:285); WLSLLVPFV (SEQ ID NO:286); GLSRYVARL (SEQ ID NO:287); YMDDVVLGV (SEQ ID NO:288); ILRGTSFVYV (SEQ ID NO:289); FLLSLGIHL (SEQ ID NO:290); ALMPLYACI (SEQ ID NO:291); GLSPTVWLSV (SEQ ID NO:292); STLPETTVVRR (SEQ ID NO:293); HTLWKAGILYK (SEQ ID NO:294); TLWKAGILYK (SEQ ID NO:295); LVVDFSQFSR (SEQ ID NO:296); NVSIPWTHK (SEQ ID NO:297); SAICSVVRR (SEQ ID NO:298); KVGNFTGLY (SEQ ID NO:299); QAFTFSPTYK (SEQ ID NO:300); LPSDFFPSV (SEQ ID NO:301); IPIPSSWAF (SEQ ID NO:302); TPARVTGGVF (SEQ ID NO:303); RMSRVTTFTV (SEQ ID NO:304); ALVLLMLPVV (SEQ ID NO:305); LMIGTAAAVV (SEQ ID NO:306); ALVLLMLPV (SEQ ID NO:307); GLMTAVYLV (SEQ ID NO:308); MALLRLPV (SEQ ID NO:309); RMFAANLGV (SEQ ID NO:310); SLYFGGICV (SEQ ID NO:311); RLPLVLPAV (SEQ ID NO:312); RLMIGTAAA (SEQ ID NO:313); FVVALIPLV (SEQ ID NO:314); MTYAAPLFV (SEQ ID NO:315); AMALLRLPLV (SEQ ID NO:316); KLCPVQLWV (SEQ ID NO:317); ATVGIMIGV (SEQ ID NO:318); IMIGHLVGV (SEQ ID NO:319); RLLQETELV (SEQ ID NO:320); KVAEIVHFL (SEQ ID NO:321); VVLGVVFGI (SEQ ID NO:325); SMPPPGTRV (SEQ ID NO:326); LTFFWLDRSV (SEQ ID NO:327); TLMSAMTNL (SEQ ID NO:328); IMYSAHDTTV (SEQ ID NO:329); GLPSIPVHPV (SEQ ID NO:330); LLQERGVAYI (SEQ ID NO:331); LLYSLVHNL (SEQ ID NO:332); MMNDQLMFL (SEQ ID NO:333); FLTLSVTWIGV (SEQ ID NO:334); ALGTTCYV (SEQ ID NO:335); FLTPKKLQCV (SEQ ID NO:336); LLLSIALSV (SEQ ID NO:337); VLVHPQWVLTV (SEQ ID NO:338); FLRPRSLQCV (SEQ ID NO:339); PLVCNGVLQGV (SEQ ID NO:340).

49. The multi-epitope construct of claim 39 having the nucleotide sequence selected from the group consisting of HBV-2A, HBV-2B, HBV-21A, HBV-21B, HBV-30B, HBV-30C, HBV-30CL and a nucleotide sequence that hybridizes to any of the foregoing.

50-55. (Cancelled).

56. A multi-epitope construct comprising a plurality of HTL epitope nucleic acids and a plurality of spacer nucleic acids, wherein:
the HTL epitope nucleic acids encode class II HLA epitopes of about seven to about seventeen amino acids in length;
the spacer nucleic acids are positioned between the HTL epitope nucleic acids;
the spacer nucleic acids encode five or more amino acids, wherein each of the spacer nucleic acids optimizes epitope processing and minimizes junctional epitopes: and
the construct comprises five or more HTL epitope nucleic acids encoding class II HLA epitope peptides selected from the group consisting of MGTSFVYVPSALNPAD (SEQ ID NO:400); LCQVFADATPTGWGL (SEQ ID NO:401); RHYLHTLWKAGILYK (SEQ ID NO:402); PHHTALRQAILCWGELMTLA (SEQ ID NO:403); ESRLVVDFSQFSRGN (SEQ ID NO:404); PFLLAQFTSAICSVV (SEQ ID NO:405); LVPFVQWFVGLSPTV (SEQ ID NO:406); LHLYSHPIILGFRKI (SEQ ID NO:407); SSNLSWLSLDVSAAF (SEQ ID NO:408); LQSLTNLLSSNLSWL (SEQ ID NO:409); AGFFLLTRILTIPQS (SEQ ID NO:410); VSFGVWIRTPPAYRPPNAPI (SEQ ID NO:411); VGPLTVNEKRRLKLI (SEQ ID NO:412); KQCFRKLPVNRPIDW (SEQ ID NO:413); AANWILRGTSFVYVP (SEQ ID NO:414); KQAFTFSPTYKAFLC (SEQ ID NO:415); AKFVAAWTLKAAA (SEQ ID NO:69).

57. A multi-epitope construct comprising a plurality of HTL epitope nucleic acids and a plurality of spacer nucleic acids, wherein:
the HTL epitope nucleic acids encode class II HLA epitopes of about seven to about seventeen amino acids in length;
the spacer nucleic acids are positioned between the HTL epitope nucleic acids;
the spacer nucleic acids encode five or more amino acids, wherein each of the spacer nucleic acids optimizes epitope processing and minimizes junctional epitopes; and
the construct comprises five or more HTL epitope nucleic acids encoding class II HLA epitope peptides selected from the group consisting of KRWIILGLNKIVRMY (SEQ ID NO:190); WEFVNTPPLVKLWYQ (SEQ ID NO:191); QKQITKIQNFRVYYR (SEQ ID NO:192); KVYLAWVPAHKGIGG (SEQ ID NO:193); GEIYKRWIILGLNKI (SEQ ID NO:194); EKVYLAWVPAHKGIG (SEQ ID NO:195); QHLLQLTVWGIKQLQ (SEQ ID NO:196); QGQMVHQAISPRTLN (SEQ ID NO:197); SPAIFQSSMTKILEP (SEQ ID NO:198); IKQFINMWQEVGKAMY (SEQ ID NO:199); FRKYTAFTIPSINNE (SEQ ID NO:200); HSNWRAMASDFNLPP (SEQ ID NO:201); KTAVQMAVFIHNFKR (SEQ ID NO:202); YRKILRQRKIDRLID (SEQ ID NO:203); WAGIKQEFGIPYNPQ (SEQ ID NO:204); EVNIVTDSQYALGII (SEQ ID NO:205); AETFYVDGAANRETK (SEQ ID NO:206); GAVVIQDNSDIKVVP (SEQ ID NO:207); MRKLAILSVSSFLFV (SEQ ID NO:271); MNYYGKQENWYSLKK (SEQ ID NO:272); SSVFNVVNSSIGLIM (SEQ ID NO:273); RHNWVNHAVPLAMKLI (SEQ ID NO:274); PDSIQDSLKESRKLN (SEQ ID NO:275); KCNLYADSAWENVKN (SEQ ID NO:276); VKNVIGPFMKAVCVE (SEQ ID NO:277); KYKIAGGIAGGLALL (SEQ ID NO:278); GLAYKFVVPGAATPY (SEQ ID NO:279); KSKYKLATSVLAGLL (SEQ ID NO:280); AGLLGNVSTVLLGGV (SEQ ID NO:281); LLIFHINGKIIKNSE (SEQ ID NO:282); QTNFKSLLRNLGVSE (SEQ ID NO:283).
